(19) United States
(12) Patent Application Publication
(10) Pub. No.: US 2022/0088138 A1
(43) Pub. Date: Mar. 24, 2022

(54) METHODS FOR DETECTING AND MODULATING THE EMBRYONIC-FETAL TRANSITION IN MAMMALIAN SPECIES

(71) Applicant: AgeX Therapeutics, Inc., Alameda, CA (US)

(72) Inventors: Michael D. West, Mill Valley, CA (US); Dana Larocca, Alameda, CA (US)

(21) Appl. No.: 17/543,020

(22) Filed: Dec. 6, 2021

Related U.S. Application Data
(63) Continuation of application No. 16/211,690, filed on Dec. 6, 2018.
Application No. 16/211,690 is a continuation of application No. PCT/US2017/036452, filed on Jun. 7, 2017.
(60) Provisional application No. 62/442,219, filed on Jan. 4, 2017.
(60) Provisional application No. 62/347,075, filed on Jun. 7, 2016.

(51) Int. Cl. A61K 38/18; A61K 38/22; A61K 9/06; A61K 31/19; A61K 31/7105; A61K 31/715; A61K 31/713; A61K 45/06; A61K 9/00; A61K 47/36

(52) U.S. Cl. CPC A61K 38/18; A61K 38/22; A61K 9/06; A61K 31/19; A61K 47/36; A61K 31/715; A61K 31/713; A61K 45/06; A61K 9/0024; A61K 31/7105

(57) ABSTRACT

Aspects of the present invention include algorithms, methods and compositions related to the modulation of molecules regulating the mammalian transition from embryonic to fetal development. Methods and compositions for the use of such modulations to increase the regenerative potential in fetal and adult tissues otherwise incapable of scarless regeneration are also presented.

### CROSS-REFERENCE TO RELATED APPLICATIONS

This application is a continuation of International Patent Application No. PCT/US2017/036452, filed Jun. 7, 2017, which claims priority to U.S. provisional patent application No. 62/347,075 filed on Jun, 7, 2016, and U.S. provisional patent application No. 62/442,219, filed Jan. 4, 2017.

The entire contents of the aforementioned applications are expressly incorporated herein by reference in their entirety.

### BACKGROUND

Advances in stem cell technology, such as the isolation and propagation in vitro of human pluripotent stem (hPS) cells constitute an important new area of medical research. hPS cells have a demonstrated potential to be propagated in the undifferentiated state and then to be induced subsequently to differentiate into any and all of the cell types in the human body, including complex tissues. This has led, for example, to the prediction that many diseases resulting from the dysfunction of cells may be amenable to treatment by the administration of human embryonic stem cell-derived of various differentiated types (Thomson et al., Science 282:1145-1147 (1998)). With regard to differentiating hPS cells into desired cell types, the potential to clonally isolate lines of human embryonic progenitor cells provides a means to propagate novel highly purified cell lineages with a prenatal pattern of gene expression useful for regenerating tissues such as skin in a scarless manner Such cell types have important applications in research, and for the manufacture of cell-based therapies (see PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each incorporated herein by reference).

More recently, the potential of pluripotent stem cells and derived embryoid bodies for in vitro self-assembly into 3-dimensional organoids has generated interest as a potential pathway for both obtaining tissue for transplantation (Singh et al, Stem Cells Dev. 2015. 24(23): 2778-95) as well as modeling human embryonic development. In contrast to embryonic cells, fetal and adult-derived cells often show reduced potential for organogenesis in vitro and epimorphic regeneration in vivo. Epimorphic regeneration, sometimes referred to as “epimorphosis,” refers to a type of tissue regeneration wherein a blastema of relatively undifferentiated mesenchyme proliferates at the site of injury and then the cells differentiate to restore the original tissue histology. The developmental timing of the loss of epimorphic potential cannot be fixed precisely, and likely varies with tissue type, nevertheless, the embryonic-fetal transition (EFT), or eight weeks of human development (Carnegie Stage 23; O'Rahilly, R., F. Müller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). Correlations between species show increased regenerative potential in the embryonic or larval state (reviewed in Morgan, T. H. (1901). Regeneration (New York: The MacMillan Company); also Sanchez Alvarado, A., and Tsonis, P. A. (2006). Bridging the regeneration gap: genetic insights from diverse animal models (Nat. Rev. Genet. 7, 873-884) suggest that tissue regeneration, as opposed to scarring, reflects the presence of an embryonic as opposed to fetal or adult phenotype. In the case of some species, a change in developmental timing (heterochrony) correlates with profound regenerative potential such as is the case in the developmental arrest in larval development (heterochrony) and limb regeneration observed in the Mexican salamander axolotl (A. mexicanum) (Voss, S. R. et al, Thyroid hormone responsive QTL and the evolution of paedomorphic salamanders. Heredity (2012) 109, 293-298. In contrast, some animals such as the African Spiny mouse (Acomys cahirinus) show a profound potential for skin regeneration in the absence of overt heterochrony, perhaps reflecting uncharacterized molecular alterations (Gawriluk, T. R., 2016. Comparative analysis of ear-hole closure identifies epimorphic regeneration as a discrete trait in mammals Nature Commun. 7:11164).

Despite these observations, there are limited markers of the EFT to test the role of specific molecules in epimorphic regeneration. We previously disclosed compositions and methods related to markers of the EFT in mammalian species and their use in modulating tissue regeneration (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, the disclosures of which are hereby incorporated by reference in their entirety. Nevertheless, additional molecular regulators and methods for modulating the EFT are needed for research and therapy in regenerative medicine and cancer.

Early candidates for regulators of heterochrony were identified in C. elegans. These included lin-28/let-7 (Ambros, V. and Horvitz, H. R. (1984). Heterochronic mutants of the nematode Caenorhabditis elegans. Science 226, 409-416). More recently, transgenic expression of the paralog Lin28a in mice has been reported to increase skin regeneration and amputated digit regrowth following wounding and to increase markers of oxidative phosphorylation (Shyh-Chang, N. et al 2013. Lin28 Enhances Tissue Repair by Reprogramming Cellular Metabolism. Cell 155, 778-792). However, the regenerative potential in said mice is not comparable to the profound epimorphosis observed in Acomys cahirinus. The lin-28/let-7 axis has also been observed to be activated in a number of cancer cell types (Jiang, S. and Baltimore, D. 2016. RNA-binding protein Lin28 in cancer and immunity, Cancer Lett. 375(1):108-13). The abnormal expression of LIN28 in cancer suggests that perhaps the reason for the natural selection for repression of regenerative potential at the EFT is that in most vertebrates selection for this trait, while potentially limiting survival following injury, could function as a tumor suppression mechanism. Also consistent with this hypothesis is the well-known observation that many cancers show an embryonic reversion such as the Warburg effect. The full identification of such molecular mechanisms would facilitate the invention of novel methods for modulating said molecular mechanisms in cells and tissue in vivo, to cause an “induced tissue regeneration” (iTR) to facilitate the repair of said tissues afflicted with trauma or degenerative disease, including but not limited to age-related degenerative disease, as well as facilitate basic research in tissue regeneration, to allow screens for agents capable of causing “induced tissue maturation” (iTM) in embryonic (i.e., pre-fetal or prenatal) cells for the purpose of making cells with a phenotype relatively more mature such as that of a fetal or adult phenotype, and for the identification and targeting of malignant or pre-malignant cells that have reverted to said embryonic phenotype for the purpose of diagnosis and therapy and maturing those cells to a more mature fetal or adult phenotype to arrest their growth and/or modulate apoptosis to control when said malignant cells are treated with an anticancer agent. Such diagnosis includes the determination of the extent to which carcinomas, adenocarcinomas, or sarcomas have reverted to an embryonic phenotype (embryo-onco phenotype), then treating a patient's cancer with agents appropriate to that phenotype, i.e. agents that are effective in inhibiting the replication or apoptosis of the cancer cells in that particular phenotype. In addition, the invention provides methods and compositions capable of causing “induced cancer cell maturation” (iCM), said agents have the potential to be therapeutic in an unusually broad spectrum of malignancies both as single agents and in combination with other therapeutic agents such as commonly used chemotherapeutic agents.

### SUMMARY

The present disclosure provides compounds, compositions, and methods useful for creating artificial intelligence software (deep learning) useful in identifying whether cells display an embryonic or a fetal or adult phenotype, the relative maturity of said fetal or adult cells, regulatory noncoding RNAs and mRNAs involved in the embryonic-fetal transition (EFT), the potential for tissue regeneration, and cancer, screening for and utilizing agents capable of modulating molecular pathways regulating the EFT in mammalian cells with a goal of causing iTR in cells and tissues not fully capable of such scarless regeneration, to screen for and utilizing agents capable of causing iTM in order to produce cells with an adult phenotype from cells previously in an embryonic or pre-fetal pattern of gene expression, and to detect and target malignant cells that have reverted to an embryonic phenotype in order to diagnose and treat cancer, and to screen for agents capable of causing iCM.

In one aspect of the present disclosure, deep learning algorithms are provided that allow researchers to identify patterns of gene expression in cells and tissues as belonging to embryonic or fetal, or adult sources. The algorithms describe a method of identification of the developmental status of an animal cell comprised of the steps of 1) collating RNA expression data from said cells, 2) training an artificial intelligence algorithm to identify where in the timeline of embryonic development said cells normally reside based on said RNA expression profile, and 3) testing RNA expression profiles to assign the cells from which the RNA was derived on the timeline with at least 90% accuracy.

In another aspect of the present disclosure, deep learning algorithms are provided that allow for the identification of genes differentially expressed in embryonic versus fetal or adult sources.

In another aspect, a series of screening criteria are described for determining whether candidate regulatory genes of the EFT are screened against RNA from developing mammals such as murine or human sources, or malignant counterparts of particular cell types to validate said genes as critical in the molecular pathways regulating EFT.

In another aspect, the present disclosure provides a method of identifying said genes, RNAs and proteins regulating the EFT in mammalian species, including primate species, more specifically, the human species, wherein said genes are identified by comparing the expression of genes that encode mRNAs and noncoding RNAs or splice variants in said RNAs that are differentially expressed in the embryonic stages of development compared to fetal and adult stages of development using RNA sequencing technology, gene expression array-based analysis of comparative pathway analysis, and the use of deep neural network analysis. More specifically, said methods identify genes encoding mRNAs and noncoding RNAs differentially expressed in multiple diverse somatic cell types in prenatal stages of development, specifically, the embryonic phases of development (before EFT) compared to fetal and adult cells (subsequent to EFT). In the case of the human species, the transition from embryonic to fetal development occurs at about 8 weeks of gestational development, in mouse it occurs at approximately 16 days, and in the rat species, at approximately 17.5 days (www.php.med.unsw.edu.au/embryology/index).

In another aspect of the disclosure, pluripotent stem cell-derived clonal, oligoclonal, pooled clonal, or pooled oligoclonal embryonic progenitor cell lines displaying gene expression patterns specific to the embryonic phase of mammalian development are utilized as a sourse of coding and noncoding RNAs and compared to the coding and noncoding RNAs in cells and tissues from fetal or adult-derived sources to identify genes encoding mRNAs, noncoding RNAs or splice variants, and transcriptome-based signaling pathway alterations in said fetal and adult cells compared to cells in the embryonic phases of development.

In another aspect of the disclosure, gene expression data from cells treated with small molecule drugs or other agents is analyzed to identify factors useful in inducing an embryonic pattern of gene expression in fetal or adult-derived cells leading to iTR, or alternatively, to induce the maturation of cells with an embryonic pattern of gene expression into corresponding cells with an adult pattern of expression (iTM or iCM).

In another aspect of the disclosure, mammalian cells are fetal or adult-derived cells are modulated in vitro to alter the EFT and the resulting cells are re-introduced into tissue in vivo to increase regenerative potential.

In another aspect of the disclosure, transcriptional regulatory genes differentially expressed in diverse types of somatic cells in the embryonic phases of development are compared to diverse types of somatic cells in phases of development after the embryonic phases such as adult cell types incapable of participating in TR, to identify those genes whose altered expression of alterations in splice variants is causative in the repression of tissue regeneration capacity in vivo in adult mammals. In some embodiments, methods of identifying genes whose expression or repression are capable of iTR comprises comparing the transcriptome of clonal, oligoclonal, or pooled clonal or pooled oligoclonal hPS cell-derived embryonic progenitor cell lines with the transcriptome of adult-derived cells or tissues of diverse types to identify genes commonly expressed in the embryonic progenitors or with RNA splice variants in the embryonic progenitors but not expressed or expressed at markedly lower levels in adult-derived cells, or alternatively, genes expressed in adult-derived cells or RNAs with splice variants, but not expressed or expressed at markedly lower levels in clonal, oligoclonal, or pooled clonal or pooled oligoclonal hPS cell-derived embryonic progenitor cell lines. In another embodiment, candidate iTR genes that are both expressed at higher levels in embryonic progenitor cells compared to adult-derived cells and which are also implicated in oncogenesis, or genes that are both expressed at lower levels in embryonic progenitor cells compared to adult-derived cells and which are also implicated in tumor suppression, are identified as candidate iTR genes.

In another aspect, the disclosure provides methods of optimizing the protocol for the administration of iTR factors, wherein the factors include those capable in other conditions of inducing pluripotency in somatic cell types, that is, in generating iPS cells, said factors including combinations of the genes: OCT4, SOX2, KLF4, NANOG, ESRRB, NR5A2, CEBPA, MYC, LIN28A and LIN28B, their encoded RNAs, or proteins, in diverse cell and tissue types to identify combinations of factors and/or repressors optimized for particular cell and tissue types.

In another aspect, the disclosure provides methods for the administration of iTR factors, wherein the factors include those capable in other conditions of inducing pluripotency in somatic cell types, that is, in generating iPS cells, said factors including combinations of the genes: OCT4, SOX2, KLF4, NANOG, ESRRB, NR5A2, CEBPA, MYC, TERT, LIN28A and LIN28B, their encoded RNAs, or proteins, in vitro to revert fetal or adult-derived cells to their embryonic counterpart or in vivo to induce tissue regeneration in diseased tissue without reverting the cells in said tissue to pluripotent stem cells.

In another aspect, the disclosure provides methods for the administration chemical inducers of iTR, wherein the inducers include those capable in other conditions of improving the efficiency of inducing pluripotency in somatic cell types, that is, in generating iPS cells, said factors including combinations of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, Inhibitors of DNA methyltransferase, activators of 3′ phosphoinositide-dependent kinase 1, promoters of glycolysis, RAR agonists, agents that mimic hypoxia, activators of telomerase, or inhibitors of the MAPK/ERK pathway, administered in vitro to revert fetal or adult-derived cells to their embryonic counterpart or in vivo to induce tissue regeneration in diseased tissue without reverting the cells in said tissue to pluripotent stem cells.

In another aspect, the disclosure provides methods of screening combinations of iTR genes in diverse cell and tissue types to identify combinations of factors and/or repressors optimized for particular cell and tissue types.

In another aspect, the disclosure provides methods of modifying the expression of iTR genes in cultured cells to restore them to a state wherein they are capable of participating in iTR when transplanted into a tissue otherwise incapable of undergoing sufficient TR. In another aspect of the disclosure, the telomerase catalytic component, including but not limited to the human gene TERT, is transiently expressed in combination with the iTR genes of the present disclosure in the target cells or tissues in which TR is to be induced, to extend the proliferative capacity of the somatic cells thereby facilitating TR. Said coexpression of telomerase activity with the iTR genes such as LIN28A, LIN28B, or clustered protocadherin genes of the alpha or beta cluster that are expressed at relatively higher levels in the embryonic state such as PCDHA4, PCDHB10, PCDHB2, PCDHB9, and, and is particularly useful in species with short telomeres and wherein cell replicative senescence occurs during the lifespan of that organism such as is the case in the human species. In another aspect of the disclosure, the telomerase catalytic component, including the human gene TERT is transiently expressed (as opposed to constitutively expressed) in the target cells or tissues to extend the proliferative capacity of the somatic cells without immortalizing the cells.

In another aspect of the disclosure, the telomerase catalytic component, including but not limited to the human gene TERT, is transiently expressed in combination with chemical inducers of iTR of the present disclosure in the target cells or tissues in which TR is to be induced, to extend the proliferative capacity of the somatic cells thereby facilitating TR. Said coexpression of telomerase activity with the chemical inducers of iTR such as combinations of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, Inhibitors of DNA methyltransferase, activators of 3′ phosphoinositide-dependent kinase 1, promoters of glycolysis, RAR agonists, agents that mimic hypoxia, activators of telomerase, or inhibitors of the MAPK/ERK pathway, is particularly useful in species with short telomeres and wherein cell replicative senescence occurs during the lifespan of that organism such as is the case in the human species. In another aspect of the disclosure, the telomerase catalytic component, including the human gene TERT is transiently expressed (as opposed to constitutively expressed) in the target cells or tissues to extend the proliferative capacity of the somatic cells without immortalizing the cells.

In another aspect, the present disclosure provides a means of engineering an animal model, preferably a mouse model capable of robust regenerative potential, said mouse being in a common laboratory strain of mice thereby facilitating molecular genetic and animal preclinical studies. Said robustly regenerating mouse is produced by creating mice that express iTR RNA transcripts or inhibit the transcription, stability, or translation of genes, RNAs, and proteins inhibiting iTR and breeding said mice together, and/or treating said mice, or specific tissue in said mice, with agents capable of causing global changes in iTR gene expression such that the profoundly regenerating mouse has a sufficient number of embryonic patterns of gene expression for the tissue regeneration desired.

In one embodiment, a treatment of aging and age-related disease is described.

Inducing heterochrony and modifying somatic development can generate larger or smaller animals or modify lifespans.

Heterochrony, or the alteration of the developmental timeline, has been selected in the case of numerous species to profoundly modify the resulting animal, occasionally producing “hopeful monsters.” Similarly, altering the RNA levels of the present disclosure are also capable of modulating developmental timing, body size in adulthood, as well as lifespan.

Numerous aspects of aging and age-related disease are presented in the present disclosure that can be addressed using iTR therapy. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; auditory disorders including hearing loss; and hematological disorders such as aplastic anemia and failed hematopoietic stem cell grafts.

In another aspect, the disclosure provides methods of modifying the expression of iTR genes in cells in vivo to restore them to a state wherein they are capable of participating in iTR.

In another aspect of the disclosure, methods are provided to cause iTR in tissues afflicted with degenerative disease including, but not limited to osteoarthritis wherein the means of effecting iTR in the diseased tissue utilizes a gene expression vector or vectors that cause the exogenous expression of the iTR genes disclosed herein including but not limited to a member of the LIN family such as LIN28A or LIN28B together with telomerase catalytic component, such as human TERT. In another aspect, the disclosure provides a method of identifying a candidate global modulator of TR activity comprising: (i) providing a composition comprising: (a) the candidate modulator or multiplicity of modulators of TR activity in a purified state or in a mixture with other molecules; (b) somatic cells not capable of TR wherein said cells express a fetal or adult pattern of gene expression as opposed to an embryonic pattern of gene expression; (c) a reporter construct present within the somatic cells or within extracts of said cells incapable of TR wherein the promoter of a gene differentially regulated in somatic cells in the embryonic phases of development compared to fetal and adult stages drives the expression of a reporter gene; and (ii) determining whether the candidate modulator or a multiplicity of modulators affect expression of the reporter gene, wherein altered expression of the reporter gene as compared with expression of the gene in the absence of the candidate modulator indicates that the compound modulates iTR activity.

In some embodiments, a method of identifying a candidate modulator of TR further comprises administering a candidate compound or multiplicity of compounds identified as modulators of TR to a subject.

In some embodiments, a method of identifying a candidate global modulator of TR further comprises administering a candidate compound for TR to cells derived from fetal or adult sources and assaying the expression COX7A1 through the use of an easily measured readout such as fluorescence generated from GFP driven by the COX7A1 promoter.

In some embodiments, a method of identifying a candidate modulator of TR further comprises administering a candidate compound for TR to cells derived from fetal or adult sources and assaying the expression of COX7A1 through the assay of the degree of methylation of the CpG island in the COX7A1 gene.

In some embodiments, a method of identifying a compound further comprises administering the compound to a subject. In some embodiments, the subject is a non-human animal, e.g., a non-human animal that serves as a model for TR or wound healing. In some embodiments, the subject is a human

In another aspect, the present disclosure provides a pharmaceutical composition comprising: (a) a modulator of iTR; and (b) a pharmaceutically acceptable carrier.

In another aspect, genes regulating the EFT are altered such that cancer cells are treated with agents that alter the expression of the genes from that of an embryonic state to that of a fetal or adult state to cause iCM. In another aspect, genes regulating the EFT are altered such that cancer cells are altered in the expression of the genes from that of an embryonic state to that of a fetal or adult state to cause iCM through the use of a histone deacetylase inhibitor.

Certain conventional techniques of cell biology, cell culture, molecular biology, microbiology, recombinant nucleic acid (e.g., DNA) technology, immunology, etc., which are within the skill of the art, may be of use in aspects of the disclosure. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3.sup.rd ed., Cold Spring Harbor Laboratory Press, Cold Spring Harbor, 2001; Harlow, E. and Lane, D.,

Antibodies—A Laboratory Manual, Cold Spring Harbor Laboratory Press, Cold Spring Harbor, 1988; Burns, R, Immunochemical Protocols (Methods in Molecular Biology) Humana Press; 3rd ed., 2005, Monoclonal antibodies: a practical approach (P. Shepherd and C Dean, eds., Oxford University Press, 2000); Freshney, R. I., “Culture of Animal Cells, A Manual of Basic Technique”, 5th ed., John Wiley & Sons, Hoboken, N.J., 2005). All patents, patent applications, websites, databases, scientific articles, and other publications mentioned herein are incorporated herein by reference in their entirety.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 Experimental design. Labelled transcriptomic data is used to train an ensemble of 20 deep neural networks with a single output neuron and a multiclass deep neural network with five neurons, one for each class to predict the “embryonic score” of the sample.

FIG. 2A through FIG. 2D show the classifier training and validation performance. Gene (FIG. 2A and FIG. 2B and pathway (FIG. 2C and FIG. 2D) level input data comes from Affymetrix (left panel) and Illumina (right panel) data with labeled cross validation. Presented are F1 scores obtained on training, internal validation and external validation sets (see Methods section for detailed description of nested cross-validation protocol employed).

FIG. 3A through FIG. 3D. Predicting embryonic state through DNN ensemble. (FIG. 3A) Validation confusion matrix performance for DNN ensemble trained on Illumina data. (FIG. 3B) Validation confusion matrix performance for DNN ensemble trained on Affymetrix data. (FIG. 3C) Expression level across 5 groups of key four genes obtained from top-15 of both GBM and DNN classifiers. (FIG. 3D) Embryonic scores obtained through Affymetrix and Illumina DNN ensembles for Affymetrix platform data set GSE65369 consisting of samples from different stages of neural lineage differentiation from ESC cells.

FIG. 4. Top 50 genes identified by DNN as differentially expressed in embryonic vs adult cell types

FIG. 5. Genes identified by DNN as differentially expressed in embryonic vs adult cell types with an FDR PValue <0.005.

FIG. 6A through FIG. 6D. RNA expression determined by RNA-seq in Cox7a1, Naaladl1, Plpp7, and Lin28b in whole mouse extracts during mouse development. Time points represent days post coitum (dpc).

FIG. 7A through FIG. 7D. RNA expression determined by Illumina gene expression bead arrays for COX7A1 (a), NAALADL1 (b), PLPP7 (c), and LIN28B (d) in human ES cells (ES), mean values in clonal embryonic progenitor cell lines (EP), early passage fibroblasts cultured in vitro from the upper arm of fetuses at differing time points in development, and early passage fibroblasts cultured in vitro from the upper arm of postnatal humans at differing ages, and adult-derived skin fibroblasts before and after (iPS Cells) transcriptional reprogramming to pluripotency.

FIG. 8A through FIG. 8D. RNA expression determined by Illumina gene expression bead arrays for COX7A1 (a), NAALADL1 (b), PLPP7 (c), and LIN28B (d) in human ES cells (ES), the clonal embryonic progenitor cell lines 4D20.8. E3, and SK5, analogous adult counterparts (MSCs, preadipocytes, and skeletal myoblasts respectively), and sarcomas corresponding to the aforementioned tissue types respectively.

FIG. 9. Inverse correlation of the embryonic marker LIN28B and the adult markers COX7A1 (a), PLPP7 (c), and NAALADL1 (b) in various sarcomas.

FIG. 10. shows novel markers of embryonic vs adult cells identified by RNA-sequencing.

FIG. 11. shows AMH expression in diverse embryonic vs adult cell types.

FIG. 12. shows LINC01021 expression in diverse embryonic vs adult cell types.

FIG. 13. shows RGPD1 expression in diverse embryonic vs adult cell types.

FIG. 14. shows ZNF300P1 expression in diverse embryonic vs adult cell types.

FIG. 15. shows LINC00654 expression in diverse embryonic vs adult cell types.

FIG. 16. shows PCDHGA12 expression in diverse embryonic vs adult cell types.

FIG. 17. shows reads determined by RNA-sequencing in the clustered protocadherin gene locus. RS-27 is an adult MSC cell line and RS-77 is an embryonic vascular endothelial progenitor cell line.

FIG. 18. shows agents capable of facilitating iTR discovered through screening using the COX7A1 marker. Values under cell lines represent proportional decreased values for COX7A1 transcript in the cell line.

FIG. 19. shows agents capable of facilitating iCM discovered through screening using the COX7A1 marker. Values under cell lines represent proportional increased values for COX7A1 transcript in the cell line.

FIG. 20. shows additional agents capable of facilitating iCM discovered through screening using the COX7A1 marker. Values under cell lines represent proportional increased values for COX7A1 transcript in the cell line.

FIG. 21. shows the formula used to calculate Embryonic score (ES).

FIG. 22. shows the multilayer neural network formula.

FIG. 23. shows methylation status of the fetal and adult-specific gene COX7A1 and PCDHB2 which is expressed at relatively higher levels in embryonic cells in the clonal embryonic progenitor (EP) cell lines 30MV2 and 4D20.8 and the analogous adult-derived human aortic endothelial cells (HAECs) and adult-derived mesenchymal stem cells (MSCs). The height of the histograms corresponds to the percent of methylated CpGs.

FIG. 24A through FIG. 24D shows RNA-sequencing of adult-derived skin fibroblasts (MDW) treated with diverse lentiviral constructs expressing the genes shown. Values shown for the transcript of LIN28A exogenously expressed in the cells together with the iTR markers COX7A1 and CAT, which were downregulated in a manner similar to though to a lesser extent than the cells completely reprogrammed to iPS cells. The gene GFER was upregulated in a manner proportional to LIN28A expression.

FIG. 25. shows expression of LIN28A and LIN28B in fetal liver-derived CD34+ hematopoietic stem cells and CD36+ erythroid progenitors compared to adult-derived bone marrow (BM) and peripheral blood (PB) blood cells of diverse types as assayed by Illumina gene expression bead array. Values >130 relative flurorescence units (RFUs) being considered positive, those <100 being considered negative.

FIG. 26A and FIG. 26B. shows expression of COX7A1 and LIN28B in control embryonic stem (ES) cells, normal adult mesenchymal stem cells (MSCs), normal blood cells, compared to diverse normal and malignant epithelia from bronchi, lung, epidermis, kidney, liver, breast, and dermal melanocytes as assayed by Illumina gene expression bead array. Values >130 relative flurorescence units (RFUs) being considered positive, those <100 being considered negative.

FIG. 27. shows expression of COX7A1 in normal adult-derived stromal cell types including: adult-derived human mesenchymal stem cells (hMSCs), normal human articular chondrocytes (NHAC), normal human diploid fibroblasts (NHDF), normal human osteoblasts (NHOst), and skeletal muscle cells (SkMC), as well as the diverse sarcoma lines shown. RFUs <75 are considered negative, values >90 are considered positive for expression.

FIG. 28. shows glycolytic stress test results. The undifferentiated embryonic lipogenic cells; namely, the clonal EP to white adipocytes designated E3, the adult-derived preadipocytes to subcutaneous adipose tissue (SAT), and the liposarcoma cell lines CRL3043 and CRL 3044 were exposed to a glycolytic stress test in parallel. Extracellular acidification rates (ECAR) are shown after glycolytic stresses.

FIG. 29. shows the percent confluency achieved by plating equal numbers of umbilical cord-derived and adult skin-derived stromal fibroblasts both of which expressed the fetal/adult marker COX7A1 after 80 hours of treatment with DMEM supplemented with 10% FBS (CTRL) or the same medium and serum additionally supplemented with either 10 ng/mL or 100 ng/mL of secreted GFER.

FIG. 30. shows Illumina gene expression bead array values for the expression of the fetal/adult marker COX7A1 and the fibrosis marker COL1A1 in MDW adult-derived skin fibroblasts in conditions of normal growth medium (DMEM supplemented with 10% FBS (Ctrl)), or the same medium supplemented with mRNA for cMYC or SOX2, or mRNA for cMYC or SOX2 supplemented with 0.5 mM valproic acid.

### DETAILED DESCRIPTION

### Abbreviations

AC—Adult-derived cells
AMH—Anti-Mullerian Hormone
ASC—Adult stem cells
cGMP—Current Good Manufacturing Processes
CM—Cancer Maturation
CNS—Central Nervous System
DMEM—Dulbecco's modified Eagle's medium
DMSO—Dimethyl sulphoxide
DNN—Deep Neural Network
DPBS—Dulbecco's Phosphate Buffered Saline
ED Cells—Embryo-derived cells; hED cells are human ED cells
EDTA—Ethylenediamine tetraacetic acid
EFT—Embryonic-Fetal Transition
EG Cells—Embryonic germ cells; hEG cells are human EG cells
EP—Embryonic progenitors
ES Cells—Embryonic stem cells; hES cells are human ES cells
ESC—Embryonic Stem Cells
FACS—Fluorescence activated cell sorting
FBS—Fetal bovine serum
FPKM—Fragments Per Kilobase of transcript per Million mapped reads from RNA sequencing.
GFER—Growth Factor, Augmenter of Liver Regeneration (ALR)
GFP—Green fluorescent protein
GMP—Good Manufacturing Practices
hED Cells—Human embryo-derived cells
hEG Cells—Human embryonic germ cells are stem cells derived from the primordial germ cells of fetal tissue.
HESC—Human Embryonic Stem Cells
hiPS Cells—Human induced pluripotent stem cells are cells with properties similar to hES cells obtained from somatic cells after exposure to hES-specific transcription factors such as SOX2, KLF4, OCT4, MYC, or NANOG, LIN28, OCT4, and SOX2.
HSE Human skin equivalents are mixtures of cells and biological or synthetic matrices manufactured for testing purposes or for therapeutic application in promoting wound repair.
iCM Induced Cancer Maturation.
iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.
iTM—Induced Tissue Maturation
iTR—Induced Tissue Regeneration
MEM—Minimal essential medium
MSCs—Mesenchymal stem cells
NT—Nuclear Transfer
PBS—Phosphate buffered saline
PS fibroblasts—Pre-scarring fibroblasts are fibroblasts derived from the skin of early gestational skin or derived from ED cells that display a prenatal pattern of gene expression in that they promote the rapid healing of dermal wounds without scar formation.
RFU—Relative Fluorescence Units
RNA-seq—RNA sequencing
SFM—Serum-Free Medium
TR—Tissue Regeneration

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application No. 60/332,510, filed Nov. 26, 2001; Ser. No. 10/304,020, filed Nov. 26, 2002; PCT application no. PCT/US02/37899, filed Nov. 26, 2003; U.S. application No. 60/705625, filed Aug. 3, 2005; U.S. application No. 60/729173, filed Aug. 20, 2005; U.S. application no. 60/818813, filed Jul. 5, 2006, PCT/US06/30632, filed Aug. 3, 2006, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “cancer maturation” refers to the alteration of gene expression in premalignant or malignant cancer cells such that said premalignant or malignant cancer cells that initially express markers of embryonic cells, are altered to express markers of fetal or adult cells.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “cellular reconstitution” refers to the transfer of a nucleus of chromatin to cellular cytoplasm so as to obtain a functional cell.

The term “clonal” refers to a population of cells obtained the expansion of a single cell into a population of cells all derived from that original single cells and not containing other cells.

The term “colony in situ differentiation” refers to the differentiation of colonies of cells (e.g., hES, hEG, hiPS, hEC or hED) in situ without removing or disaggregating the colonies from the culture vessel in which the colonies were propagated as undifferentiated stem cell lines. Colony in situ differentiation does not utilize the intermediate step of forming embryoid bodies, though embryoid body formation or other aggregation techniques such as the use of spinner culture may nevertheless follow a period of colony in situ differentiation.

The term “cytoplasmic bleb” refers to the cytoplasm of a cell bound by an intact or permeabilized but otherwise intact plasma membrane, but lacking a nucleus.

The term “differentiated cells” when used in reference to cells made by methods of this disclosure from pluripotent stem cells refer to cells having reduced potential to differentiate when compared to the parent pluripotent stem cells. The differentiated cells of this disclosure comprise cells that could differentiate further (i.e., they may not be terminally differentiated).

The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. (www.php.med.unsw.edu.au/embryology/index.php?title=Mouse_Timeline_Detailed). The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “global modulator of TR” or “global modulator of iTR” refers to agents capable of modulating a multiplicity of iTR genes or iTM genes including, but not limited to, agents capable of downregulating COX7A1 while simultaneously up-regulating PCDHB2, or down-regulating NAALADL1 while simultaneously up-regulating AMH in cells derived from fetal or adult sources and are capable of inducing a pattern of gene expression leading to increased scarless tissue regeneration in response to tissue damage or degenerative disease.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell lines (see, e.g., U.S. Pat. Nos. 7,582,479; 7,217,569; 6,887,706; 6,602,711; 6,280,718; and 5,843,780 to Thomson). The hED cells may be derived from preimplantation embryos produced by fertilization of an egg cell with sperm or DNA, nuclear transfer, or chromatin transfer, an egg cell induced to form a parthenote through parthenogenesis, analytical reprogramming technology, or by means to generate hES cells with hemizygosity or homozygosity in the HLA region. The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA.

The term “human embryonic stem cells” (hES cells) refers to human ES cells.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced Cancer Maturation” refers to methods resulting in a change in the phenotype of premalignant or malignant cells such that subsequent to said induction, the cells express markers normally expressed in that cell type in fetal or adult stages of development as opposed to the embryonic stages.

The term “induced tissue regeneration” refers to the use of the methods of the present disclosure to alter the molecular composition of fetal or adult mammalian cells such that said cells are capable or regenerating functional tissue following damage to that tissue wherein said regeneration would not be the normal outcome in animals of that species.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iCM factors” refers to molecules that alter the levels of CM activators and CM inhibitors in a manner leading to CM in a tumor for therapeutic effect.

The term “iCM genes” refers to genes that when altered in expression can cause CM in a tumor for therapeutic effect. The term “iTR factors” refers to molecules that alter the levels of TR activators and TR inhibitors in a manner leading to TR in a tissue not naturally capable of TR.

The term “iTR genes” refers to genes that when altered in expression can cause induced tissue regeneration in tissues not normally capable of such regeneration.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments, a nucleic acid comprises standard nucleosides (abbreviated A, G, C, T, U). In other embodiments, a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant disclosure. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid are modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the disclosure encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or concensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the disclosure provides embodiments relating to precursor polypeptides and embodiments relating to mature versions of a polypeptide.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art.

siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the disclosure. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precurors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound described in the disclosure).

The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present disclosure would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “TR activator genes” refers to genes whose lack of expression in fetal and adult cells but whose expression in embryonic phases of development facilitate TR.

The term “TR inhibitor genes” refers to genes whose expression in fetal and adult animals inhibit TR.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant disclosure is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments, amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances larger domains may be removed without substantially affecting function. In certain embodiments of the disclosure the sequence of a variant can be obtained by making no more than a total of 5, 10, 15, or 20 amino acid additions, deletions, or substitutions to the sequence of a naturally occurring enzyme. In some embodiments, no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6× His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6.times.His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the disclosure a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the disclosure relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

In addition to the methods described below, methods that find use in the production and use of cells with an embryonic pattern of gene expression corresponding with scarless regenerative potential can be found in the following: PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, the disclosures of which are incorporated by reference in their entirety), each of which is incorporated by reference herein in its entirety.

### Design of Deep Learning Agorithm: Data Collection and Integration

Microarray data from diverse platforms can be generated or downloaded from Gene Expression Omnibus (GEO) and ArrayExpress. Gathered samples may, by way of nonlimiting examples, belong to one of the following broad cell classes: embryonic stem cell (ESC), induced pluripotent stem cell (iPSC), embryonic progenitor cells (EPC), adult stem cells (ASC) and diverse adult cell types (AC). By way on nonlimiting example, samples may include data obtained from the following microarray platforms: Illumina HumanHT-12 V4.0 (GPL10558), Illumina HumanHT-12 V3.0 (GPL6947), Affymetrix HT Human Genome U133A Array (GPL3921), Affymetrix GeneChip Human Genome U133 Array Set HG-U133A (GPL4557), Affymetrix Human Exon 1.0 ST Array (GPL5188), Affymetrix Human Genome U133 Plus 2.0 Array (GPL570), Affymetrix Human Genome U133A 2.0 Array (GPL571), Affymetrix Human Gene 1.0 ST Array (GPL6244), Affymetrix Human Genome U133A Array (GPL96), Affymetrix Human Genome U133 Plus 2.0 Array (GPL11670), or RNA-seq data.

### Data Processing

Separate processing pipelines for Affymetrix and Illumina data may be utilized. For each dataset, probe data can be extracted from raw files, and then converted to gene expression values. For the processing of Affymetrix-related datasets the Frozen RMA (fRMA) method can be utilized (McCall, M. N., et al, Frozen robust multiarray analysis (fRMA). Biostatistics 11, 242-253 (2010); McCall, M. N., et al, The Gene Expression Barcode: leveraging public data repositories to begin cataloging the human and murine transcriptomes. Nucleic Acids Res. 39, D1011-5 (2011); McCall, M. N., et al, Assessing affymetrix GeneChip microarray quality. BMC Bioinformatics 12, 137 (2011); McCall, M. N., et al, fRMA ST: frozen robust multiarray analysis for Affymetrix Exon and Gene ST arrays. Bioinformatics 28, 3153-3154 (2012)), which allows one to analyze microarrays individually or in small batches and then combine the data for analysis. For Illumina data, non-normalized files can be used with subsequent quantile normalisation.

After obtaining probe expression data, it can be converted to gene expression using annotation tables, available from GEO for Illumina platforms and ‘AnnotationDbi’ package from Bioconductor for Affymetrix platforms. Such tables contain probe-gene mapping for particular microarray platform. If multiple probes are mapped to same gene, geometric mean to average their signals can be utilized. After converting to genes, whole dataset (separately for Affymetrix and Illumina platforms) can be processed with quantile normalization algorithm. The samples to be classified can be normalized using the same set of quantiles as were determined for training dataset. Genes contained in every target platform set (Affymetrix and Illumina) can be used as input features for each classifier. Several machine learning methods can be compared for their performance

### Pathway Analysis.

For pathway level analysis each case sample group can be independently analyzed using an algorithm called OncoFinder (Buzdin, A. A. et al. Oncofinder, a new method for the analysis of intracellular signaling pathway activation using transcriptomic data. Front. Genet. 5, 55 (2014)). Taking the preprocessed gene expression data as an input, it allows for cross-platform dataset comparison with low error rate and has the ability to obtain functional features of intracellular regulation using mathematical estimations. For each investigated sample group it performs a case-reference comparison using Student's t-test and generates the list of significantly differentially expressed genes and calculates the Pathway Activation Strength (PAS), a value which serves as a qualitative measure of pathway activation. Positive and negative PAS values indicate pathway up- and down-regulation, respectively.

K-Nearest Neighbors Algorithm (kNN):

K-nearest neighbors algorithm is a simple non-parametric method, that can be applied to regression. The underlying idea of the method is to predict a value of a given object as an average of the values of its k nearest neighbors. The choice of optimal k is defined by the properties of the data. In the present invention, the scikit-learn implementation of the method can be utilized (Pedregosa, F. et al. Scikit-learn: Machine Learning in Python. J. Mach. Learn. Res. 12, 2825-2830 (2011)). Hyperparameters are tuned where the number of neighbors to use are (5-20), the neighbor weighting (uniform of inversely proportional to their distance), and metric (Manhattan, Euclidean, or Minkowski with p=3).

### Logistic Regression (LR).

Logistic regression is a widely used straightforward approach to model the dependence of a given variable Y on a set of independent variables X,. In the present invention the scikit-learn implementation is used (Pedregosa, F. et al. Scikit-learn: Machine Learning in Python. J. Mach. Learn. Res. 12, 2825-2830 (2011)). First, the data dimensionality is reduced using Principal Component Analysis with whitening, and then trained multiclass classifier with L₂-regularization. Hyperparameters tuned were the number of principal components (100-500), and regularization strength (0.1-100).

### Support Vector Machines (SVM).

SVM is another classical machine learning algorithm, which, in its basic form, constructs a set of hyperplanes separating multidimensional data into classes. The use of non-linear kernels allows SVM to perform non-linear classification. In the current study we used the scikit-learn implementation of the method (Edgar, R. Gene Expression Omnibus: NCBI gene expression and hybridization array data repository. Nucleic Acids Res. 30, 207-210 (2002)). Hyperparameters tuned were the type of kernel (linear, sigmoid, 3rd-degree polynomial, and radial basis function (Gaussian) kernels), and regularization strength (0.1-100).

### Gradient Boosting Machines (GBM).

Gradient boosting is a machine learning method used for classification and regression problems. This method uses an ensemble of weak models, like classification trees in this case, to generate predictions. We used XGBoost library (Kolesnikov, N. et al. ArrayExpress update—simplifying data submissions. Nucleic Acids Res. 43, D1113-6 (2015)) to implement gradient boosting classifier. Hyperparameters tuned were the number of trees grows (10-100), maximal depth of each tree (3-8), subsampling ratio (0.5-1.0), regularization parameters gamma (further partitioning threshold, 0.5-1) and minimal child weight (1-5), and step size shrinkage (0.005-0.05).

### Multiclass Deep Neural Network (DNN).

The number of input layer neurons was equal to the number of genes used. Hyperparameters tuned were the number of hidden layers (2-4), the number of neurons in each hidden layer (100-500), activation function for all layers except output one (ReLU, sigmoid, or tanh), L₂ weight-regularization strength (0.01 to 0.05), and dropout value (0.0 to 0.5). Output layer uses softmax activation. The neural network was trained for 200 epochs using Adam optimizer (McCall, M. N., Bolstad, B. M. & Irizarry, R. A. Frozen robust multiarray analysis (fRMA). Biostatistics 11, 242-253 (2010)).

### Ensemble of Deep Neural MNetworks (DNN Ens.).

The design of each network is similar to multiclass network, except output layer has only one neuron with sigmoid activation. Since running hyperparameter optimization for DNN ensemble is very computationally expensive, each network used the set of hyperparameters identified as optimal for multiclass network: 2 layers of 200 neurons, ReLU activation, 0.2 dropout, and 0.03 L₂ weight regularization strength. We trained 20 binary networks for each target platform set (Affymetrix and Illumina) to perform pairwise (one-vs-one) classification. Then we evaluated the overall ensemble vote for each class as the sum of four one-vs-one networks, which perform pairwise distinction of this class from four other classes.

### Training Classifiers

In order to use any of neural networks described above we need to train it on chosen datasets. To do this, we employed the following scheme.

First we preprocess the datasets (gathered from public data repositories, as well as the one provided by BioTime, Inc.) to convert probe data into genes, and apply quantile normalization.

Afterwards, we employ nested cross validation approach to tune hyperparameters and obtain unbiased estimation of classifier performance. Both outer and inner loops use stratified labeled 3-fold cross validation, with samples from same dataset belonging to either training or validation set, but not both.

In outer loop, we hold out a part of the data, and use the remaining samples to optimize classifier hyperparameters. We then verify that hyperparameters were not overfit by training classifier with found optimal hyperparameters, and testing it on the held out data. The hyperparameter tuning is repeated for each fold. This result is designated “Ext. validation”.

We use Tree of Parzen Estimators (TPE) algorithm (as implemented in hyperopt package (Bergstra, J., Yamins, D. & Cox, D. D. Hyperopt: A Python library for optimizing machine learning algorithms; SciPy 2013. in Proceedings of the 12th Python In Science Conference 13-20 (2013))) to optimize hyperparameters. For each parameter set it attempts, we run 3-fold cross validation, and use mean validation score as optimization target. For best hyperparameter set, we present its mean performance on training (“Training”) and validation (“Int. validation”) sets in internal cross validation loop.

Only training and validation scores for DNN ensemble are presented, since we do not run hyperparameter estimation for it due to high computational cost.

TABLE 1

| Method | Affymetrix | Illumina |
|---|---|---|
| kNN | k = 10, distance weighting, | k = 5, distance weighting, |
| | p = 3 | p = 2 |
| LR | 200 components, C = 0.34 | 200 components, C = 0.24 |
| SVM | Linear kernel, C = 0.48 | RBF kernel, C = 99.94 |
| GBM | 30 trees, depth = 5, | 80 trees, depth = 6, |
| | subsample 0.8, gamma = 0.6, | subsample 0.5, gamma = 1.0, |
| | min_child_weight = 2, | min_child_weight = 3, |
| | eta = 0.005 | eta = 0.05 |
| DNN | 2 hidden layers, 100 neurons | 2 hidden layers, 200 neurons |
| | per layer, ReLU activation, | per layer, ReLU activation, |
| | dropout 0.2, L₂ 0.03 | dropout 0.0, L₂ 0.04 |

### Determining a Sample's Embryonic Score

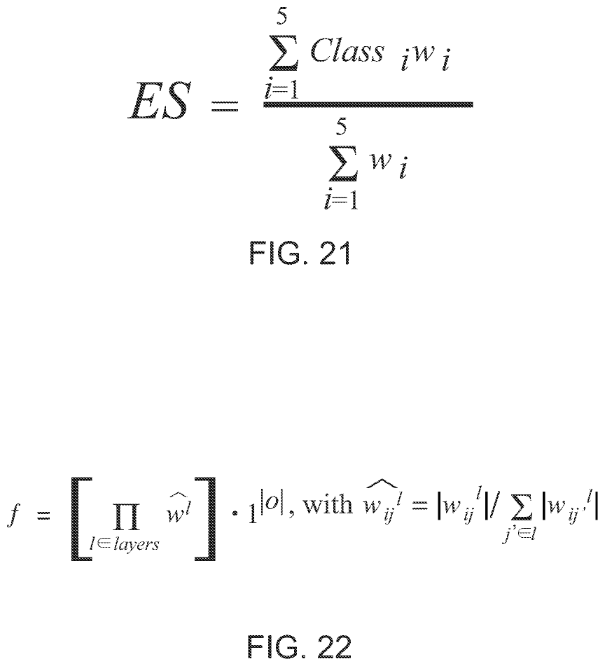

To determine how close the sample is to the embryonic state we use an ensemble of deep neural network predictors, built upon one of proposed approaches. The sample to be classified is subjected to same preprocessing protocol as training samples from appropriate platform. The genes are supplied to trained deep neural network predictors' input. Ensemble produces five scores—one for each class—which we use to calculate the Embryonic Score (ES) as shown in the formula shown in FIG. 21 where Class₁₋₅ is the predictor's output for each class, and w₁₋₅—are arbitrary degrees of embryonic development for chosen classes (we assign w_{ESC}=1.0, w_{iPSC}=0.9, W_{EPC}=0.7, W_{ASC}=0.5, W_{AC}=0.0).

As a result, the system outputs calculated the embryonic score for each sample.

In order to find out what genes are good markers of each stage of cell development, we used method proposed in (Yacoub, M. & Bennani, Y. HVS: A Heuristic for Variable Selection in Multilayer Artificial Neural Network Classifier. in Intelligent Engineering Systems Through Artificial Neural Networks 527-532 (1997)) that allows estimation of each feature importance directly from DNN's weight matrices. This method measures the magnitude with which every input feature is propagated all the way to output layer. For multilayer neural network, the expression can be written as shown in FIG. 22., where w′ is DNN weight matrix for layer 1, w₁′ is the connection weight value between neurons i and j on layer l, 1^{|O|} is the vector of all ones the size of output layer, and fis the vector of computed input feature importances.

For verification, we measured gene importance from trained multiclass GBM classifier by measuring how many times a particular feature is used to split a tree (f-score).

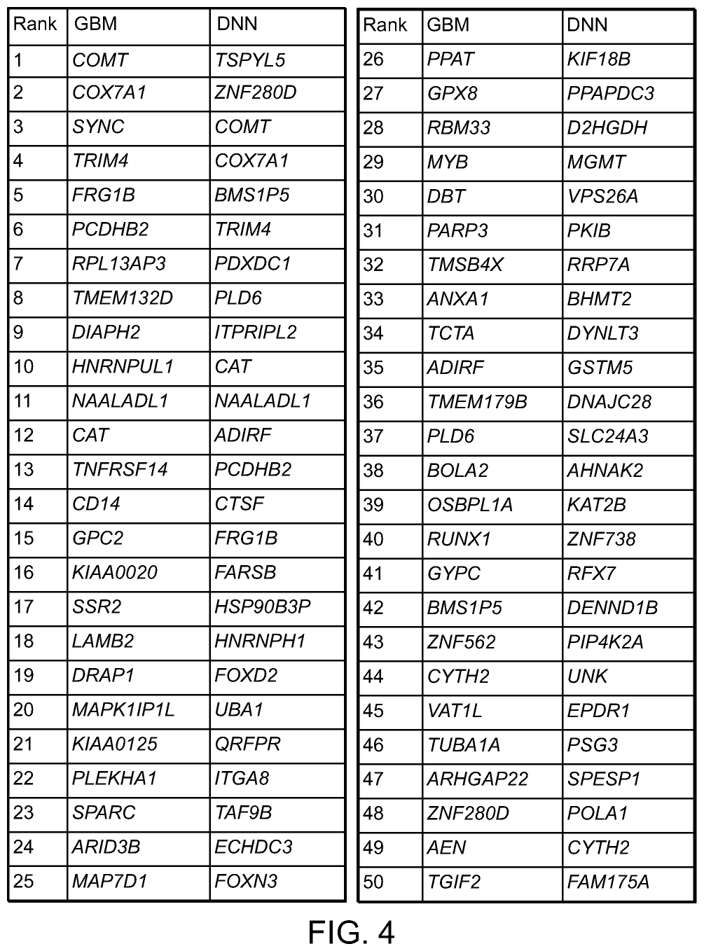

We found a significant overlap between important genes as scored by GBM or DNN (FIG. 4), which shows that both methods reply to large extent on the similar set of genes to make prediction.

### RNAi

By way of nonlimiting example, dsRNA was prepared from in vitro transcription reactions (Promega) using PCR-generated templates with flanking T7 promoters, purified by phenol extraction and ethanol precipitation, and annealed after resuspension in water. Intact experimental animals are injected with 4× 30 nL dsRNA on three consecutive days following induced tissue injury beginning with the first injection two hours after surgery.

### TR Modulation and iTR Modulators

The present disclosure provides novel iTR modulators and methods of use thereof. In some aspects, the invention provides novel methods of enhancing regeneration comprising administering an agent that alters the concentration of said iTR modulators to a multicellular organism in need thereof.

The applicants teach that primitive animals that display the potential for profound TR such as the regeneration of amputated limbs in axolotls, the regeneration of skin in MRL or the African Spiny Mouse, or the regeneration of whole body segments in planaria, do so by simply recapitulating normal embryonic development of the respective tissues. Furthermore, the applicants teach that the cause of inability to regenerate damaged tissue in TR-resistant mammals such as most murine species and humans is that certain embryonic gene transcription is altered in the EFT in these TR-resistant animals. The applicants further teach that the restoration of certain of these embryo-specific patterns of gene expression altered in the EFT in TR-resistant animals can induce competency for regeneration in any tissue, including responsiveness to organizing center factors, leading to complex tissue regeneration and a comcommitant reduction in scar formation. Lastly, the applicants teach novel agents and associated methods of inducing TR in mammalian species. Said methods facilitate TR in mammalian species in vivo, particularly in the species Homo sapiens.

Genes whose expression in fetal and adult animals inhibit TR are herein designated “TR inhibitors”, and genes whose lack of expression in fetal and adult cells but whose expression in embryonic phases of development facilitate TR are herein designated “TR activators.” Collectively, TR inhibitor genes and TR activator genes are herein designated iTR genes. Molecules that alter the levels of TR activators and TR inhibitors in a manner leading to TR are herein designated “iTR factors”. iTR genes and, the protein products of iTR genes, are often conserved in animals ranging from sea anemones to mammals The gene-encoded protein sequences, and sequences of nucleic acids (e.g., mRNA) encoding genes referred to herein, including those from from a number of different non-human animal species are known in the art and can be found, e.g., in publicly available databases such as those available at the National Center for Biotechnology Information (NCBI) (www.ncbi.nih.gov).

The TR inhibitory gene COX7A1 was observed to be expressed primarily in stromal as opposed to epithelial cells in normal tissue, though it was also expressed at lower levels in epithelial cultures. In the case of neoplasms, the gene was observed to be down-regulated in many stromal cancers such as osteosarcoma, chondrosarcoma, rhabdomyosarcoma, as well as some gliomas, carcinomas, and adenocarcinomas. This is consistent with the observation of increased glycolysis in cancer known as the Warburg effect, though the absence of COX7A1 expression has not previously been implicated in the Warburg effect. Since the applicants propose that TR genes are altered in the transition from embryonic to fetal development in part to prevent cancer in the adult, the repression of COX7A1 in stromal and some CNS and epithelial tumors would revert a stromal cell to an embryonic state, thereby facilitating oncogenesis. The exogenous induction of expression of COX7A1 in such tumors lacking expression would therefore have a therapeutic effect, in part by altering the activity of p53 and HIF1 alpha, and thereby inhibiting cell proliferation and increasing apoptosis in cancer cells.

In another embodiment, the present invention provides a means of detecting cancer cells. Rarely have researchers identified a marker of an abnormality associated with a majority of cancer cell types. As described herein, the markers distinguishing embryonic from their fetal and adult counterparts can be used to distinguish normal cells displaying an adult pattern of expression from malignant cells which display an embryonic pattern. Said detection methods, include but are not limited to detection of the expression of COX7A1, NAALADL1, AMH, and genes from the alpha, beta, and gamma clustered protocadherin genes including but not limited to PCDHA4, PCDHB2, and PCDHGA12 in an embryonic as opposed to fetal/adult pattern. This is useful not only in identifying malignant cells (with the exception of blood cells), but is also useful in identifying tumors that will be resistant to commonly-used chemotherapeutic agents which are characterized by their expression of a fetal/adult pattern.

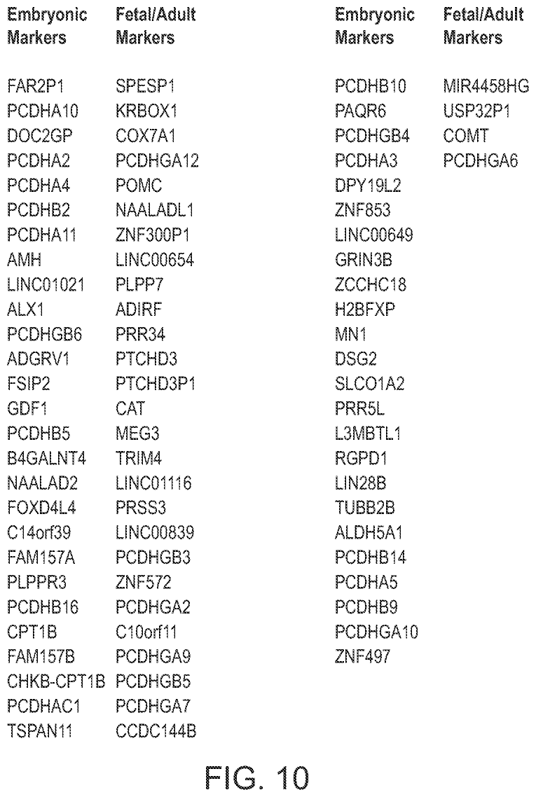

The disclosure provides a number of different methods of modulating iTR genes and a variety of different compounds useful for modulating iTR genes. In general, an iTR factor can be, e.g., a small molecule, nucleic acid, oligonucleotide, polypeptide, peptide, lipid, carbohydrate, etc. In some embodiments of the invention, iTR factors inhibit by decreasing the amount of TR inhibitor RNA produced by cells and/or by decreasing the level of activity of TR inhibitor genes. In the case of targeting TR inhibitors, factors are identified and used in research and therapy that reduce the levels of the product of the TR inhibitor gene. Said TR inhibitor gene can be any one or combination of TR inhibitor genes listed in FIG. 10 under the heading of “Fetal/Adult Markers”. The amount of TR inhibitor gene RNA can be decreased by inhibiting synthesis of TR inhibitor RNA synthesis by cells (also referred to as “inhibiting TR inhibitor gene expression”), e.g., by reducing the amount of mRNA encoding TR inhibitor genes or by reducing translation of mRNA encoding TR inhibitor genes. Said factor can be by way of nonlimiting example, RNAi targeting a sequence within the TR inhibitor genes listed in FIG. 10 under the heading of “Fetal/Adult Markers”.

In some embodiments, TR inhibitor gene expression is inhibited by RNA interference (RNAi). As known in the art, RNAi is a process in which the presence in a cell of double-stranded RNA that has sequence correspondence to a gene leads to sequence-specific inhibition of the expression of the gene, typically as a result of cleavage or translational repression of the mRNA transcribed from the gene. Compounds useful for causing inhibition of expression by RNAi (“RNAi agents”) include short interfering RNAs (siRNAs), short hairpin RNAs (shRNAs), microRNAs (miRNAs), and miRNA-like molecules.

One of skill in the art can readily design sequences for RNAi agents, e.g., siRNAs, useful for inhibiting expression of mammalian TR inhibitor genes, e.g., human TR inhibitor genes once one has identified said TR inhibitor genes. In some embodiments, such sequences are selected to minimize “off-target” effects. For example, a sequence that is complementary to a sequence present in TR inhibitor gene mRNA and not present in other mRNAs expressed in a species of interest (or not present in the genome of the species of interest) may be used. Position-specific chemical modifications may be used to reduce potential off-target effects. In some embodiments, at least two different RNAi agents, e.g., siRNAs, targeted to TR inhibitor gene mRNA are used in combination. In some embodiments, a microRNA (which may be an artificially designed microRNA) is used to inhibit TR inhibitor gene expression.

In some embodiments of the invention, TR inhibitor gene expression is inhibited using an antisense molecule comprising a single-stranded oligonucleotide that is perfectly or substantially complementary to mRNA encoding TR inhibitor genes. The oligonucleotide hybridizes to TR inhibitor gene mRNA leading, e.g., to degradation of the mRNA by RNase H or blocking of translation by steric hindrance. In other embodiments of the invention, TR inhibitor gene expression is inhibited using a ribozyme or triplex nucleic acid.

In some embodiments, of the invention, a TR inhibitor inhibitor inhibits at least one activity of an TR inhibitor protein. TR inhibitor activity can be decreased by contacting the TR inhibitor protein with a compound that physically interacts with the TR inhibitor protein. Such a compound may, for example, alter the structure of the TR inhibitor protein (e.g., by covalently modifying it) and/or block the interaction of the TR inhibitor protein with one or more other molecule(s) such as cofactors or substrates. In some embodiments, inhibition or reduction may be a decrease of at least about 5%, 10%, 15%, 20%, 25%, 30%, 35%, 40%, 45%, 50%, 55%, 60%, 65%, 70%, 75%, 80%, 85%, 90%, 95%, or 99% of a reference level (e.g., a control level). A control level may be the level of the TR inhibitor that occurs in the absence of the factor. For example, an TR factor may reduce the level of the TR inhibitor protein to no more than 95%, 90%, 85%, 80%, 75%, 70%, 65%, 60%, 55%, 50%, 40%, 30%, 25%, 20%, 10%, or 5% of the level that occurs in the absence of the factor under the conditions tested. In some embodiments, levels of the TR inhibitor are reduced to 75% or less of the level that occurs in the absence of the factor, under the conditions tested. In some embodiments, levels of the TR inhibitor are reduced to 50% or less of the level that occurs in the absence of the TR factor, under the conditions tested. In some embodiments, levels of the TR inhibitor are reduced to 25% or less of the level that occurs in the absence of the iTR factor, under the conditions tested. In some embodiments, levels of the TR inhibitor are reduced to 10% or less of the level that occurs in the absence of the iTR factor, under the conditions tested. In some cases the level of modulation (e.g., inhibition or reduction) as compared with a control level is statistically significant. As used herein, “statistically significant” refers to a p-value of less than 0.05, e.g., a p-value of less than 0.025 or a p-value of less than 0.01, using an appropriate statistical test (e.g, ANOVA, t-test, etc.).

In some embodiments of the invention, a compound directly inhibits TR inhibitor proteins, i.e., the compound inhibits TR inhibitor proteins by a mechanism that involves a physical interaction (binding) between the TR inhibitor and the iTR factor. For example, binding of a TR inhibitor to an iTR factor can interfere with the TR inhibitor's ability to catalyze a reaction and/or can occlude the TR inhibitors active site. A variety of compounds can be used to directly inhibit TR inhibitors. Exemplary compounds that directly inhibit TR inhibitors can be, e.g., small molecules, antibodies, or aptamers.

In some embodiments of the invention, an iTR factor binds covalently to the TR inhibitor. For example, the compound may modify amino acid residue(s) that are needed for enzymatic activity. In some embodiments, an iTR factor comprises one or more reactive functional groups such as an aldehyde, haloalkane, alkene, fluorophosphonate (e.g., alkyl fluorophosphonate), Michael acceptor, phenyl sulfonate, methylketone, e.g., a halogenated methylketone or diazomethylketone, fluorophosphonate, vinyl ester, vinyl sulfone, or vinyl sulfonamide, that reacts with an amino acid side chain of TR inhibitors. In some embodiments, an iTR factor inhibitor comprises a compound that physically interacts with a TR inhibitor, wherein the compound comprises a reactive functional group. In some embodiments, the structure of a compound that physically interacts with the TR inhibitor is modified to incorporate a reactive functional group. In some embodiments, the compound comprises a TR inhibitor substrate analog or transition state analog. In some embodiments, the compound interacts with the TR inhibitor in or near the TR inhibitor active site.

In other embodiments, an iTR factor binds non-covalently to a TR inhibitor and/or to a complex containing the TR inhibitor and a TR inhibitor substrate. In some embodiments, an iTR factor binds non-covalently to the active site of a TR inhibitor and/or competes with substrate(s) for access to the TR inhibitor active site. In some embodiments, an iTR factor binds to the TR inhibitor with a K_{d} of approximately 10⁻³ M or less, e.g., 10 ⁻⁴M or less, e.g., 10⁻⁵ M or less, e.g., 10⁻⁶ M or less, 10⁻⁷ M or less, 10⁻⁸ M or less, or 10⁻⁹ M or less under the conditions tested, e.g., in a physiologically acceptable solution such as phosphate buffered saline. Binding affinity can be measured, e.g., using surface plasmon resonance (e.g., with a Biacore system), isothermal titration calorimetry, or a competitive binding assay, as known in the art. In some embodiments, the inhibitor comprises a TR inhibitor substrate analog or transition state analog.

In the case of increasing the activity of TR activators, any one of combination of the TR activator genes listed in FIG. 10 under the heading “Embryonic Markers” may be used. The levels of the products of these genes may be introduced using the vectors described herein. In other embodiments, the iTR factors are constructs that introduce RNA into cells either directly or through gene expression constructs that are capable of inducing pluripotency if allowed to react with cells for a sufficient period of time, but for lesser times can cause iTR. Preferably, the RNAs do not include all of the RNAs needed for reprogramming to pluripotency and instead include only LIN28A or LIN28B optionally together with an agent to increase telomere length such as RNA for the catalytic component of telomerase (TERT). Most preferably, the agents to induce iTR are genes/factors induced by LIN28A or LIN28B-encoded proteins such as GFER, optionally in combination with an agent that increases telomere length such as the RNA or gene encoding TERT, and/or in combination with the factors disclosed herein important for iTR such as 0.05-5 mM valproic acid, preferably 0.5 mM valproic acid, 1-100 ng/mL AMH, preferably 10 ng/mL AMH, and 2-200 ng/mL GFER, preferably 20 ng/mL. When administered in vivo, such factors are preferably administered in a slow-release hydrogel matrix such as one comprised of chemically modified and crosslinked hyaluronic acid and collagen such as HyStem matrices.

### Reporter-Based Screening Assays for iTR Factors

The invention provides methods for identifying iTR factors using (a) a reporter molecule comprising a readily-detectable marker such as GFP or beta galactosidase whose expression is driven by the promoter of one of the TR activator genes described herein such as that for COX7A1. The invention provides screening assays that involve determining whether a test compound affects the expression of TR activator genes and/or inhibits the expression of TR inhibitory genes. The invention further provides reporter molecules and compositions useful for practicing the methods. In general, compounds identified using the inventive methods can act by any of mechanism that results in increased or decreased TR activator or inhibitor genes respectively. In the case of the COX7A1 promoter, a promoter sequence flanking the 5′ end of the human gene has been characterized to the position of −756 bases to the ATG translation start codon (Yu, M., et al. Biochimica and Biophysica Acta 1574 (2002) 345-353). Transcription start site of the most cDNAs were observed to be at −55 bases of the translation start codon.

The promoter, as well as the rest of the gene sequence, lays in a CpG island, similarly to the promoters of many housekeeping genes, although the expression of COX7A1 is tissue specific. CpG islands are characterized by the abundance of CG dinucleotides that surpasses that of the average, expected content for the genome, over the span of at least 200 bases. The promoter comprises several regulatory binding site sequences: MEF2 at position −524, as well as three E boxes (characterized as E1, E2, and E3), at, respectively −positions −58, −279 and −585; E box is a DNA binding site (CAACTG) that binds members of the myogenic family of regulatory proteins. Additionally, in the region approximately −95 to −68 bases, there are multiple CG rich segments similar to the one recognized by the transcription factor Sp1.

The gene itself, as characterized in GRCh38.p7 primary assembly, occupies 1948 bases between positions 36150922 and 36152869 on Human chromosome 18, and comprises 4 exons interspersed by three introns. Gene sequence, with the promoter sequence is curated at NCBI under locus identifier AF037372.

### Reporter Molecules, Cells, and Membranes

In general, detectable moieties useful in the reporter molecules of the invention include light-emitting or light-absorbing compounds that generate or quench a detectable fluorescent, chemiluminescent, or bioluminescent signal. In some embodiments, activation of TR activator genes or inhibition of TR inhibitory genes causes release of the detectable moiety into a liquid medium, and the signal generated or quenched by the released detectable moiety present in the medium (or a sample thereof) is detected. In some embodiments, the resulting signal causes an alteration in a property of the detectable moiety, and such alteration can be detected, e.g., as an optical signal. For example, the signal may alter the emission or absorption of electromagnetic radiation (e.g., radiation having a wavelength within the infrared, visible or UV portion of the spectrum) by the detectable moiety. In some embodiments, a reporter molecule comprises a fluorescent or luminescent moiety, and a second molecule serves as quencher that quenches the fluorescent or luminescent moiety. Such alteration can be detected using apparatus and methods known in the art. In many embodiments of the invention, the reporter molecule is a genetically encodable molecule that can be expressed by a cell, and the detectable moiety comprises, e.g., a detectable polypeptide. Thus in some embodiments, the reporter molecule is a polypeptide comprising a fluorescent polypeptides such as green, blue, sapphire, yellow, red, orange, and cyan fluorescent proteins and derivatives thereof (e.g., enhanced GFP); monomeric red fluorescent protein and derivatives such as those known as “mFruits”, e.g., mCherry, mStrawberry, mTomato, etc., and luminescent proteins such as aequorin. (It will be understood that in some embodiments, the fluorescence or luminescence occurs in the presence of one or more additional molecules, e.g., an ion such as a calcium ion and/or a prosthetic group such as coelenterazine.) In some embodiments, the detectable moiety comprises an enzyme that acts on a substrate to produce a fluorescent, luminescent, colored, or otherwise detectable product. Examples of enzymes that may serve as detectable moieties include luciferase; beta-galactosidase; horseradish peroxidase; alkaline phosphatase; etc. (It will be appreciated that the enzyme is detected by detecting the product of the reaction.) In some embodiments, the detectable moiety comprises a polypeptide tag that can be readily detected using a second agent such as a labeled (e.g., fluorescently labeled) antibody. For example, fluorescently labeled antibodies that bind to the HA, Myc, or a variety of other peptide tags are available. Thus the invention encompasses embodiments in which a detectable moiety can be detected directly (i.e., it generates a detectable signal without requiring interaction with a second agent) and embodiments in which a detectable moiety interacts (e.g., binds and/or reacts) with a second agent and such interaction renders the detectable moiety detectable, e.g., by resulting in generation of a detectable signal or because the second agent is directly detectable. In embodiments in which a detectable moiety interacts with a second agent to produce a detectable signal, the detectable moiety may react with the second agent is acted on by a second agent to produce a detectable signal. In many embodiments, the intensity of the signal provides an indication of the amount of detectable moiety present. e.g., in a sample being asssessed or in area being imaged. In some embodiments, the amount of detectable moiety is optionally quantified, e.g., on a relative or absolute basis, based on the signal intensity.

The description provides nucleic acids comprising a sequence that encodes a reporter polypeptide of the invention. In some embodiments, a nucleic acid encodes a precursor polypeptide of a reporter polypeptide of the invention. In some embodiments, the sequence encoding the polypeptide is operably linked to expression control elements (e.g., a promoter or promoter/enhancer sequence) appropriate to direct transcription of mRNA encoding the polypeptide. The invention further provides expression vectors comprising the nucleic acids.

Selection of appropriate expression control elements may be based, e.g., on the cell type and species in which the nucleic acid is to be expressed. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormones receptor ligands (e.g., steroid receptor ligands, which may or may not be steroids), metal-regulated systems (e.g., metallothionein promoter), etc.

The description further provides cells and cell lines that comprise such nucleic acids and/or vectors. In some embodiments, the cells are eukaryotic cells, e.g., fungal, plant, or animal cells. In some embodiments, the cell is a vertebrate cell, e.g., a mammalian cell, e.g., a human cell, non-human primate cell, or rodent cell. Often a cell is a member of a cell line, e.g., an established or immortalised cell line that has acquired the ability to proliferate indefinitely in culture (e.g., as a result of mutation or genetic manipulation such as the constitutive expression of the catalytic component of telomerase). Numerous cell lines are known in the art and can be used in the instant invention. Mammalian cell lines include, e.g., HEK-293 (e.g., HEK-293T), CHO, NIH-3T3, COS, and HeLa cell lines. In some embodiments, a cell line is a tumor cell line. In other embodiments, a cell is non-tumorigenic and/or is not derived from a tumor. In some embodiments, the cells are adherent cells. In some embodiments, non-adherent cells are used. In some embodiments, a cell is of a cell type or cell line is used that has been shown to naturally have a subset of TR activator genes expressed or TR inhibitor genes not expressed. If a cell lacks one or more TR activator or inhibitor genes, the cell can be genetically engineered to express such protein(s). In some embodiments, a cell line of the invention is descended from a single cell. For example, a population of cells can be transfected with a nucleic acid encoding the reporter polypeptide and a colony derived from a single cell can be selected and expanded in culture. In some embodiments, cells are transiently transfected with an expression vector that encodes the reporter molecule. Cells can be co-transfected with a control plasmid, optionally expressing a different detectable polypeptide, to control for transfection efficiency (e.g., across multiple runs of an assay).

TR Activator and TR Inhibitor Polypeptides and Nucleic Acids TR activator and TR inhibitor genes are listed in FIG. 10. Under the headings “Embryonic Markers” and “Fetal/Adult Markers”, respectively. TR activator and TR inhibitor polypeptides useful in the inventive methods may be obtained by a variety of methods. In some embodiments, the polypeptides are produced using recombinant DNA techniques. Standard methods for recombinant protein expression can be used. A nucleic acid encoding a TR activator or TR inhibitor gene can readily be obtained, e.g., from cells that express the genes (e.g., by PCR or other amplification methods or by cloning) or by chemical synthesis or in vitro transcription based on the cDNA sequence polypeptide sequence. One of ordinary skill in the art would know that due to the degeneracy of the genetic code, the genes can be encoded by many different nucleic acid sequences. Optionally, a sequence is codon-optimized for expression in a host cell of choice. The genes could be expressed in bacterial, fungal, animal, or plant cells or organisms. The genes could be isolated from cells that naturally express it or from cells into which a nucleic acid encoding the protein has been transiently or stably introduced, e.g., cells that contain an expression vector encoding the genes. In some embodiments, the gene is secreted by cells in culture and isolated from the culture medium.

In some embodiments of the invention, the sequence of a TR activator or TR inhibitor polypeptide is used in the inventive screening methods. A naturally occurring TR activator or TR inhibitor polypeptide can be from any species whose genome encodes a TR activator or TR inhibitor polypeptide, e.g., human, non-human primate, rodent, etc. A polypeptide whose sequence is identical to naturally occurring TR activator or TR inhibitor is sometimes referred to herein as “native TR activator/inhibitor”. A TR activator or TR inhibitor polypeptide of use in the invention may or may not comprise a secretion signal sequence or a portion thereof. For example, mature TR activator or TR inhibitor comprising or consisting of amino acids 20-496 of human TR activator or TR inhibitor (or corresponding amino acids of TR activator or TR inhibitor of a different species) may be used.

In some embodiments, a polypeptide comprising or consisting of a variant or fragment of TR activator or TR inhibitor is used. TR activator or TR inhibitor variants include polypeptides that differ by one or more amino acid substitutions, additions, or deletions, relative to TR activator or TR inhibitor. In some embodiments, a TR activator or TR inhibitor variant comprises a polypeptide at least 70%, 75%, 80%, 85%, 90%, 95%, 96%, 97%, 98%, 99%, or more identical to at least amino acids 20-496 of TR activator or TR inhibitor (e.g., from human or mouse) over at least 50%, 60%, 70%, 80%, 85%, 90%, 95%, 96%, 97%, 98%, 99%, or 100% of at least amino acids 20-496 of human TR activator or TR inhibitor or amino acids 20-503 of mouse TR activator or TR inhibitor. In some embodiments, a TR activator or TR inhibitor variant comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. In some embodiments, a TR activator or TR inhibitor polypeptide comprises a polypeptide at least 80%, 85%, 90%, 95%, 96%, 97%, 98%, 99%, or more identical to at least amino acids 20-496 of human TR activator or TR inhibitor or amino acids 20-503 of mouse TR activator or TR inhibitor. A nucleic acid that encodes a TR activator or TR inhibitor variant or fragment can readily be generated, e.g., by modifying the DNA that encodes native TR activator or TR inhibitor using, e.g., site-directed mutagenesis, or by other standard methods, and used to produce the TR activator or TR inhibitor variant or fragment. For example, a fusion protein can be produced by cloning sequences that encode TR activator or TR inhibitor into a vector that provides the sequence encoding the heterologous portion. In some embodiments a tagged TR activator or TR inhibitor is used. For example, in some embodiments a TR activator or TR inhibitor polypeptide comprising a 6xHis tag, e.g., at its C terminus, is used.

### Test Compounds

A wide variety of test compounds can be used in the inventive methods for identifying iTR factors and global modulators of iTR. For example, a test compound can be a small molecule, polypeptide, peptide, nucleic acid, oligonucleotide, lipid, carbohydrate, antibody, or hybrid molecule including but not limited to those described herein, including mRNA for the genes OCT4, SOX2, KLF4, NANOG, ESRRB, NR5A2, CEBPA, MYC, LIN28A and LIN28B alone and in diverse combinations, and in diverse combinations with small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDAC10),III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDAC11) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′eoxycytidine (trade name Vidaza and Azadine); vitamin C which can inhibit DNA methylation, increase Tet1 which increases 5 hmC which is a first step of demethylation; activators of 3′ phosphoinositide-dependent kinase 1 including but not limited to PS48; promoters of glycolysis including but not limited to Quercetin and fructose 2,6-bisphosphate (an activator of phosphofructokinase 1); agents that promote the activity of the HIF1 transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expresson of PCDHB2 or AMH or other activators or iTR as described herein, or in injured or diseased tissues in vivo, or in modulating the lifespan of animals in vivo.

In vitro assays for iTR patterns of expression of the genes COX7A1, PLPP7, and NAALADL1 as well as gene expression or protein markers of pluripotency including DNMT3B, and HELLS or Tra-1-60, Tra-1-81, and SSEA4 respectively are performed to optimize global patterns of iTR gene expression without reverting the target cells to pluripotency. Examples of individual agents and combinations of agents screened are: OCT4, SOX2, KLF4, MYC and LIN28A; OCT4; KLF4; OCT4, KLF4; OCT4, KLF4, LIN28A; OCT4, KLF4, LIN28B; SOX2; MYC; NANOG; ESRRB; NT5A2; OCT4, SOX2, KLF4, and LIN28A; OCT4, SOX2, KLF4, and LIN28B; OCT4, KLF4, MYC and LIN28A; and each of the preceeding combinations of agents together with 0.25 mM NaB, 5 μM PS48 and 0.5 μM A-83-01 during the first four weeks, followed by treatment with 0.25 mM sodium butyrate, 5 μM PS48, 0.5 μM A-83-01 and 0.5 μM PD0325901 each of which is assayed at 0, 1, 2, 4, 7, 10, and 14 days for markers of global modulation of iTR gene expression.

Compounds can be obtained from natural sources or produced synthetically. Compounds can be at least partially pure or may be present in extracts or other types of mixtures whose components are at least in part unknown or uncharacterized. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multwell plates (e.g., 384 well plates, 1596 well plates, etc.). They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, antibody libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability.

In some embodiments, a test compound is substantially non-toxic to cells of an organism to which the compound may be administered and/or to cells with which the compound may be tested, at the concentration to be used or, in some embodiments, at concentrations up to 10-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays. For example, a cellular metabolism assay such as AlamarBlue, MTT, MTS, XTT, and CellTitre Glo assays, a cell membrane integrity assay, a cellular ATP-based viability assay, a mitochondrial reductase activity assay, a BrdU, EdU, or H3-Thymidine incorporation assay could be used. In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

### Assays for Global Modulators of iTR: Aspects of Assay Implementation and Controls

Various inventive screening assays described above involve determining whether a test compound inhibits the levels of active TR inhibitors or increases the levels of active TR activators. Suitable cells for expression of a reporter molecule are described above.

In performing an inventive assay, assay components (e.g., cells, TR activator or TR inhibitor polypeptide, and test compounds) are typically dispensed into multiple vessels or other containers. Any type of vessel or article capable of containing cells can be used. In many embodiments of the invention, the vessels are wells of a multi-well plate (also called a “microwell plate”, “microtiter plate”, etc. For purposes of description, the term “well” will be used to refer to any type of vessel or article that can be used to perform an inventive screen, e.g., any vessel or article that can contain the assay components. It should be understood that the invention is not limited to use of wells or to use of multi-well plates. In some embodiments, any article of manufacture in which multiple physically separated cavities (or other confining features) are present in or on a substrate can be used. For example, assay components can be confined in fluid droplets, which may optionally be arrayed on a surface and, optionally, separated by a water-resistant substance that confines the droplets to discrete locations, in channels of a microfluidic device, etc.

In general, assay components can be added to wells in any order. For example, cells can be added first and maintained in culture for a selected time period (e.g., between 6 and 48 hours) prior to addition of a test compound and target TR activator or TR inhibitor polypeptides or cells with express constructs to a well. In some embodiments, compounds are added to wells prior to addition of polypeptides of cells. In some embodiments, expression of a reporter polypeptide is induced after plating the cells, optionally after addition of a test compound to a well. In some embodiments, expression of the reporter molecule is achieved by transfecting the cells with an expression vector that encodes the reporter polypeptide. In some embodiments, the cells have previously been genetically engineered to express the reporter polypeptide. In some embodiments, expression of the reporter molecule is under control of regulatable expression control elements, and induction of expression of the reporter molecule is achieved by contacting the cells with an agent that induces (or derepresses) expression.

The assay composition comprising cells, test compound, or polypeptide is maintained for a suitable time period during which test compound may (in the absence of a test compound that inhibits its activity) cause an increase or decrease of the level or activity of the target TR activator or TR inhibitor. The number of cells, amount of TR activator or TR inhibitor polypeptide, and amount of test compound to be added will depend, e.g., on factors such as the size of the vessel, cell type, and can be determined by one of ordinary skill in the art. In some embodiments, the ratio of the molar concentration of TR activator or TR inhibitor polypeptide to test compound is between 1:10 and 10:1. In some embodiments, the number of cells, amount of test compound, and length of time for which the composition is maintained can be selected so that a readily detectable level signal after a selected time period in the absence of a test compound. In some embodiments, cells are at a confluence of about 25%-75%, e.g., about 50%, at the time of addition of compounds. In some embodiments, between 1,000 and 10,000 cells/well (e.g., about 5,000 cells/well) are plated in about 100 μl medium per well in 96-well plates. In other exemplary embodiments, cells are seeded in about 30 μl-50 μl of medium at between 500 and 2,000 (e.g., about 1000) cells per well into 384-well plates. In some embodiments, compounds are tested at multiple concentrations (e.g., 2-10 different concentrations) and/or in multiple replicates (e.g., 2-10 replicates). Multiple replicates of some or all different concentrations can be performed. In some embodiments, candidate TR factors are used at a concentration between 0.1 μg/ml and 100 μg/ml, e.g., 1 μg/ml and 10 μg/ml. In some embodiments, candidate TR factors are used at multiple concentrations. In some embodiments, compounds are added to cells between 6 hours and one day (24 hr) after seeding.

In some aspects of any of the inventive compound screening and/or characterization methods, a test compound is added to an assay composition in an amount sufficient to achieve a predetermined concentration. In some embodiments the concentration is up to about 1 nM. In some embodiments the concentration is between about 1 nM and about 100 nM. In some embodiments the concentration is between about 100 nM and about 10 μM. In some embodiments the concentration is at least 10 μM, e.g., between 10 μM and 100 μM. The assay composition can be maintained for various periods of time following addition of the last component thereof. In certain embodiments the assay composition is maintained for between about 10 minutes and about 4 days, e.g., between 1 hour and 3 days, e.g., between 2 hours and 2 days, or any intervening range or particular value, e.g., about 4-8 hours, after addition of all components. Multiple different time points can be tested. Additional aliquots of test compound can be added to the assay composition within such time period. In some embodiments, cells are maintained in cell culture medium appropriate for culturing cells of that type. In some embodiments, a serum-free medium is used. In some embodiments, the assay composition comprises a physiologically acceptable liquid that is compatible with maintaining integrity of the cell membrane and, optionally, cell viability, instead of cell culture medium. Any suitable liquid could be used provided it has the proper osmolarity and is otherwise compatible with maintaining reasonable integrity of the cell membrane and, optionally, cell viability, for at least a sufficient period of time to perform an assay. One or more measurements indicative of an increase in the level of active TR activator or decrease in TR inhibitor can be made during or following the incubation period.

In some embodiments, the compounds screened for potential to be global modulators of iTR are chosen from agents capable in other conditions of inducing pluripotency in somatic cell types. Such agents include the following compounds individually or in combination: the genes OCT4, SOX2, KLF4, NANOG, ESRRB, NR5A2, CEBPA, MYC, LIN28A and LIN28B alone and in combination with small molecule compounds such as combinations of the following compounds: inhibitors of glycogen synthase 3 (GSK3) including but not limited to CHIR99021; inhibitors of TGF-beta signaling including but not limited to 5B431542, A-83-01, and E616452; HDAC inhibitors including but not limited to aliphatic acid compounds including but not limited to:

valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDAC10), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDAC11) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycytidine (trade name Vidaza and Azadine); vitamin C which can inhibit DNA methylation, increase Tet1 which increases 5 hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1);

agents that promote the activity of the HIF 1 transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expresson of PCDHB2 or AMH or other activators or iTR as described herein, or in injured or diseased tissues in vivo, or in modulating the lifespan of animals in vivo.

In some embodiments, individual compounds, each typically of known identity (e.g., structure and/or sequence), are added to each of a multiplicity of wells. In some embodiments, two or more compounds may be added to one or more wells. In some embodiments, one or more compounds of unknown identity may be tested. The identity may be determined subsequently using methods known in the art.

In various embodiments, foregoing assay methods of the invention are amenable to high-throughput screening (HTS) implementations. In some embodiments, the screening assays of the invention are high throughput or ultra high throughput (see, e.g., Fernandes, P. B., Curr Opin Chem. Biol. 1998, 2:597; Sundberg, S A, Curr Opin Biotechnol. 2000, 11:47). High throughput screens (HTS) often involve testing large numbers of compounds with high efficiency, e.g., in parallel. For example, tens or hundreds of thousands of compounds can be routinely screened in short periods of time, e.g, hours to days. In some embodiments, HTS refers to testing of between 1,000 and 100,000 compounds per day. In some embodiments, ultra high throughput refers to screening in excess of 100,000 compounds per day, e.g., up to 1 million or more compounds per day. The screening assays of the invention may be carried out in a multi-well format, for example, a 96-well, 384-well format, 1,536-well format, or 3,456-well format and are suitable for automation. In some embodiments, each well of a microwell plate can be used to run a separate assay against a different test compound or, if concentration or incubation time effects are to be observed, a plurality of wells can contain test samples of a single compound, with at least some wells optionally being left empty or used as controls or replicates. Typically, HTS implementations of the assays disclosed herein involve the use of automation. In some embodiments, an integrated robot system including one or more robots transports assay microwell plates between multiple assay stations for compound, cell and/or reagent addition, mixing, incubation, and readout or detection. In some aspects, an HTS system of the invention may prepare, incubate, and analyze many plates simultaneously. Suitable data processing and control software may be employed. High throughput screening implementations are well known in the art. Without limiting the invention in any way, certain general principles and techniques that may be applied in embodiments of a HTS of the present invention are described in Macarron R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006). An additional compound may, for example, have one or more improved pharmacokinetic and/or pharmacodynamic properties as compared with an initial hit or may simply have a different structure. An “improved property” may, for example, render a compound more effective or more suitable for one or more purposes described herein. In some embodiments, for example, a compound may have higher affinity for the molecular target of interest (e.g., TR activator or TR inhibitor gene products), lower affinity for a non-target molecule, greater solubility (e.g., increased aqueous solubility), increased stability (e.g., in blood, plasma, and/or in the gastrointestinal tract), increased half-life in the body, increased bioavailability, and/or reduced side effect(s), etc. Optimization can be accomplished through empirical modification of the hit structure (e.g., synthesizing compounds with related structures and testing them in cell-free or cell-based assays or in non-human animals) and/or using computational approaches. Such modification can in some embodiments make use of established principles of medicinal chemistry to predictably alter one or more properties. In some embodiments, one or more compounds that are “hit” are identified and subjected to systematic structural alteration to create a second library of compounds (e.g., refined lead compounds) structurally related to the hit. The second library can then be screened using any of the methods described herein. In some embodiments, an iTR factor is modified or incorporates a moiety that enhances cstability (e.g., in serum), increases half-life, reduces toxicity or immunogenicity, or otherwise confers a desirable property on the compound.

Uses of iTR, iTM, and ICM Factors

### Pharmaceutical Compositions

iTR, iTM, and iCM factors have a variety of different uses. Non-limiting examples of such uses are discussed herein. In some embodiments, an iTR factor is used to enhance regeneration of an organ or tissue. In some embodiments, an iTR factor is used to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, an iTR factor is used to enhance regeneration of a stromal layer, e.g., a connective tissue supporting the parenchyma of a tissue. In some embodiments, an iTR factor is used to enhance regeneration following surgery, e.g., surgery that entails removal of at least a portion of a diseased or damaged tissue, organ, or other structure such as a limb, digit, etc. For example, such surgery might remove at least a portion of a liver, lung, kidney, stomach, pancreas, intestine, mammary gland, ovary, testis, bone, limb, digit, muscle, skin, etc. In some embodiments, the surgery is to remove a tumor. In some embodiments, an iTR factor is used to promote scarless regeneration of skin following trauma, surgery, disease, and burns.

Enhancing regeneration can include any one or more of the following, in various embodiments: (a) increasing the rate of regeneration; (b) increasing the extent of regeneration; (c) promoting establishment of appropriate structure (e.g., shape, pattern, tissue architecture, tissue polarity) in a regenerating tissue or organ or other body structure; (d) promoting growth of new tissue in a manner that retains and/or restores function. While use of an iTR factor to enhance regeneration is of particular interest, the invention encompasses use of an iTR factor to enhance repair or wound healing in general, without necessarily producing a detectable enhancement of regeneration. Thus, the invention provides methods of enhancing repair or wound healing, wherein an iTR factor is administered to a subject in need thereof according to any of the methods described herein.

In some embodiments, the invention provides a method of enhancing regeneration in a subject in need thereof, the method comprising administering an effective amount of an iTR factor to the subject. In some embodiments, an effective amount of a compound (e.g., an iTR factor) is an amount that results in an increased rate or extent of regeneration of damaged tissue as compared with a reference value (e.g., a suitable control value). In some embodiments, the reference value is the expected (e.g., average or typical) rate or extent of regeneration in the absence of the compound (optionally with administration of a placebo). In some embodiments, an effective amount of an iTR factor is an amount that results in an improved structural and/or functional outcome as compared with the expected (e.g., average or typical) structural or functional outcome in the absence of the compound. In some embodiments, an effective amount of a compound, e.g., an iTR factor, results in enhanced blastema formation and/or reduced scarring. Extent or rate of regeneration can be assessed based on dimension(s) or volume of regenerated tissue, for example. Structural and/or functional outcome can be assessed based on, e.g., visual examination (optionally including use of microscopy or imaging techniques such as X-rays, CT scans, MRI scans, PET scans) and/or by evaluating the ability of the tissue, organ, or other body part to perform one or more physiological processes or task(s) normally performed by such tissue, organ, or body part. Typically, an improved structural outcome is one that more closely resembles normal structure (e.g., structure that existed prior to tissue damage or structure as it exists in a normal, healthy individual) as compared with the structural outcome that would be expected (e.g., average or typical outcome) in the absence of treatment with an iTR factor. One of ordinary skill in the art can select an appropriate assay or test for function. In some mbodiments, an increase in the rate or extent of regeneration as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile). It will be appreciated that in many embodiments an iTR modulator, e.g., an iTR factor, administered to a subject of a particular species (e.g., for therapeutic purposes) is a compound that modulates, e.g., inhibits, the endogenous TR genes expressed in subjects of that species. For example, if a subject is human, a compound that inhibits the activity of human TR inhibitor gene products and activates the activity of human TR activator gene products would typically be administered.

In some embodiments, the iTR factor is used to enhance skin regeneration, e.g., after a burn (thermal or chemical), scrape injury, or other situations involving skin loss, e.g., infections such as necrotizing fasciitis or purpura fulminans. In some embodiments, a burn is a second or third degree burn. In some embodiments a region of skin loss has an area of at least 10 cm². In one aspect, an iTR factor enhances regeneration of grafted skin. In one aspect, an iTR factor reduces excessive and/or pathological wound contraction or scarring.

In some embodiments, an iTR factor is used to enhance bone regeneration, e.g., in a situation such as non-union fracture, implant fixation, periodontal or alveolar ridge augmentation, craniofacial surgery, or other conditions in which generation of new bone is considered appropriate. In some embodiments, an iTR factor is applied to a site where bone regeneration is desired. In some embodiments, an iTR factor is incorporated into or used in combination with a bone graft material. Bone graft materials include a variety of ceramic and proteinaceous materials. Bone graft materials include autologous bone (e.g., bone harvested from the iliac crest, fibula, ribs, etc.), allogeneic bone from cadavers, and xenogeneic bone. Synthetic bone graft materials include a variety of ceramics such as calcium phosphates (e.g. hydroxyapatite and tricalcium phosphate), bioglass, and calcium sulphate, and proteinaceous materials such as demineralized bone matrix (DBM). DBM can be prepared by grinding cortical bone tissues (generally to 100-500 μm sieved particle size), then treating the ground tissues with hydrochloric acid (generally 0.5 to 1 N). In some embodiments, an iTR factor is administered to a subject together with one or more bone graft materials. The iTR factor may be combined with the bone graft material (in a composition comprising an iTR factor and a bone graft material) or administered separately, e.g., after placement of the graft. In some embodiments, the invention provides a bone paste comprising an iTR factor. Bone pastes are products that have a suitable consistency and composition such that they can be introduced into bone defects, such as voids, gaps, cavities, cracks etc., and used to patch or fill such defects, or applied to existing bony structures. Bone pastes typically have sufficient malleability to permit them to be manipulated and molded by the user into various shapes. The desired outcome of such treatments is that bone formation will occur to replace the paste, e.g., retaining the shape in which the paste was applied. The bone paste provides a supporting structure for new bone formation and may contain substance(s) that promote bone formation. Bone pastes often contain one or more components that impart a paste or putty-like consistency to the material, e.g., hyaluronic acid, chitosan, starch components such as amylopectin, in addition to one or more of the ceramic or proteinaceous bone graft materials (e.g., DBM, hydroxyapatite) mentioned above.

In some embodiments, an iTR factor enhances the formation and/or recruitment of osteoprogenitor cells from undifferentiated mesechymal cells and/or enhances the differentiation of osteoprogenitor cells into cells that form new bone (osteoblasts).

In some embodiments, an iTR factor is administered to a subject with osteopenia or osteoporosis, e.g., to enhance bone regeneration in the subject.

In some embodiments, an iTR factor is used to enhance regeneration of a joint (e.g., a fibrous, cartilaginous, or synovial joint). In some embodiments, the joint is an intervertebral disc. In some embodiments, a joint is a hip, knee, elbow, or shoulder joint. In some embodiments, an iTR factor is used to enhance regeneration of dental and/or periodontal tissues or structures (e.g., pulp, periodontal ligament, teeth, periodontal bone).

In some embodiments, an iTR factor is used to reduce glial scarring in CNS and PNS injuries.

In some embodiments, an iTR factor is used to reduce adhesions and stricture formation in internal surgery.

In some embodiments, an iTR factor is used to decrease scarring in tendon and ligament repair improving mobility.

In some embodiments, an iTR factor is used to reduce vision loss following eye injury.

In some embodiments, an iTR factor is administered to a subject in combination with cells. The iTR factor and the cells may be administered separately or in the same composition. If administered separately, they may be administered at the same or different locations. The cells can be autologous, allogeneic, or xenogeneic in various embodiments. The cells can comprise progenitor cells or stem cells, e.g., adult stem cells. As used herein, a stem cell is a cell that possesses at least the following properties: (i) self-renewal, i.e., the ability to go through numerous cycles of cell division while still maintaining an undifferentiated state; and (ii) multipotency or multidifferentiative potential, i.e., the ability to generate progeny of several distinct cell types (e.g., many, most, or all of the distinct cell types of a particular tissue or organ). An adult stem cell is a stem cell originating from non-embryonic tissues (e.g., fetal, post-natal, or adult tissues). As used herein, the term “progenitor cell” encompasses cells multipotencand cells that are more differentiated than pluripotent stem cells but not fully differentiated. Such more differentiated cells (which may arise from embryonic progenitor cells) have reduced capacity for self-renewal as compared with embryonic progenitor cells. In some embodiments, an iTR factor is administered in combination with mesenchymal progenitor cells, neural progenitor cells, endothelial progenitor cells, hair follicle progenitor cells, neural crest progenitor cells, mammary stem cells, lung progenitor cells (e.g., bronchioalveolar stem cells), muscle progenitor cells (e.g., satellite cells), adipose-derived progenitor cells, epithelial progenitor cells (e.g., keratinocyte stem cells), and/or hematopoietic progenitor cells (e.g., hematopoietic stem cells). In some embodiments, the cells comprise induced pluripotent stem cells (iPS cells), or cells that have been at least partly differentiated from iPS cells. In some embodiments, the progenitor cells comprise adult stem cells. In some embodiments, at least some of the cells are differentiated cells, e.g., chondrocytes, osteoblasts, keratinocytes, hepatocytes. In some embodiments, the cells comprise myoblasts.

In some embodiments, an iTR factor is administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, cross-linking agents, etc. The composition may be applied (e.g., using a syringe) to an area where regeneration is needed, where it forms a gel in situ, from which an iTR factor is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8):1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, an iTR factor is administered to a subject in combination with vectors expressing the catalytic component of telomerase. The vector may be administered separately or in the same composition. If administered separately, they may be administered at the same or different locations. The vector may express the telomerase catalytic component from the same species as the treated tissue or from another species. Said co-administration of the iTR factor with the telomerase catalytic component is particularly useful wherein the target tissue is from an aged individual and said individual is from the human species.

Other inventive methods comprise use of an iTR factor in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a functional tissue or organ can be promoted by contacting an iTR factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTR factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM). Optionally, an iTR factor is administered to the subject prior to, during, and/or following grafting of the ex vivo generated tissue or organ. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with an iTR factor or combinations of factors including those capable of causing a global pattern of iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, an iTR factor or combination of factors is administered directly to or near a site of tissue damage “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, an iTR factor is applied to the remaining portion of the tissue, organ, or other structure. In some embodiments, an iTR factor is applied to the end of a severed digit or limb) that remains attached to the body, to enhance regeneration of the portion that has been lost. In some embodiments, the severed portion is reattached surgically, and an iTR factor is applied to either or both faces of the wound. In some embodiments, an iTR factor is administered to enhance engraftment or healing or regeneration of a transplanted organ or portion thereof. In some embodiments, an iTR factor is used to enhance nerve regeneration. For example, an iTR factor may be infused into a severed nerve, e.g., near the proximal and/or distal stump. In some embodiments, an iTR factor is placed within an artificial nerve conduit, a tube composed of biological or synthetic materials within which the nerve ends and intervening gap are enclosed. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments the iTR factor is anti-Mullerian hormone (AMH) which may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or in vivo at a concentrations ranging from 0.05-5 mM valproic acid, preferably 1-100 ng/mL, preferably 10 ng/mL.

In some embodiments the iTR factor is GFER (Augmenter of Liver Regeneration (ALR)) in either the shorter secreted form or the longer form that localizes to the mitochondrial intermembrane space which is expressed in relatively higher levels in embryonic tissue and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissues in vivo at a concentration ranging from 2-200 ng/mL, preferably 20 ng/mL.

In some embodiments the iTR factor is valproic acid and may or may not be formulated for localization and slow release in carboxymethyl hyaluronate crosslinked by PEGDA with carboxymethyl-modified gelatin (HyStem-C) to induce iTR, typically at a concentration sufficient to expose cells in vitro or cells in tissue in vivo at a concentration ranging from 0.05-5 mM, preferably 0.5 mM.

In some embodiments the iTR factor is any combination of valproic acid at a concentration of 0.05-5mM, preferably 0.5 mM, GFER protein (either the long or short form) at a concentration of 2-200 ng/mL, preferably 20 ng/mL and AMH protein at a concentration of 1-100 ng/mL, preferably 10 ng/mL. Said combination of the factors valproic acid, GFER, and AMH and may or may not be formulated for localization and slow release in carboxymethyl hyaluronate crosslinked by PEGDA with carboxymethyl-modified gelatin (HyStem-C) to induce iTR.

iTM and iCM factors such as exosomes derived from fetal or adult cells can be administered in physiological solutions such as saline, or slow-released in carboxymethyl hyaluronate crosslinked by PEGDA with carboxymethyl-modified gelatin (HyStem-C) to induce iTM or iCM.

In some embodiments, the gene LIN28B normally expressed primarily in the embryonic phases of development is exogenously expressed in blood cell types including CD34+ hematopoietic cells to promote their proliferation and engraftment into bone marrow in vivo comparable to the proliferative and engraftment capacity of their fetal liver-derived counterparts.

In some embodiments, tissue regeneration is augmented through the administration of prolotherapeutic agents including but not limited to hyperosmolar dextrose, glycerine, lidocaine, phenol, local anestheticphenol, and sodium morrhuate; sclerotherapeutic agents including but not limited to those used to treat blood vessel and lymphatic malformations (vascular malformations) including Klippel Trenaunay syndrome, spider veins, smaller varicose veins, hemorrhoids and hydroceles wherein the agents used include such agents as sodium tetradecyl sulfate or polidocanol wherein the sclerosant is injected into the vessels; and platelet rich plasma-derived factors; wherein the prolotherapeutic, sclerotherapeutic or platelet rich plasma-derived factors are formulated in a matrix to localize their effects or facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments, an iTR factor or combinations of factors is used to promote production of hair follicles and/or growth of hair. In some embodiments, an iTR factor triggers regeneration of hair follicles from epithelial cells that do not normally form hair. In some embodiments, an iTR factor is used to treat hair loss, hair sparseness, partial or complete baldness in a male or female. In some embodiments, baldness is the state of having no or essentially no hair or lacking hair where it often grows, such as on the top, back, and/or sides of the head. In some embodiments, hair sparseness is the state of having less hair than normal or average or, in some embodiments, less hair than an individual had in the past or, in some embodiments, less hair than an individual considers desirable. In some embodiments, an iTR factor is used to promote growth of eyebrows or eyelashes. In some embodiments, an iTR factor is used to treat androgenic alopecia or “male pattern baldness” (which can affect males and females). In some embodiments, an iTR factor is used to treat alopecia areata, which involves patchy hair loss on the scalp, alopecia totalis, which involves the loss of all head hair, or alopecia universalis, which involves the loss of all hair from the head and the body. In some embodiments, an iTR factor is applied to a site where hair growth is desired, e.g., the scalp or eyebrow region. In some embodiments, an iTR factor is applied to or near the edge of the eyelid, to promote eyelash growth. In some embodiments, an iTR factor is applied in a liquid formulation. In some embodiments an iTR factor is applied in a cream, ointment, paste, or gel. In some embodiments, an iTR factor is used to enhance hair growth after a burn, surgery, chemotherapy, or other event causing loss of hair or hear-bearing skin.

In some embodiments, an iTR factor or combination of factors are administered to tissues afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said iTR factors are co-administered with a vector expressing the catalytic component of telomerase to extend cell lifespan.

In some embodiments, an iTR factor or factors are administered to enhance replacement of cells that have been lost or damaged due to insults such as chemotherapy, radiation, or toxins. In some embodiments such cells are stromal cells of solid organs and tissues.

Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti).

In some embodiments, a compound or composition is administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments a compound or composition is administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case of regeneration after tissue damage, it may be desirable to remove (e.g., by surgical excision or debridement) at least some of the damaged tissue. In some embodiments, an iTR factor is administered at or near the site of such removal or abrasion.

In some embodiments, an iTR factor is used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances a developmental disorder resulting in hypoplasia of a tissue, organ, or other body structure becomes evident after birth. In some embodiments, an iTR factor is administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering an iTR factor to the subject. In some embodiments, an iTR factor is administered to the subject prior to birth, i.e., in utero. The various aspects and embodiments of the invention described herein with respect to regeneration are applicable to such de novo generation of a tissue, organ, or other body structure and are encompassed within the invention.

In some aspects, an iTR factor is used to enhance generation of tissue in any of a variety of situations in which new tissue growth is useful at locations where such tissue did not previously exist. For example, generating bone tissue between joints is frequently useful in the context of fusion of spinal or other joints.

iTR factors may be tested in a variety of animal models of regeneration. In one aspect, a modulator of iTR is tested in murine species. For example, mice can be wounded (e.g., by incision, amputation, transection, or removal of a tissue fragment). An iTR factor is applied to the site of the wound and/or to a removed tissue fragment and its effect on regeneration is assessed. The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282(48):35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

In some embodiments, LIN28B is exogenously expressed in blood cell types including CD34+ hematopoietic cells to promote their proliferation and engraftment into bone marrow in vivo comparable to the proliferative and engraftment capacity of their fetal liver-derived counterparts.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such comporisition.

For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 mg/kg to about 100 mg/kg body weight, about 0.01 to about 25 mg/kg body weight, about 0.1 to about 20 mg/kg body weight, about 1 to about 10 mg/kg. Other exemplary doses include, for example, about 1 μg/kg to about 500 mg/kg, about 100 μg/kg to about 5 mg/kg. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc.

Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more compounds or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments an iTR factor is administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo. Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### Sources of iTM and iCM Factors

iTM and iCM factors may be identified by exposing embryonic cells lacking markers of the EFT (such as, by way of nonlimiting example, stromal cells not expressing COX7A1) to a variety of agents and assaying for the induction of said markers such as COX7A1 or reporter constructs such as GFP expressed using the COX7A1 gene promoter.

Since exosomes carry potent protein and RNA factors capable of reprogramming cells to confer new growth, migration and differentiation properties, we examined whether they are capable of reprogramming the developmental state of a cell, i.e. iTM and iCM. We therefore tested exosomes from adult cells for induction of adult genes in embryonic cells. We assessed total RNA expression profile using Illumina microarray analysis of a series of 15 hESC derived clonal embryonic progenitor cell lines and compared these to 18 primary endothelial cell lines (newborn to adult) obtained from various anatomical sites (not shown). We determined that exosomes from cells that have passed the EFT are capable of inducing the expression of COX7A1 in embryonic cells previously lacking such expression, as well as maturing the cells using other markers described herein.

### EXAMPLES

Example 1. Training the DNN with online microarray data and data from cultured clonal embryonic Progenitor cell lines.

We used data from public databases Gene Expression Omnibus (GEO) and ArrayExpress, as well as additional dataset provided by BioTime, Inc. Each gathered sample belongs to one of the following broad cell classes: embryonic stem cells (ESC), induced pluripotent stem cells (iPSC), embryonic progenitor cells (EPC), adult stem cells (ASC) and adult cells (AC). Samples in this example were obtained from the following microarray platforms:

Illumina HumanHT-12 V4.0 (GPL10558), Illumina HumanHT-12 V3.0 (GPL6947), Affymetrix HT Human Genome U133A Array (GPL3921), Affymetrix GeneChip Human Genome U133 Array Set HG-U133A (GPL4557), Affymetrix Human Exon 1.0 ST Array (GPL5188), Affymetrix Human Genome U133 Plus 2.0 Array (GPL570), Affymetrix Human Genome U133A 2.0 Array (GPL571), Affymetrix Human Gene 1.0 ST Array (GPL6244), Affymetrix Human Genome U133A Array (GPL96), Affymetrix Human Genome U133 Plus 2.0 Array (GPL11670). When working with processed data, no distinction was made between platforms by same vendor. The choice of cell classes was motivated by the need to get the largest variety of stem cell development stages. Adult cells included cells from following tissues: kidney, liver, muscle, blood, and neural tissue. Adult stem cells included adipose derived stem cells, epithelial stem cells, hematopoietic stem cells, mesenchymal stem cells, and neural stem cells.

We gathered and preprocessed transcriptomic profiles of 12,404 healthy untreated tissue samples from Affymetrix (4,822 samples) and Illumina (7,582 samples) microarray platforms. Collected samples were assigned to five categories: embryonic stem cells (ESCs), induced pluripotent stem cell (iPSCs), embryonic progenitor cells (EPCs), adult stem cells (ASCs) and adult cells (ACs). The populations of collected samples were substantially homogeneous.

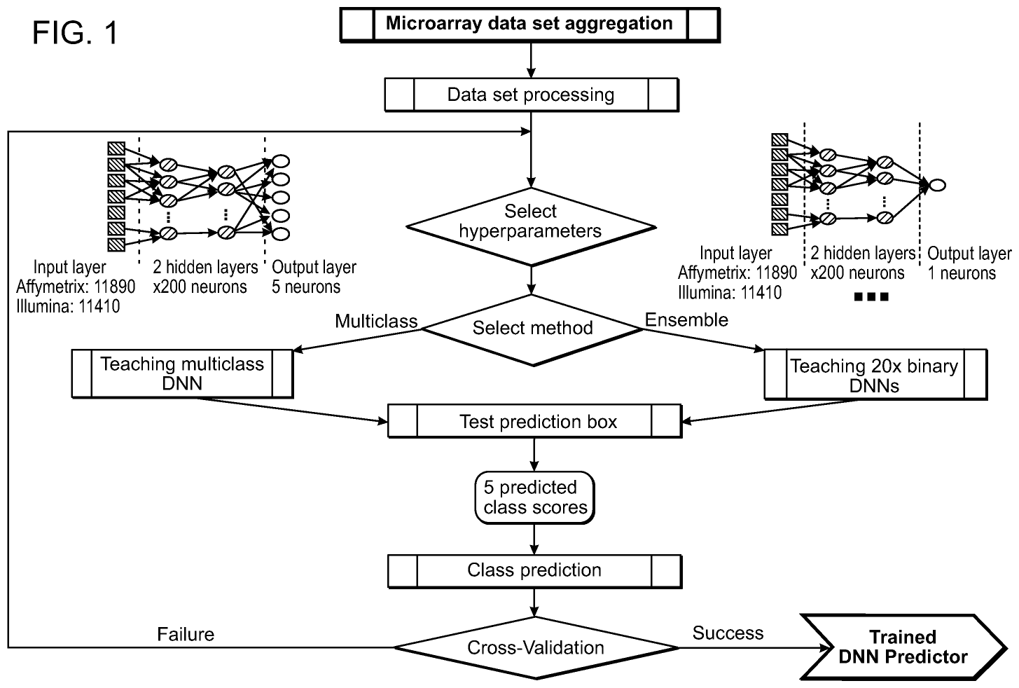

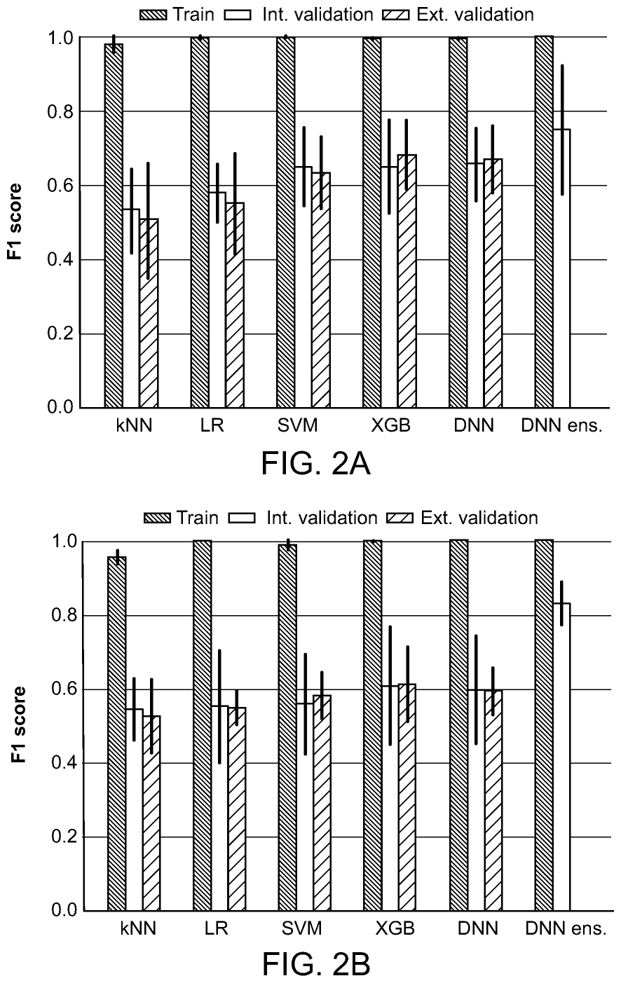

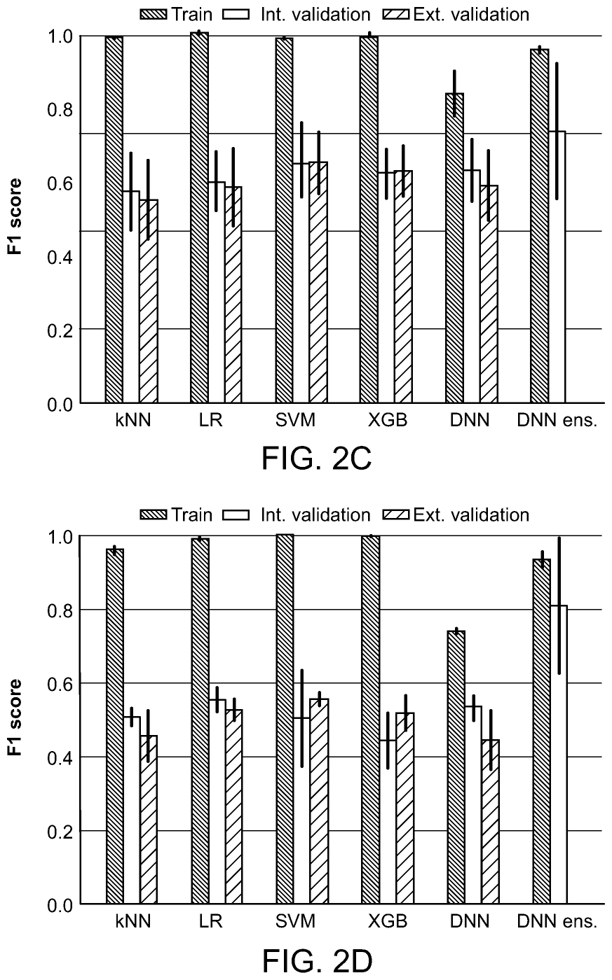

For each microarray platform, we then separately trained six different classifiers: K-nearest neighbors (kNN), logistic regression with PCA-based dimensionality reduction (LR), support vector machines (SVM), gradient boosting machines (GBM), multiclass deep neural network (DNN), and ensemble of 20 deep neural networks (DNN ens.). For all classifiers except DNN ensemble we performed hyperparameter search, while for DNN ensemble we used optimal network hyperparameters obtained for single DNN. Since using single multiclass DNN proved to have a number of drawbacks, we also developed a more computationally demanding but more accurate method, which employs an ensemble of two-class deep neural networks (FIG. 1). We trained 20 binary networks for each target platform set (Affymetrix and Illumina) to perform pairwise (one-vs-one) classification. Then we evaluated the overall ensemble vote for each class as the sum of four one-vs-one networks, which perform pairwise distinction of this class from four other classes. This minimized false-positive votes for any of the classes and achieved a smoother distribution of embryonic scores. Classification performances are shown in FIG. 2.

On Affymetrix microarray gene level data, deep neural networks ensemble achieved mean 0.99 F1 score on train dataset, and 0.75 on validation dataset, while other methods managed to achieve 0.50-0.64 F1 score on external validation dataset. As for Illumina microarray gene level data, deep neural network achieved mean 0.99 Fl score on training dataset, and 0.83 on external validation dataset, while other methods managed to achieve 0.52-0.58 F1 score on external validation dataset.

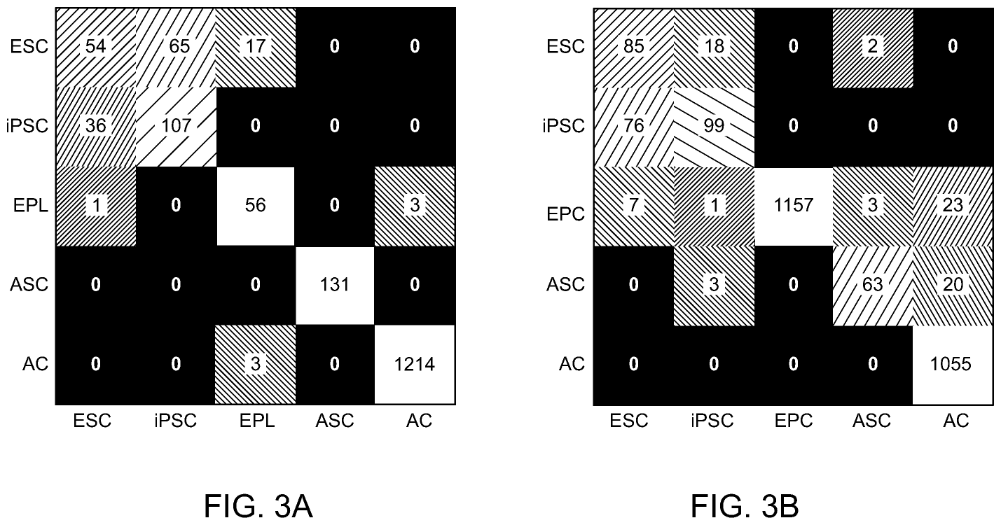

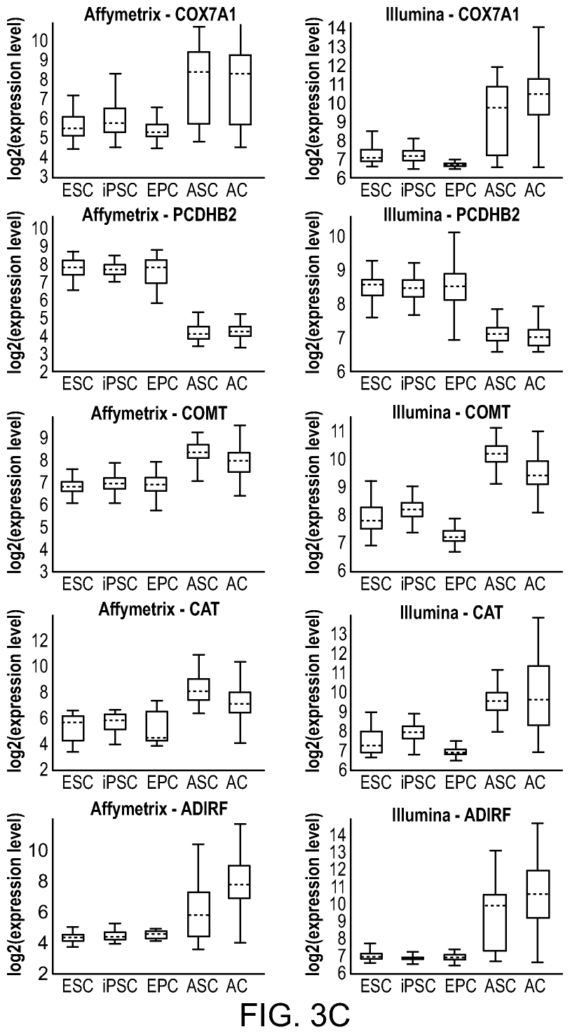

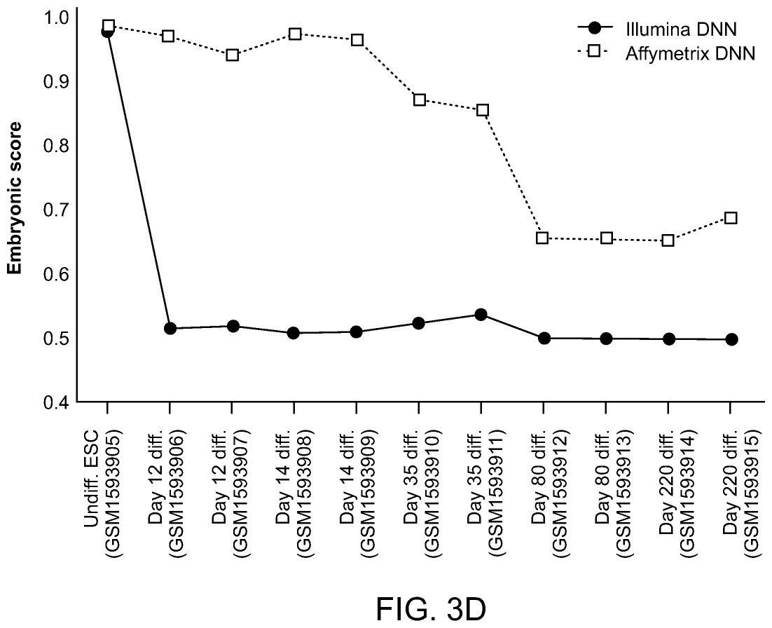

As we can see, classical methods, such as kNN and LR are performing noticeably worse than SVM, XGB and DNN methods. Substantially better performance (about 12% relative improvement for Affymetrix, and 36% relative improvement for Illumina) was achieved using DNN ensemble. For pathway level analysis we used previously established pathway analysis method called OncoFinder described herein. It retains the information about biological function and allows for dimensionality reduction. On the pathway level (FIG. 3c,d) we can see that despite lower accuracy on training set DNN ensemble performance was on validation set is similar to what was achieved at the gene level with F1 scores 0.74 and 0.81 for Affymetrix and Illumina platforms, respectively.

We would like to notice the importance of using labeled cross validation procedure. If samples from same dataset are present in both the training and validation sets, the classifier performance is greatly overestimated (validation performance above 0.9 for all methods; data not shown). This emphasizes the degree to which batch effects affect transcriptomic data, and the need for careful selection of cross validation procedure in order to obtain unbiased estimation of classifier behavior on new data sets.

Based on the output of DNN ensemble we developed an integrative Embryonic Score (ES). To examine the performance of DNN ensemble based score we used a transcriptomic data set consisting of samples belonging to different stages of neural differentiation. This data set was profiled on Affymetrix platform and we can observe a clear decrease of ES with the differentiation stage (FIG. 3d). For Affymetrix-based DNN ensemble the first significant drop in ES score happens between early (day 14) and mid (day 35) radial glial cells and after that drops again and levels off around ES-0.65 for late radial glial cells (day 80) and long term neural precursor cells (day 220). On the other hand, Illumina-based DNN ensemble was far more sensitive and the ES dropped instantly once the cell differentiated from human ESCs (line H9). As shown in FIG. 3c, the genes COX7A1, PCDHB2, COMT, CAT, and ADIRF (cl0orf116) were differentially expressed in embryonic vs adult cell types.

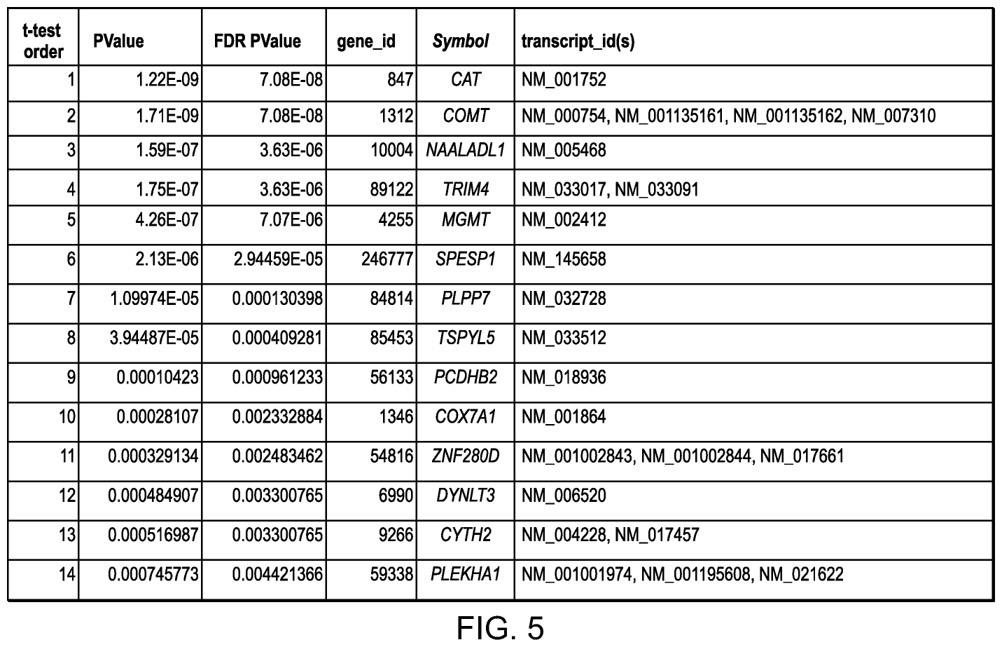

To validate the genes as markers of mammalian EFT, we examined at total of 83 discriminator genes shown in FIG. 4 using RNA-seq in a panel of 15 diverse adult-derived cell types representing derivatives of endoderm, mesoderm, ectoderm, and neural crest cell types as well as 17 clonal embryonic progenitor cell lines. Analysis using t-test showed 14 markers with an FDR P<0.005 (FIG. 5); namely, CAT, COMT, TRIM4, NAALADL1, MGMT, SPESP1, PLPP7, TSPYL5, PCDHB2, COX7A1, ZNF280D, DYNLT3, CYTH2, and PLEKHA1. Of these 14 markers, the 11 genes CAT, COMT, TRIM4, NAALADL1, MGMT, SPESP1, PLPP7, TSPYL5, COX7A1, ZNF280D, and DYNLT3 showed increased expression in adult-derived cells, and the two genes PCDHB2, CYTH2, and PLEKHA1 showed increased expression in the embryonic progenitors. LIN28A, previously identified as a possible regulator of embryonic regenerative potential was not differentially expressed while LIN28B was expressed in a subset of the embryonic progenitor cell lines, particularly those with markers of vascular endothelium, but not in their adult counterparts.

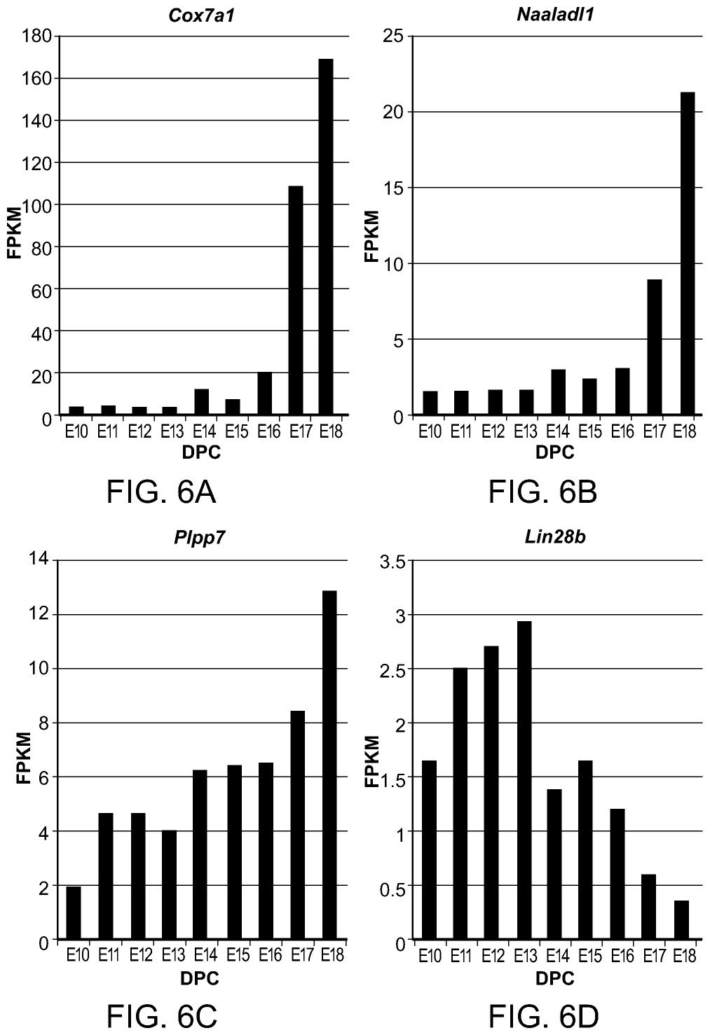

To further validate the 13 genes as markers of the EFT, expression levels were determined in whole mice at embryonic time points spanning the murine EFT (assumed to correspond approximately to Carnegie Stage 23/Theiler Stage 24 or E16. As shown in FIG. 6, the genes COX7A1, NAALADL1, and PLPP7 showed a marked up-regulation at a time point approximating the murine EFT while the expression of the gene LIN28B decreased during the same time period.

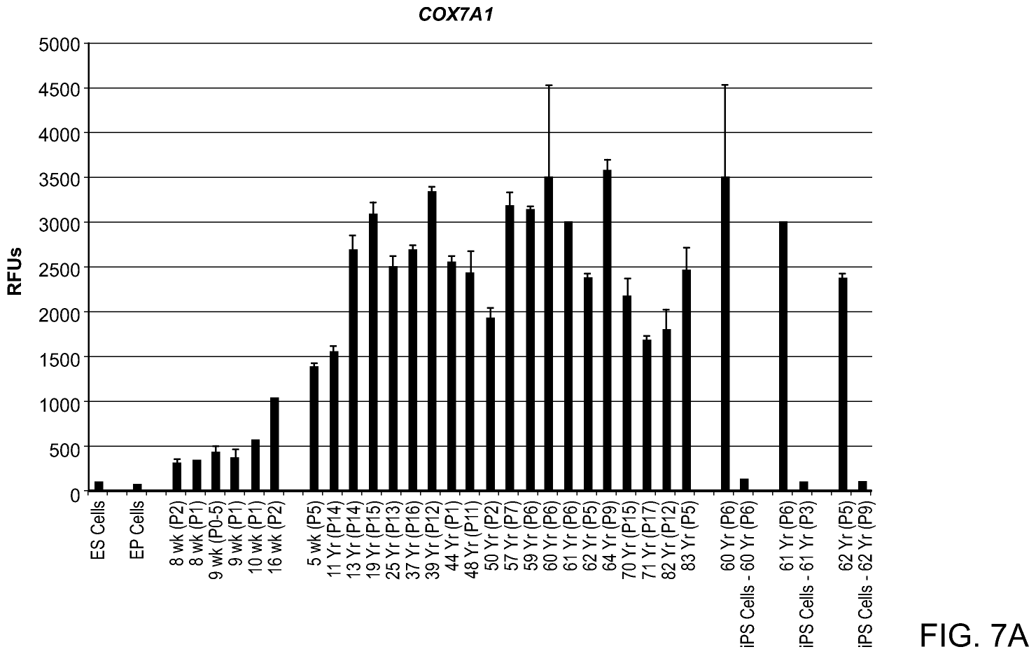

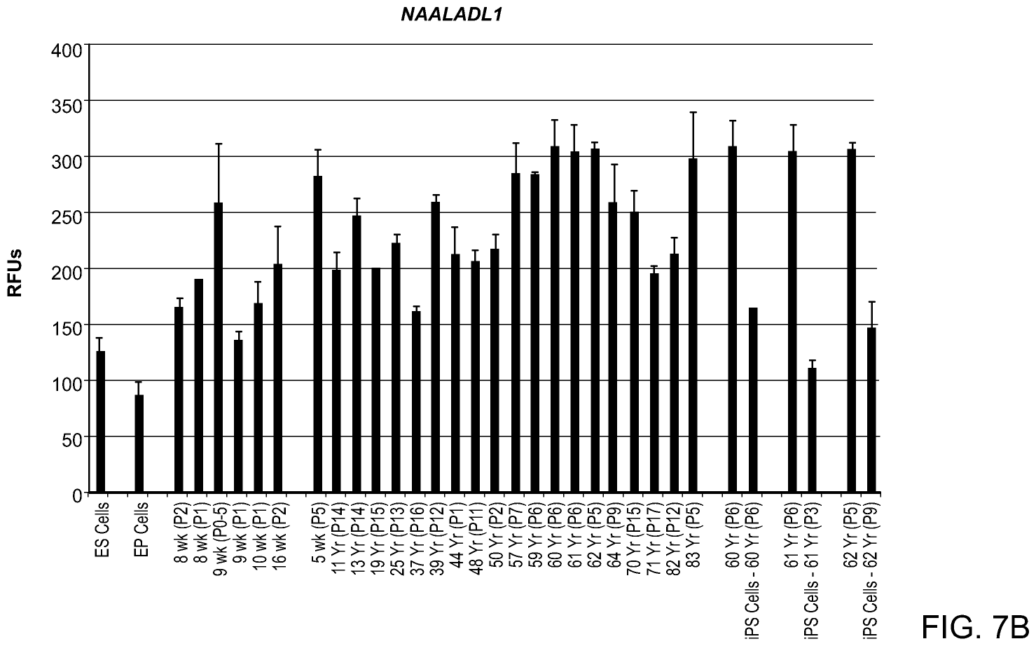

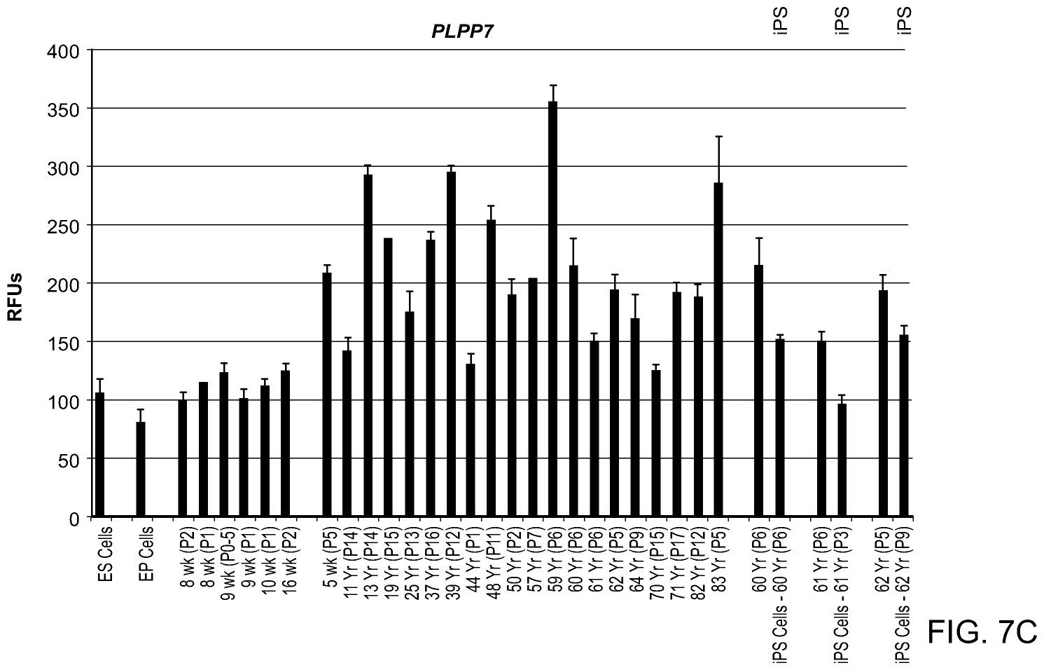

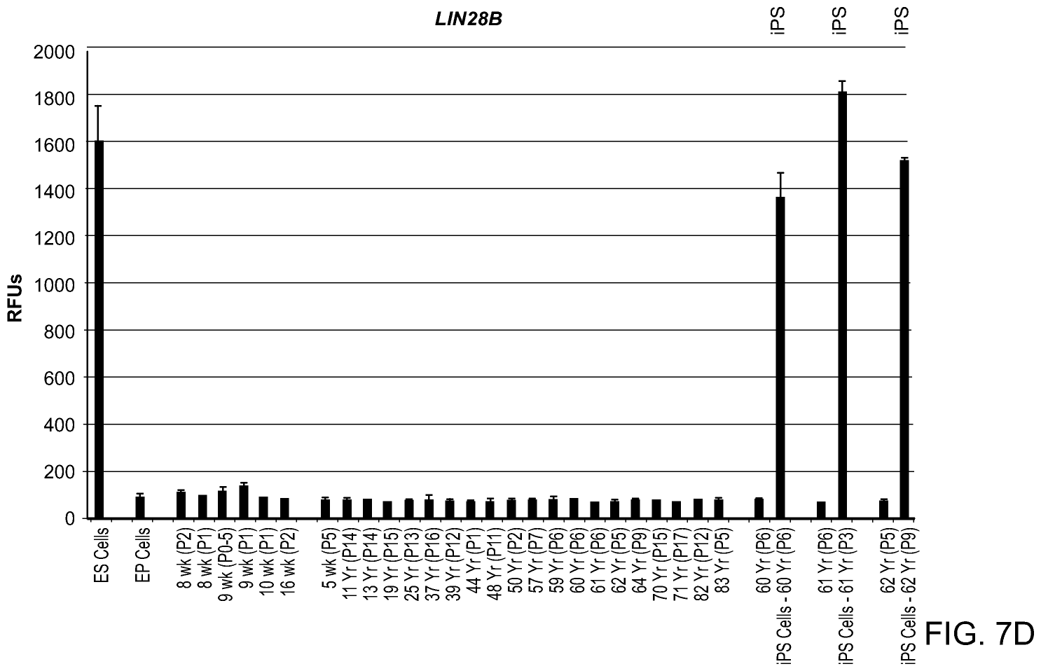

To validate the markers in human development, we utilized early passage dermal fibroblasts of the upper arm beginning with the onset of fetal development (eight weeks of gestation) all cultured in identical conditions. Illumina gene expression bead array-based data shown in FIG. 7 showed that the genes COX7A1, NAALADL1, and PLPP7 were again induced beginning with 8 weeks of development, perhaps the most striking marker being COX7A1 which appeared to progressively increase in expression throughout fetal and postnatal development, leveling off in adulthood. LIN28B was expressed at the highest levels in ES cells, with low but detectable levels in the embryonic progenitors and in fibroblasts from early fetal development, but not in adult-derived cells. All patterns of EFT gene expression described in the present invention were effectively reprogrammed from an adult pattern back to an embryonic pattern of gene expression in aged fibroblast-derived iPS cells. For example, as seen in FIG. 7, the normal skin fibroblasts derived from 60, 61, and 62 year-old donors expressed relatively high levels of COX7A1, NAALADL1, and PLPP7 mRNA, while those transcriptionally reprogrammed to pluripotency (designated “iPS Cells-60 Yr,” “iPS Cells-61 Yr,” “iPS Cells-62 Yr” in FIG. 7) expressed a embryonic (pre-fetal or prenatal) pattern. Reprogramming to iPS cells was performed as described herein, briefly, adult-derived COX7A1, NAALADL1, and PLPP7-expressing human fibroblasts were reprogrammed to pluripotency by plating the cells on Matrigel-coated 6-well plates and transfecting the mRNAs for OCT4 (POU5F1), SOX2, KLF4, MYC and LIN28 (Day 0). On days 1-12, the cells were again transfected with the OCT4 (POU5F1), SOX2, KLF4, MYC and LIN28 cocktail. By day 12˜day 14 reprogramming to pluripotency was verified with live-staining Tra-1-60 antibody and iPS cell colonies were picked.

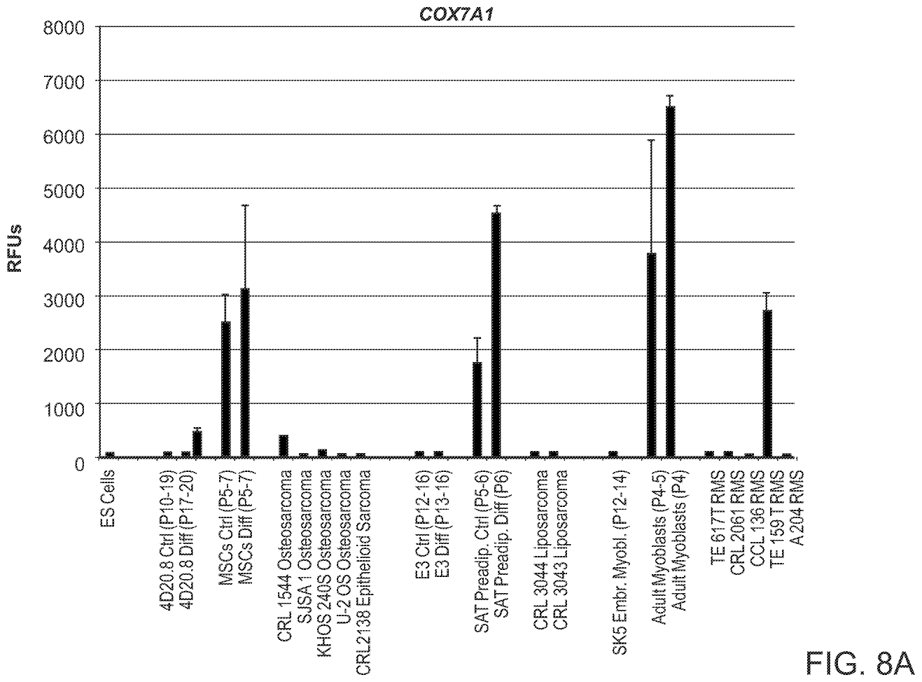

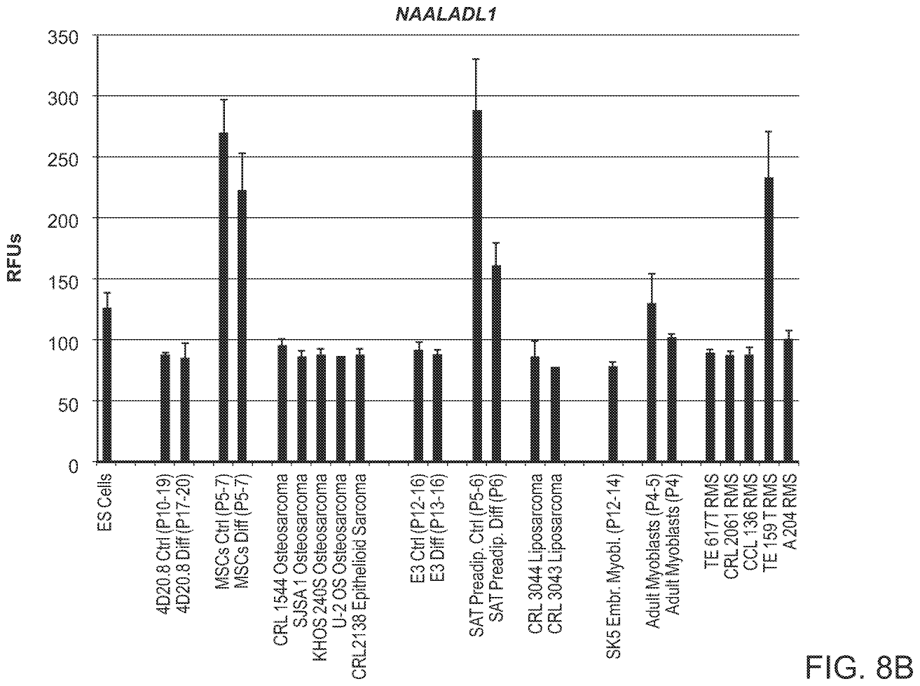

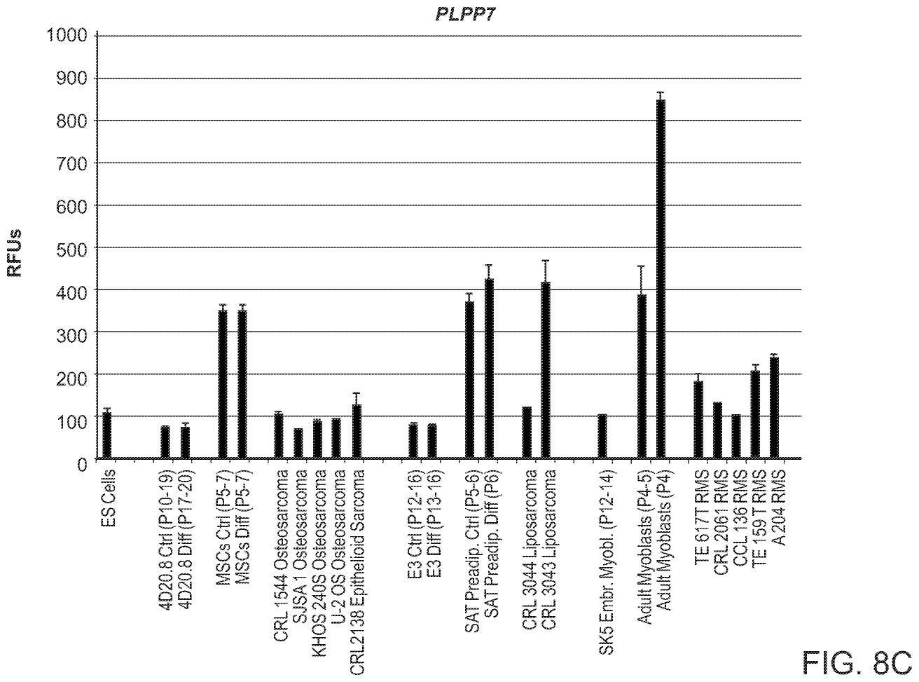

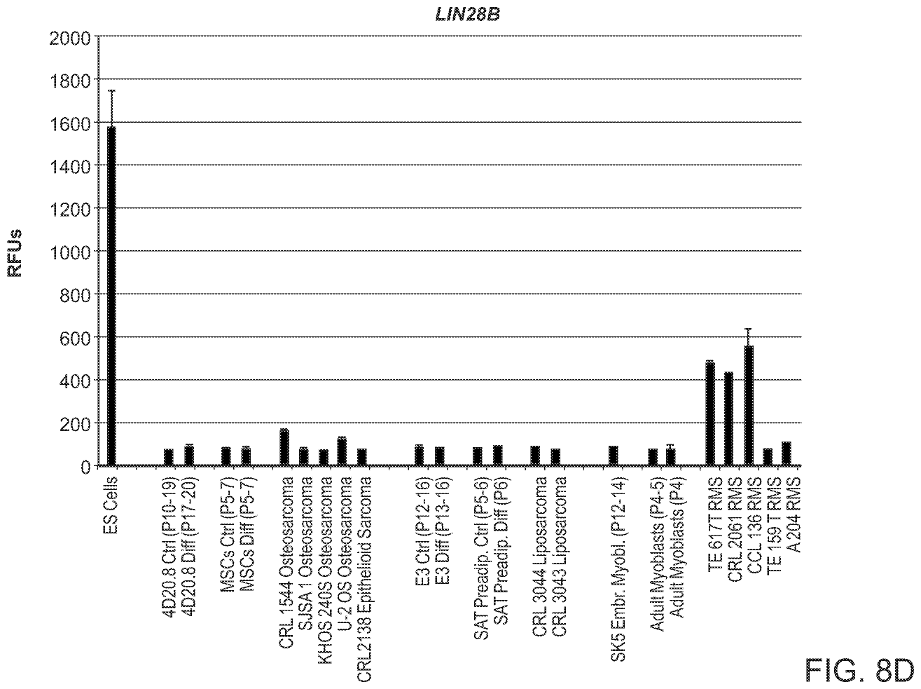

We next examined the expression of the genes in three types of sarcomas (osteosarcoma, liposarcoma, and rhabdomyosarcoma) (see, FIG. 8). Embryonic progenitors capable of osteochondral differentiation such as the line 4D20.8 showed no evidence of COX7A1, NAALADL1, or PLPP7 expression either in the progenitor state or in the differentiated state despite expressing high levels of osteochondral markers. In contrast, adult-derived MSCs expressed COX7A1, NAALADL1, and PLPP7 before and after differentiation. In osteosarcomas, the lines generally showed an embryonic pattern of gene expression. For instance, 4/5 osteosarcoma cell lines showed little to no detectable COX7A1 transcript. Similarly, an embryonic progenitor cell line capable of adipogenic differentiation designated E3 did not induce COX7A1, NAALADL1, or PLPP7 despite expressing robust markers of adipocte differentiation, while adult-derived subcutaneous adipose tissue (SAT) preadipocytes expressed COX7A1, NAALADL1, and PLPP7 in both the relatively undifferentiated as well as fully differentiated adipocytes. However, in two liposarcoma cell lines studied, the markers appeared to reflect an embryonic pattern of gene expression, for example, both liposarcoma lines expressing no or very low levels of COX7A1 transcript. Lastly, five rhabdomyosarcoma cell lines were similarly studied in comparison to an embryonic myoblast progenitor cell line designated SK5, and adult-derived myoblasts. COX7A1, previously described as being highly expressed in skeletal and cardiac myocytes, was expressed at high levels in the adult-derived cells, but not in the embryonic progenitor line SK5, and was not expressed or expressed at low levels in 4/5 of the rhabdomyosarcoma cell lines and LIN28B was expressed at relatively high levels in 3/5 of the lines.

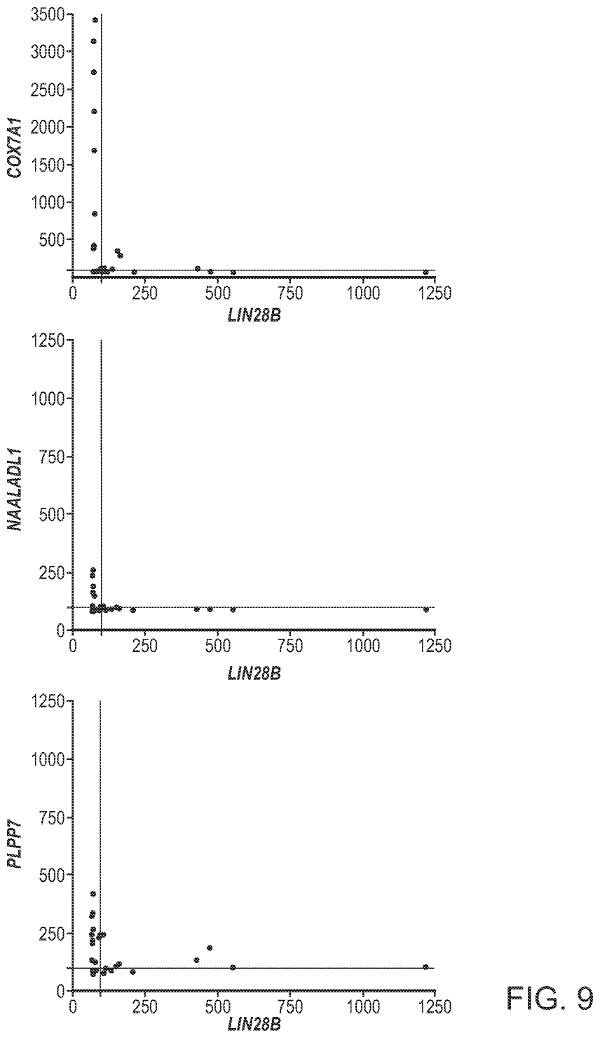

As further evidence of the validation of the genes COX7A1, NAALADL1, and PLPP7 as markers of fetal transition as well as the gene LIN28B as a marker of the embryonic state, the three pairs of genes (i.e. LIN28B vs COX7A1, NAALADL1, or PLPP7) were identified with strong inverse agreement in diverse sarcoma cell lines, that is, when one gene was expressed the other gene was not, or both genes were not expressed. Expression was defined as an XYZ of greater than 100 XYZs. The pairs of genes are shown in FIG. 9 where the shaded regions highlight the strong inverse agreement of the genes. The percent of inverse agreement between LIN28B and COX7A1 is 83.3% (95%CI: 66.4-92.7); LIN28B and NAALAD1 is 100% (95%CI: 88.6-100); and LIN28B and PLPP7 is 73.3% (95%CI: 55.6-85.8).
Example 2. Use of the embryonic marker PCDHB2 and the fetal-adult marker COX7A1 to screen for hormonal agents capable of causing iTM in hES cell-derived clonal EP cell lines. The clonal EP cell line designated 4D20.8 was serially passaged in the relatively undifferentiated state as described (West et al, Regen. Med. (2008) 3(3), 287-308) incorporated herein by reference. In the relatively undifferentiated progenitor state, the line 4D20.8 expresses markers consistent with cells in the embryonic (pre-fetal) state such as relatively high levels of PCDHB2, but undetectable levels of COX7A1 similar to the hES cells from which they were derived. After serial passaging in vitro to P36 which took longer than 8 weeks, there was no induction of COX7A1 expression observed and little if any loss of PCDHB2 expression. Therefore we conclude that serial passaging and the simple passage of time is not sufficient by itself to mature EP cells into the fetal transition. Similarly, differentiating the embryonic progenitor cell line 4D20.8 in micromass conditions for 62 days in the presence of TGFb3 and BMP4 similarly did not result in an induction in COX7A1 and only a partial reduction in PCDHB2 expression.

The line 4D20.8 is therefore useful in screening for factors capable of promoting iTM. 4D20.8 or other embryonic progenitors are exposed to hormonal factors including the following pools and RNA is harvested from the cells after 2,4, or 6 weeks to assay for the induction of adult markers such as COX7A1 or reduction in embryonic markers such as PCDHB2. Hormonal factors screened are:
Pituitary Pool: Thyroid-stimulating hormone (TSH) 1 nM, Adrenocorticotrophic hormone (ACTH) 5 nM, Luteinizing hormone (LH) 100 ng/ml, Follicle-stimulating hormone (FSH) 10 ng/ml, Somatotrophin/growth hormone (GH) 1 ng/ml, Prolactin (PRL) 50 ng/ml, Melanocyte-stimulating hormone (MSH) 1 ng/ml, Oxytocin 10 nM, Arginine 0.5 mM, vasopressin 1 uM.
Hypothalamic Pool: Thyrotrophin releasing hormone (TRH 1 uM), Corticotrophin releasing hormone (CRH) 100 nM, Arginine vasopressin (AVP) 1 uM, Gonadotrophin releasing hormone (GnRH) 1 ug/ml, Growth hormone releasing hormone (GHRH) 10 nM, Somatostatin 1 nM, Prolactin releasing factor (PRF) 10 nM, Dopamine 50 uM.
Thyroid/Parathyroid Pool: T3 2 nM, T4 10 ng/ml, parathyroid hormone 50 nM.
Pancreatic Pool: Insulin 5 ng/ml, glucagon 5 ug/ml, somatostatin 1 nM, pancreatic polypeptide 2 ng/ml.
Adrenal Pool: Cortisol 10 nM, Aldosterone 2 ng/ml, Dehydroepiandrosterone 10 uM, epinephrine 1 uM, norepinephrine 1 uM.
Gonadal Pool: Testosterone 50 ng/ml, estrogen 5 nM, progesterone 100 nM. Placental Pool: Chorionic gonadotropin lng/ml, Human chorionic somatommotropin (hCS),
Human chorionic corticotropin (hCACTH), chorionic thyrotropin (hCT).
Pools 1+2+3
Pools 1+2 +5
Pools 1+2+3 +5
Example 3. Novel RNAs differentially expressed in embryonic vs adult cells as determined by RNA-seq.

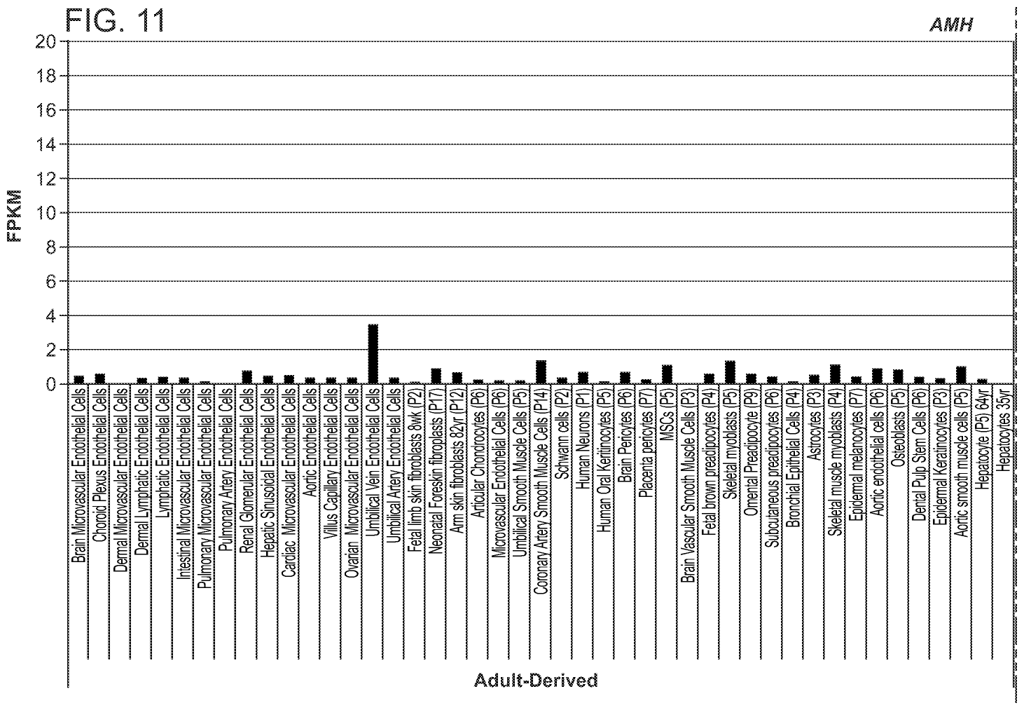

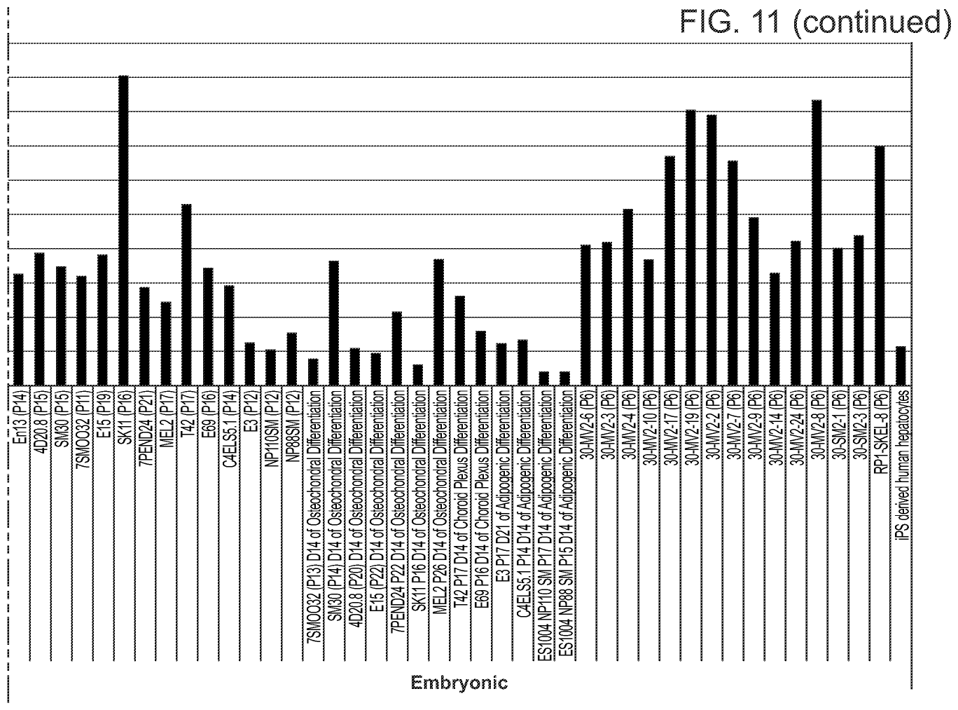

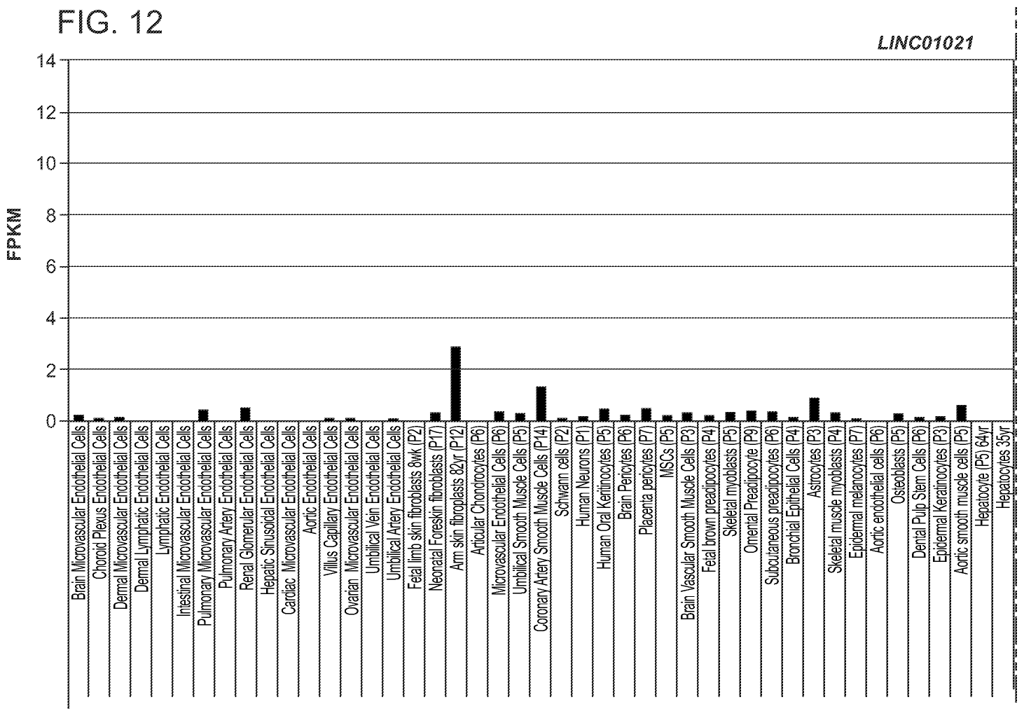

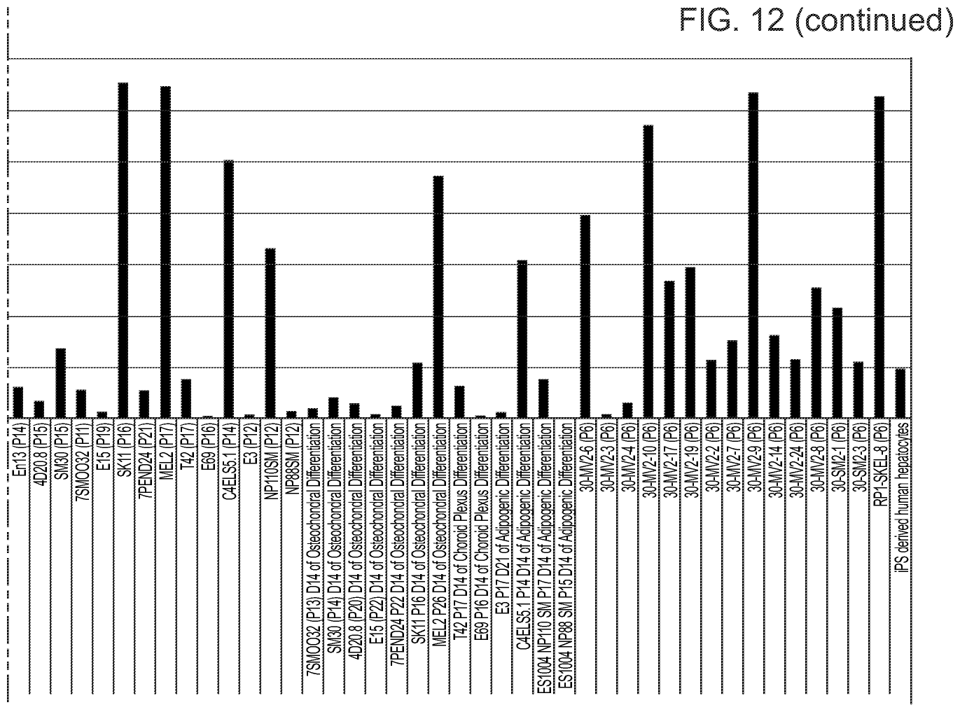

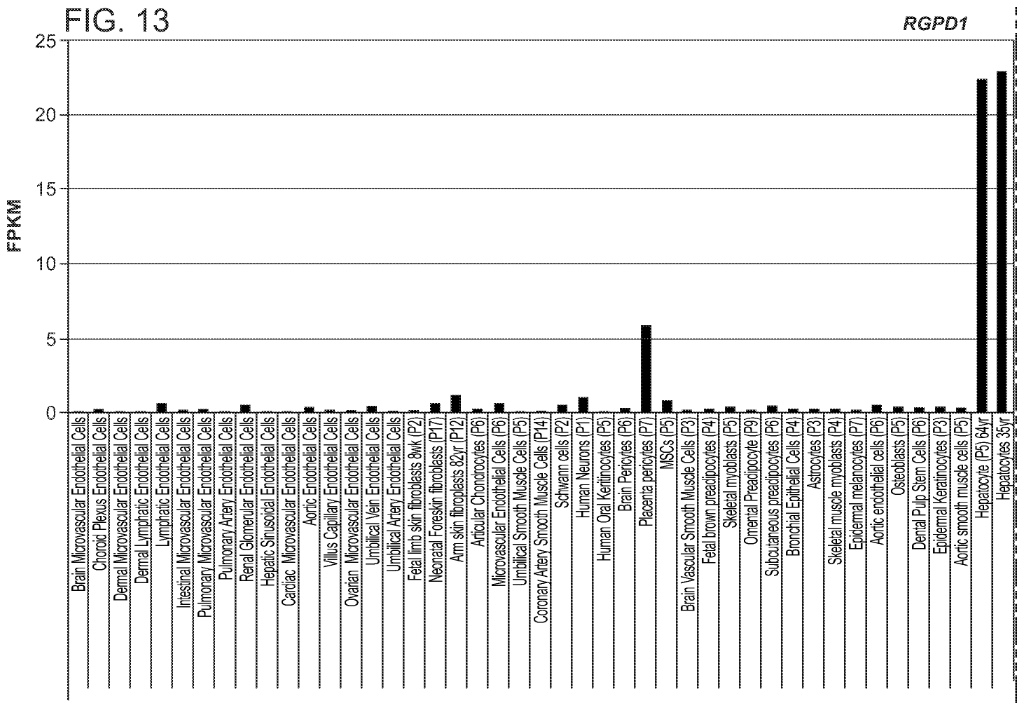

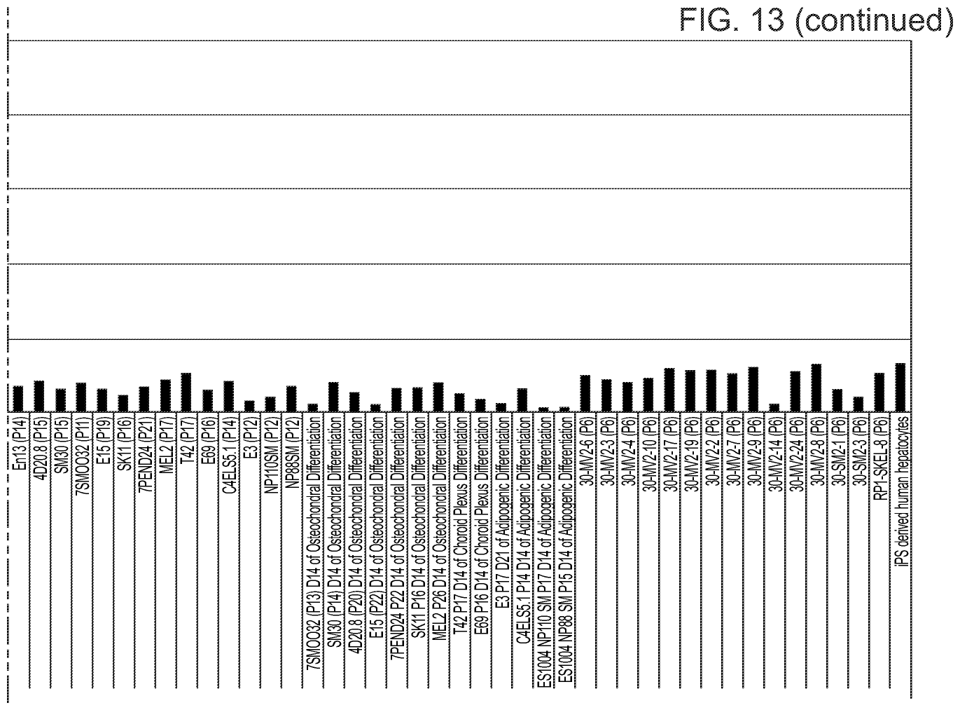

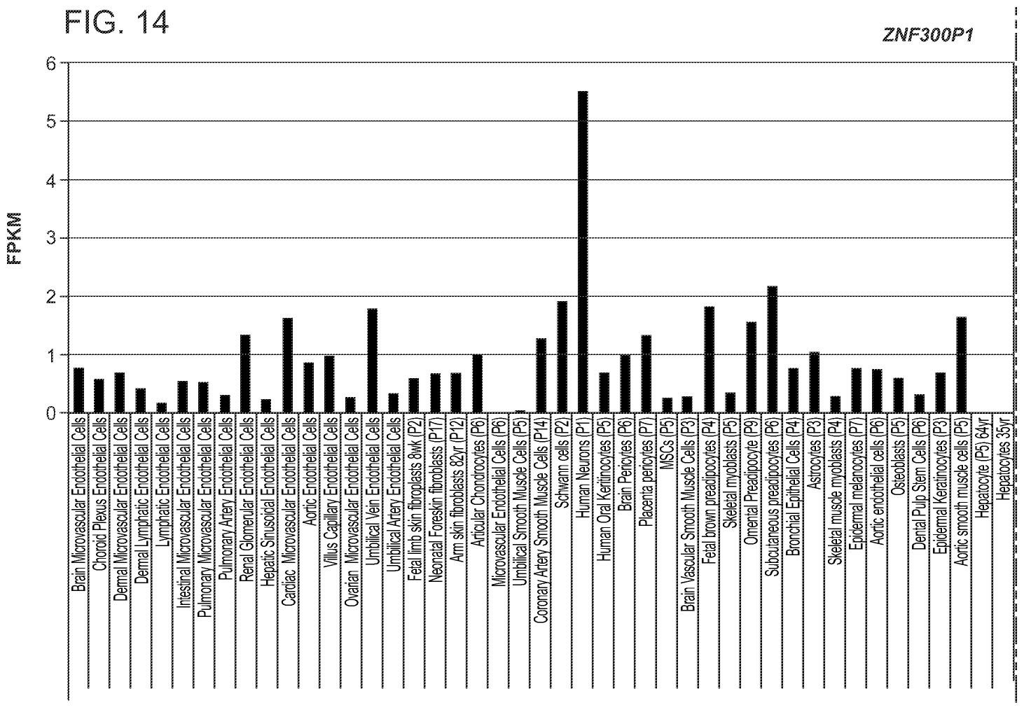

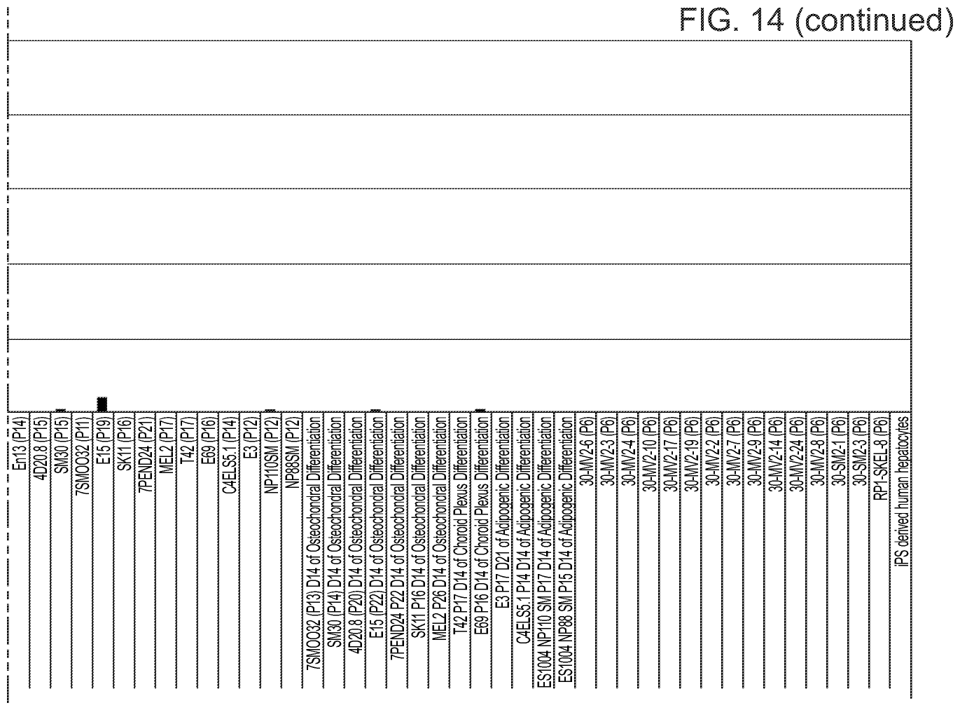

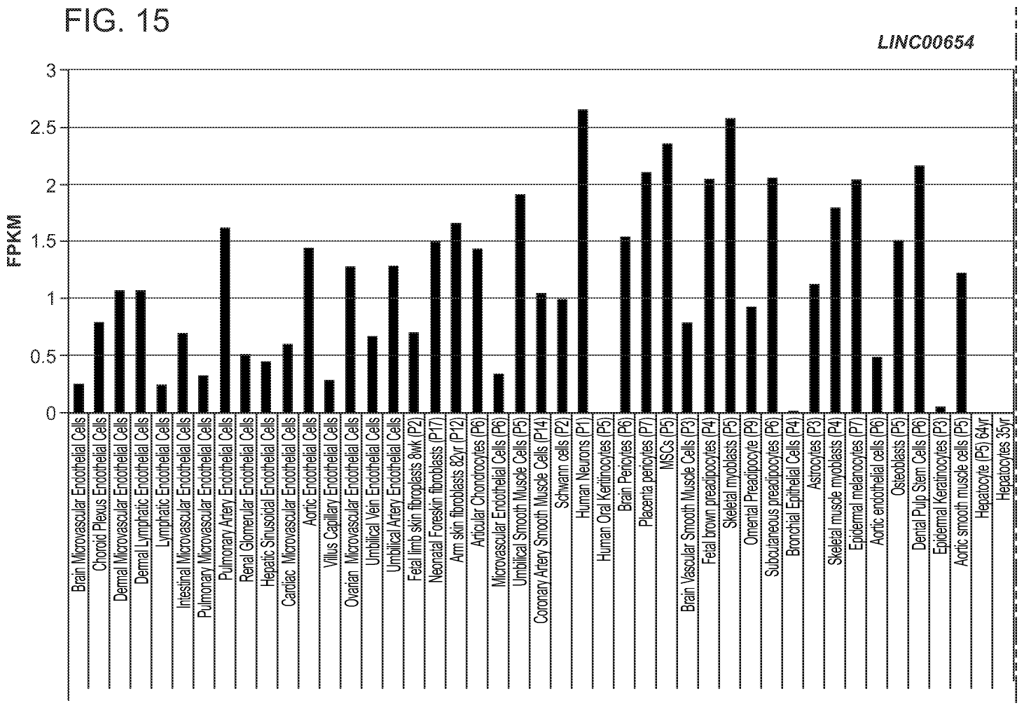

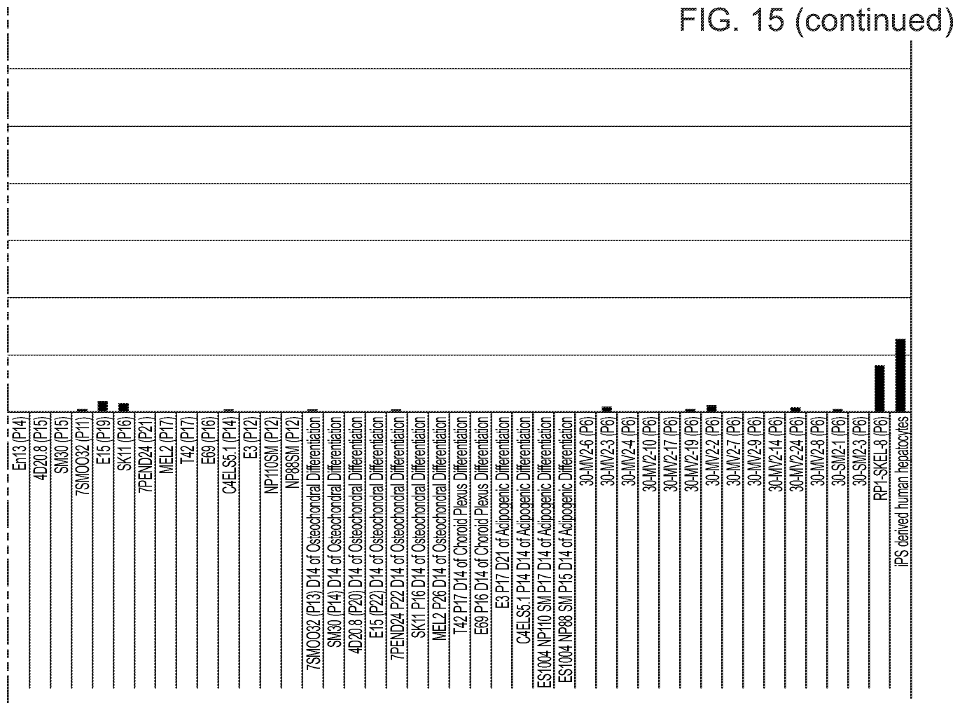

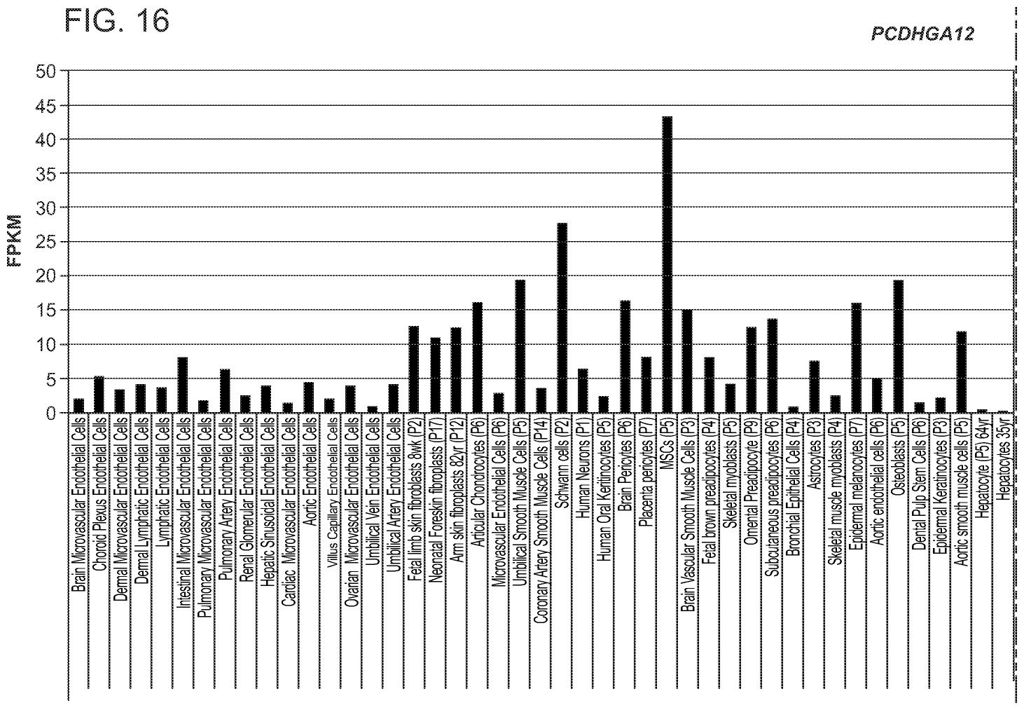

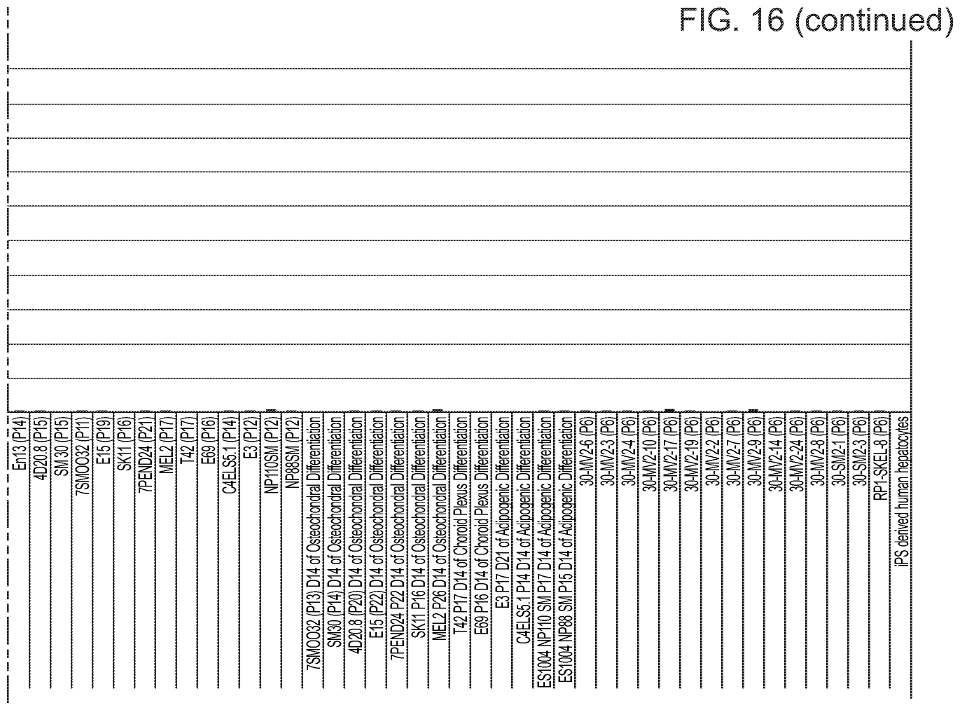

RNA sequencing was performed on Illumina HiSeq4000 platform to a depth of a minimum of 25 million 100 base pairs paired-end reads. Data were processed using Tuxedo suite protocol (Trapnell C. et al., Differential gene and transcript expression analysis of RNA-seq experiments with TopHat and Cufflinks; Nat Protoc. 2012 Mar 1;7(3):562-78. doi: 10.1038/nprot.2012.016.). Alignment data are visualized using Integrative Genomics Viewer (Robinson, J. et al., Integrative Genomics Viewer, Nature Biotechnology 29, 24-26 (2011)) with data presented and values FPKM. Novel embryonic and adult-specific markers are shown in FIG. 10. As shown in FIGS. 11, the growth factor AMH was expressed at markedly higher levels in embryonic cells. As shown in FIG. 12, the noncoding RNA LINC01021 was expressed at higher levels in embryonic progenitors compared to adult cells, and the RNA decreased in prevalence with the differentiation of the progenitors. As shown in FIG. 13, the transcript RGPD1 was generally detected at higher levels in embryonic compared to adult cells with the exception of adult hepatocytes which expressed markedly higher levels of RGPD1 transcript. As shown in FIG. 14, the transcript from ZNF300P1 was generally expressed in adult cell types (not in hepatocytes, however), but not in most embryonic progenitors. As shown in FIG. 15, the transcript LINC00654 was generally expressed in adult, but not embryonic progenitors. As shown in FIG. 16, the transcript for PCDHGA12 was markedly expressed in adult-derived cells compared to embryonic progenitor cells. As with the other genes described herein, increasing the embryonic pattern would facilitate iTR and increasing the adult pattern would facilitate iTM and iCM.
Example 4. Differential expression of the clustered protocadherin genes in embryonic vs adult cells as determined by RNA-seq.

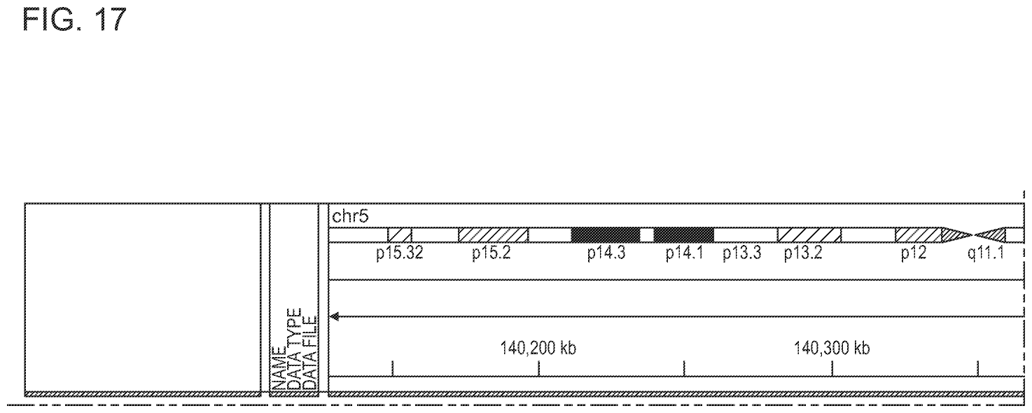

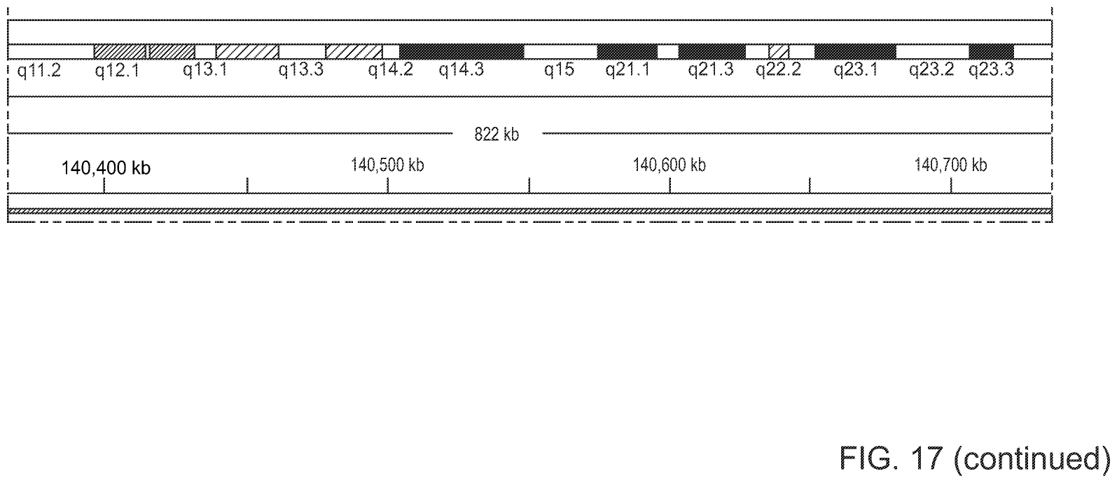

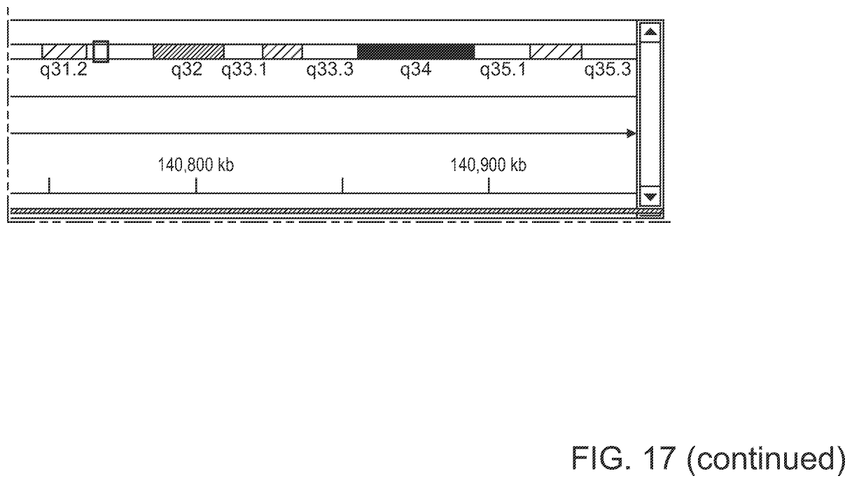

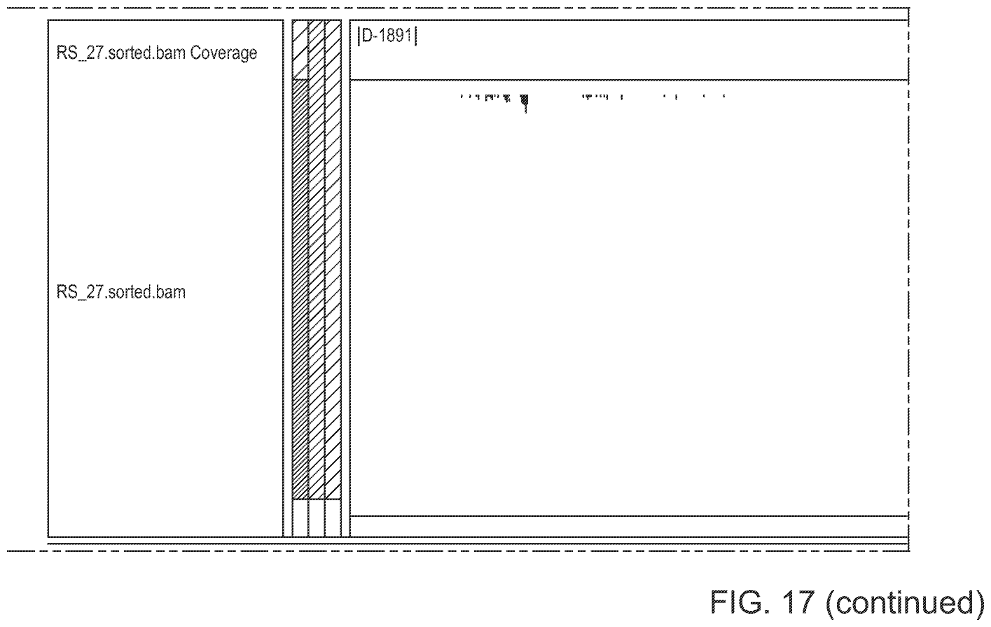

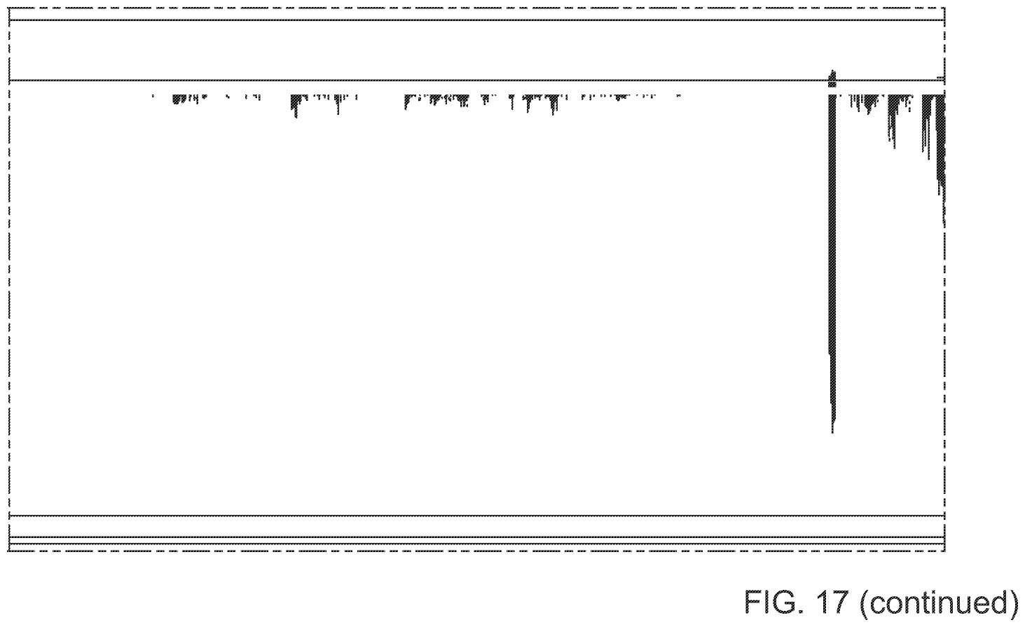

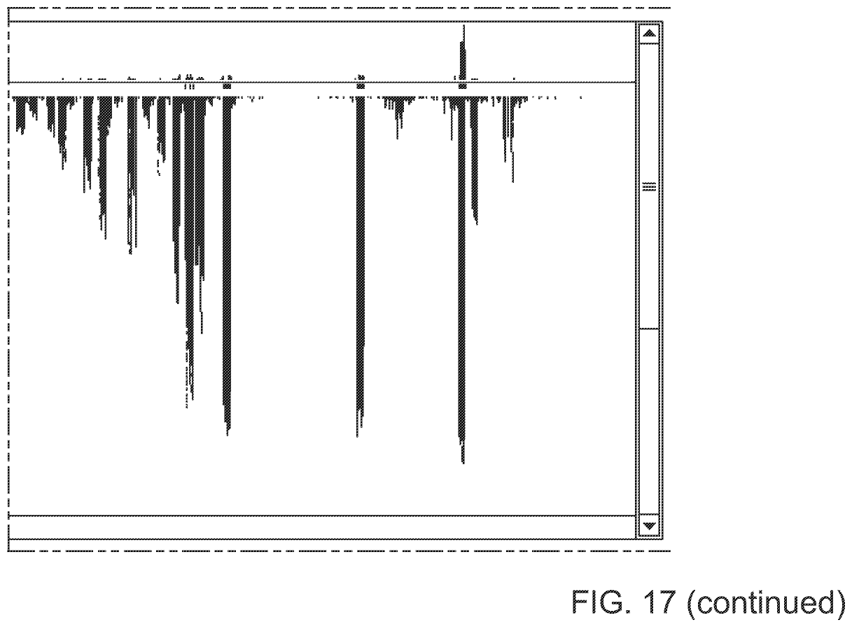

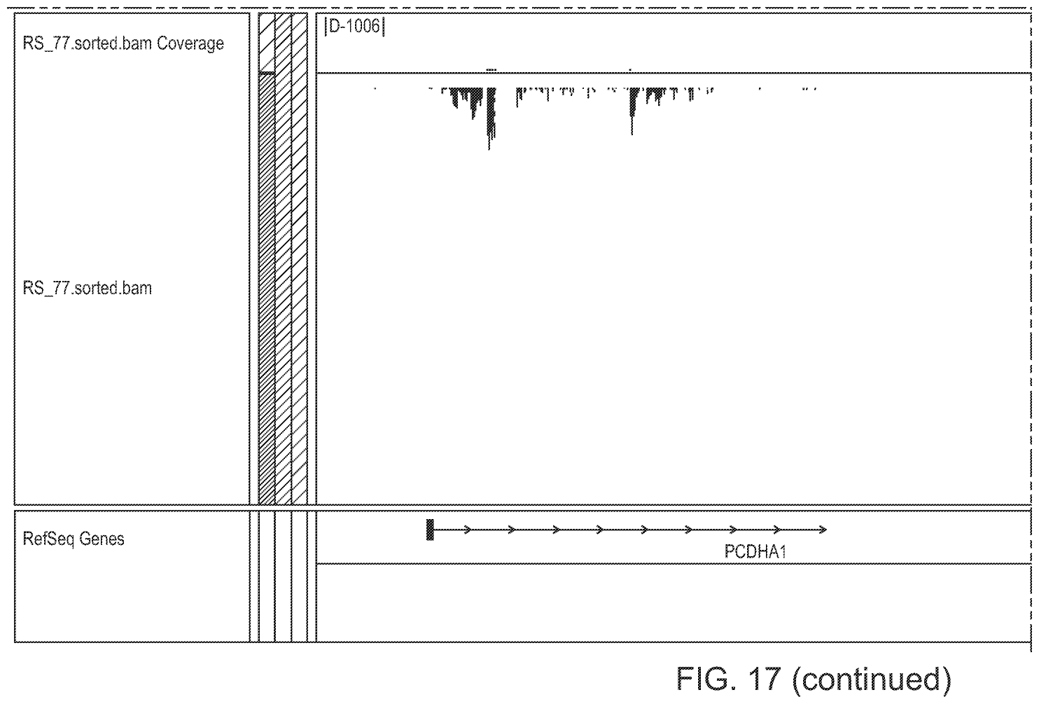

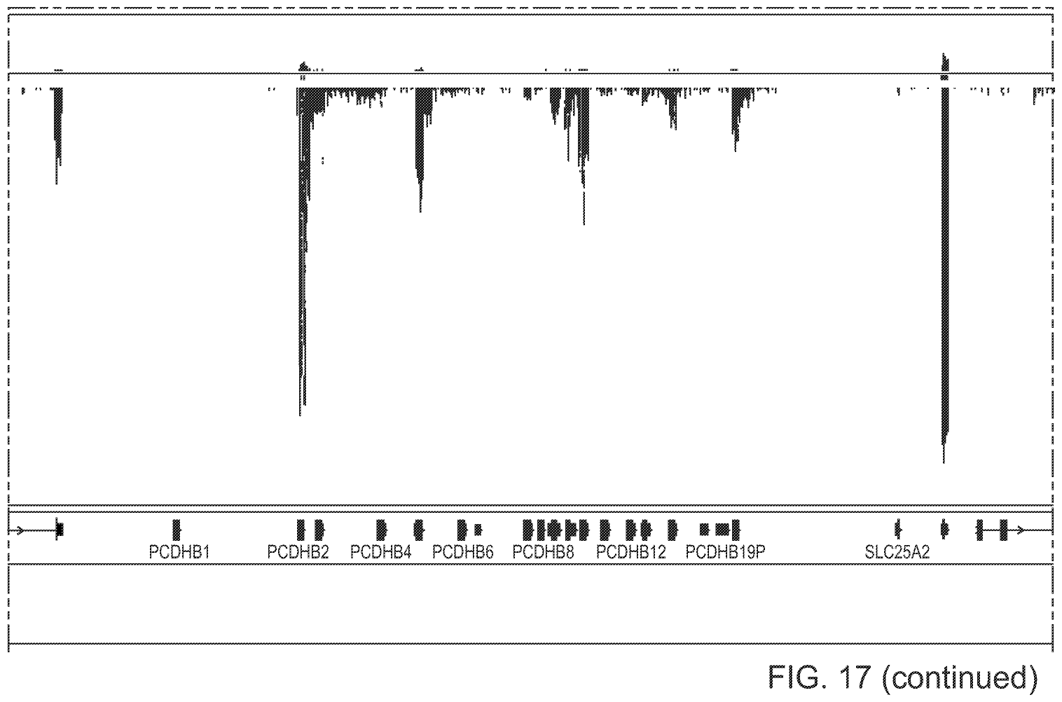

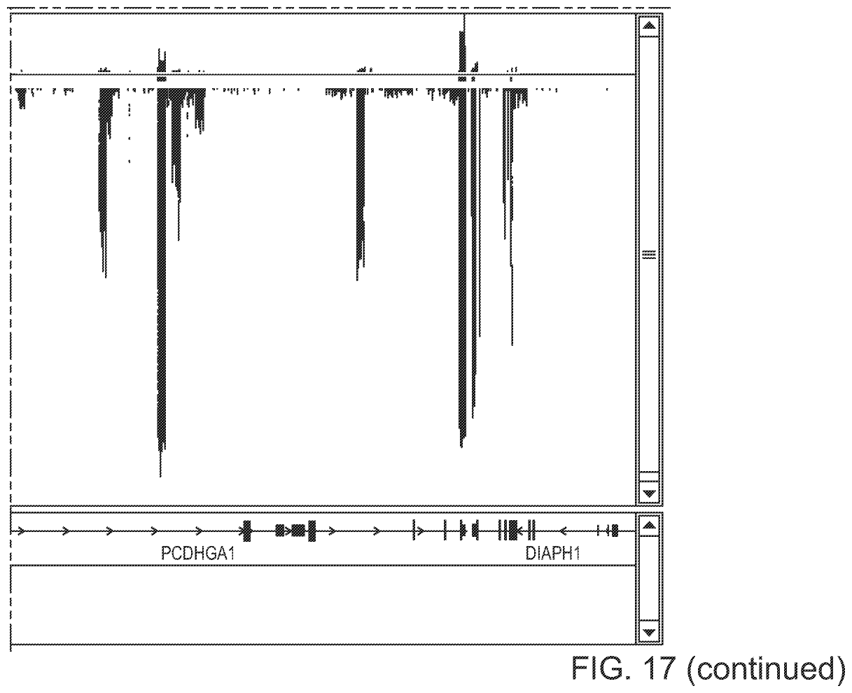

RNA sequencing was performed on Illumina HiSeq4000 platform to a depth of a minimum of 25 million 100 base pairs paired-end reads. Data were processed using Tuxedo suite protocol (Trapnell C. et al., Differential gene and transcript expression analysis of RNA-seq experiments with TopHat and Cufflinks; Nat Protoc. 2012 Mar 1;7(3):562-78. doi: 10.1038/nprot.2012.016.). Alignment data are visualized using Integrative Genomics Viewer (Robinson, J. et al., Integrative Genomics Viewer, Nature Biotechnology 29, 24-26 (2011)). As shown in FIG. 17, numerous striking differences in reads from exons in the clustered protocadherin locus in embryonic vs adult cell types was observed, with embryonic cells generally expressing more transcripts from the alpha and beta genes, and adult cells, more from the gamma cluster genes. As with the other genes described herein, increasing the embryonic pattern would facilitate iTR and increasing the adult pattern would facilitate iTM and iCM.
Example 5. Screening for factors capable of inducing iTR using the markers of the present invention.

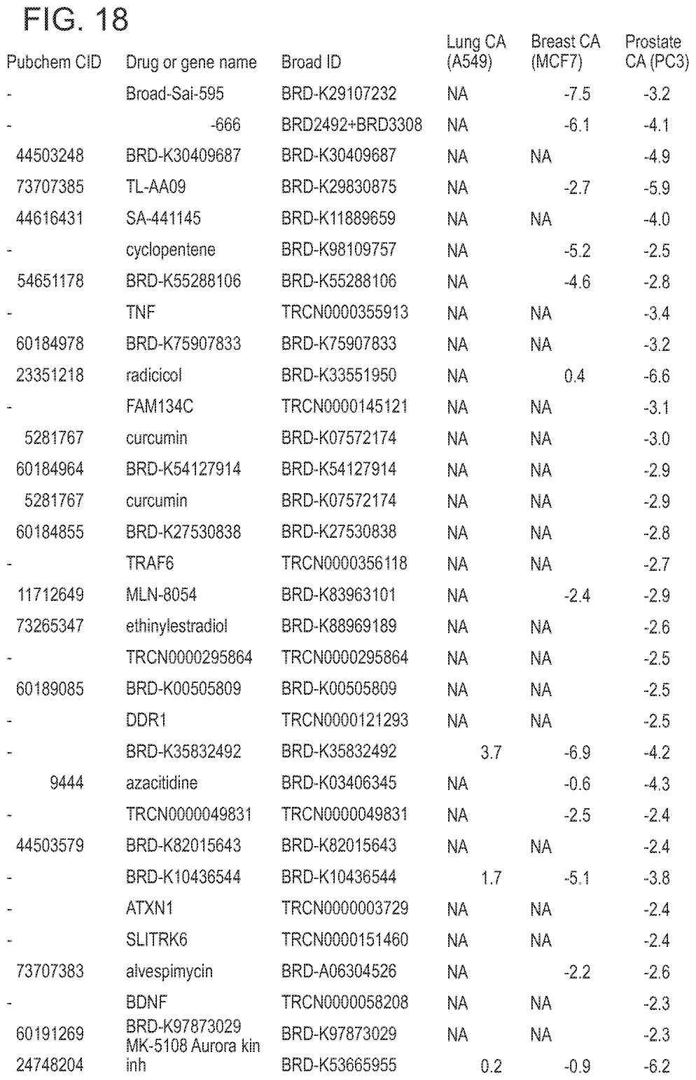

Lung (A549), breast (MCF7), and prostate (PC3) cancer cell lines were exposed to diverse factors including biologically active small molecules as well as RNAi directed to specific genes and the relative reduction of levels of COX7A1 transcript was used as a marker of iTR. As shown in FIG. 18, HSP90 inhibitors such as radicocol and alvespimycin reduce COX7A1 expression, as does RNAi directed towards HIF1A, curcumin, azacitidine (5-aza-2′deoxycytidine), and inhibitors of Aurora B/C kinase such as GSK1070916 and MK-5108, a highly selective Aurora-A kinase inhibitor.
Example 6. Screening for factors capable of inducing iCM using the markers of the present invention.

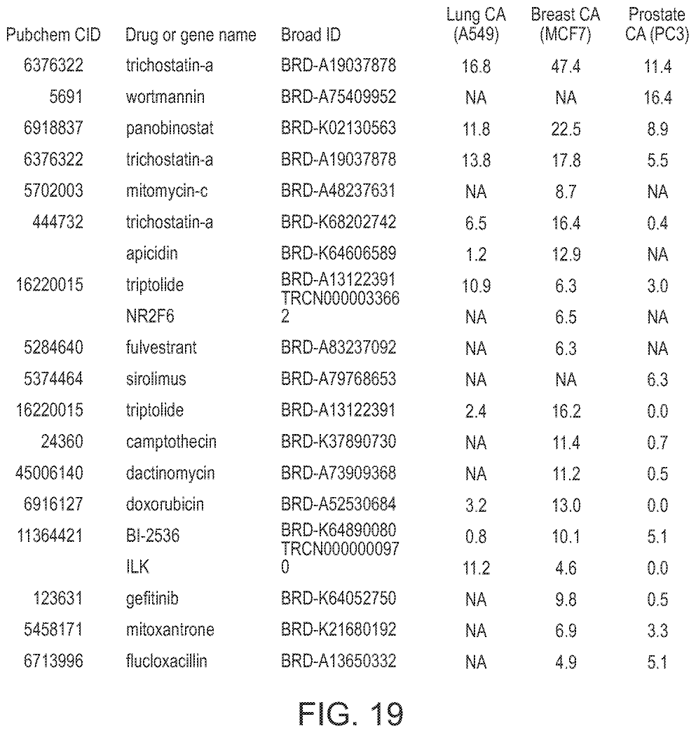

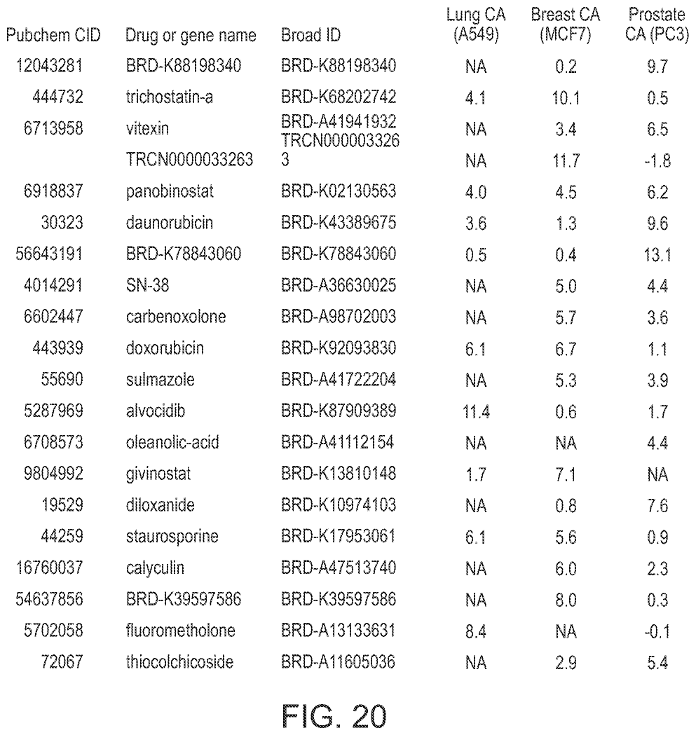

Lung (A549), breast (MCF7), and prostate (PC3) cancer cell lines were exposed to diverse factors including biologically active small molecules as well as RNAi directed to specific genes and the relative increase in levels of COX7A1 transcript was used as a marker of iCM. As shown in FIG. 19-20, numerous histone deacetylase inhibitors including trichostatin-A, panobinostat, apicidin, and givinostat increased COX7A1 expression in the cancer cell lines as well as triptolide and BI 2536, a Potent and Selective Inhibitor of Polo-like Kinase 1 that is reported to have anti-tumor activity, and wortmannin, a non-specific, covalent inhibitor of phosphoinositide 3-kinases, and dactinomycin, flucloxacillin, gefitinib, mitoxantrone, vitexin, daunorubicin, carbenoxolone, sulmazole, alvocidib, SN-38, teniposide, calyculin, staurosporine, and doxorubicin. These agents are therefore therapeutically useful in the treatment of cancer, in particular those cancers determined using the markers described herein, of displaying an embryonic pattern of gene expression.
Example 7. Screening for optimum conditions for iTR in differentiated fetal or adult-derived cells using iPS cell factors but without reverting the cells to pluripotency

As shown in Example 1 above, transcriptional reprogramming of adult-derived differentiated cells to pluripotency such as through the use of mRNA for the genes OCT4, SOX2, KLF4, MYC and LIN28A causes a pattern of multiple gene expression changes associated with iTR as described in the present invention. Therefore, the cocktail itself could be used not only in vitro as shown in Example 1, but also in vivo as described herein as an iTR agent to increase tissue regeneration in the context of degenerative disease. However, because the administration of such a cocktail of reprogramming factors for an extended period of time in vivo could have the inherent risk of the reversion of cells in vivo to pluripotency with subsequent appearance of teratomas, abnormal ectopic tissues, or even malignancies, there is a need to identify methods of safely generating an iTR pattern of gene expression in fetal and adult-derived cells in vitro and in vivo without reprogramming the cells completely to pluripotency. Such a protocol would provide a method for the epigenetic reprogramming of said fetal or adult-derived differentiated cells expressing markers of cells that have traversed the EFT such as the expression of COX7A1, CAT, and NAALADL1, such that the promoters of aforementioned genes are methylated to a relatively greater extent, thereby reverting the cells to a pre-fetal (i.e. iTR) pattern of gene expression but not reverting the cells to pluripotency or altering their fate from the primary germ layer (i.e. mesodermal, ectodermal, endodermal, neural crest) that they were before the administration of the exogenous agents. Ideally, such a screen will additionally identify downstream regulators capable of generating iTR without the use of the iPS cell-generating factors to further reduce the risk of malignancy in a target tissue.

This optimized protocol for the identification of such conditions is identified as follows. Adult-derived normal skin fibroblasts, and separately, the identical fibroblasts immortalized with lentivirus expressing the catalytic component of telomerase (TERT) and selected by the use of neomycin (G418) resistance, are each further infected with lentiviral vectors expressing the genes OCT4, SOX2, KLF4, NANOG, ESRRB, NR5A2, CEBPA, MYC, LIN28A and LIN28B alone and in diverse combinations, and in diverse combinations with small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDAC10), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDAC11) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycytidine (trade name Vida za and Azadine); vitamin C which can inhibit DNA methylation, increase Tet1 which increases 5hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1); agents that promote the activity of the HIF1 transcription complex including but not limited to Quercetin; RAR agonists including but not limited to AM580, CD437, and TTNPB; agents that mimic hypoxia including but not limited to Resveratrol;, agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or CAT, or other inhibitors of iTR as described herein, and/or assaying for increased expresson of PCDHB2 or AMH or other activators or iTR as described herein, or in injured or diseased tissues in vivo, or in modulating the lifespan of animals in vivo.

MDW cells (normal as well as TERT-immortalized) were plated in vitro in culture seeded at 1×10⁵cells/well in 12-well plates with DMEM media containing 10% FBS. Polybrene was added to the media (0.8 ul/ml of Polybrene (at a stock of 10 ug/ul) to the 1 mL of virus/media for a final concentration of 8 ug/mL). Virus was added at 1 ml per well. Control type 1 (“empty virus”=GFP only expressing virus); use one well for Control type 2 (not infected). Since there were two different types of cells (with TERT and without TERT), there were two sets of controls. Media was gently swirled to mix and cover the cells. Plates of cells with the virus were returned to the incubator at 37° C. and 5% CO₂ overnight. At one day after transduction, the virus media was removed and replaced with normal DMEM media supplemented with 10% FBS and fed every other day, then RNA harvested for RNA sequencing.

In vitro assays for iTR patterns of expression of the genes COX7A1, CAT, and NAALADL1 as well as gene expression or protein markers of pluripotency including DNMT3B, and HELLS or Tra-1-60, Tra-1-81, and SSEA4 respectively are performed to optimize global patterns of iTR gene expression without reverting the target cells to pluripotency. Examples of individual agents and combinations of agents screened are: OCT4, SOX2, KLF4, MYC and LIN28A; OCT4; KLF4; OCT4, KLF4; OCT4, KLF4, LIN28A; OCT4, KLF4, LIN28B; SOX2; MYC; NANOG; ESRRB; NT5A2; OCT4, SOX2, KLF4, and LIN28A; OCT4, SOX2, KLF4, and LIN28B; OCT4, KLF4, MYC and LIN28A; and each of the preceeding combinations of agents together with 0.25 mM NaB, 5 μM PS48 and 0.5 μM A-83-01 during the first four weeks, followed by treatment with 0.25 mM sodium butyrate, 5 μM PS48, 0.5 μMA-83-01 and 0.5 μM PD0325901 each of which is assayed at 0, 1, 2, 4, 7, 10, and 14 days for markers of global modulation of iTR gene expression.

The resulting optimized conditions when combined with elevated expression of the catalytic component of telomerase such as through the transient expression of the gene TERT in cases where telomere length is limiting, provides a means of inducing an iTR pattern of gene expression including but not limited to decreased COX7A1, PLPP7, and NAALADL1 gene expression in fetal or adult cells in vitro or in vivo to induce tissue regeneration.

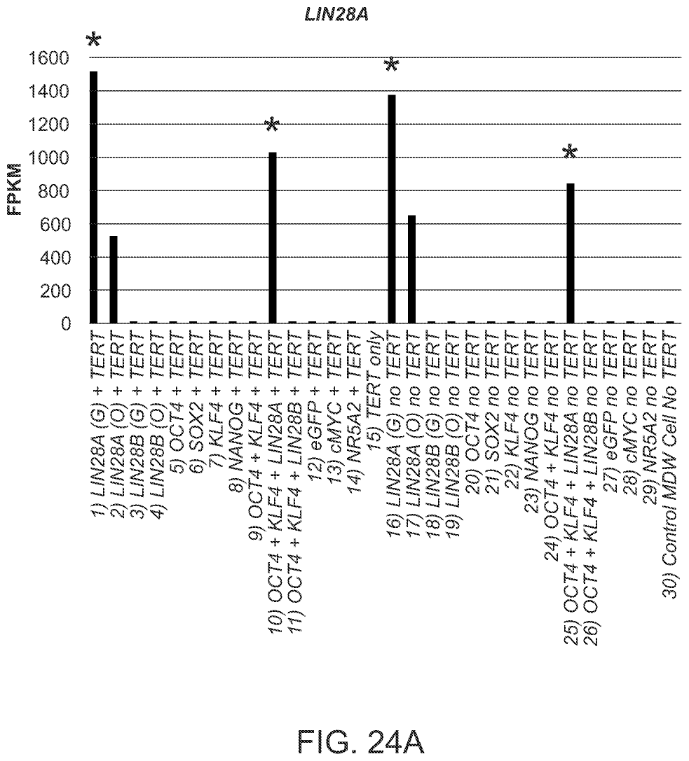

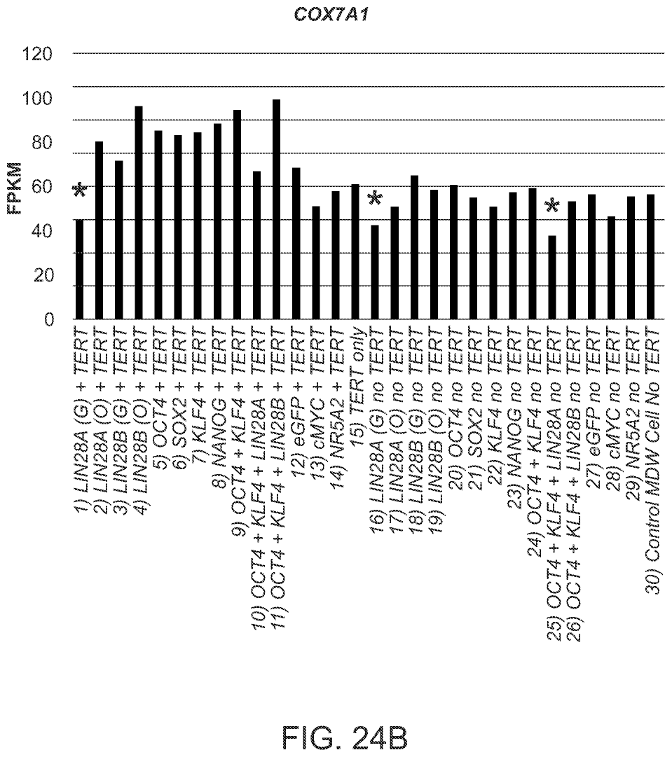

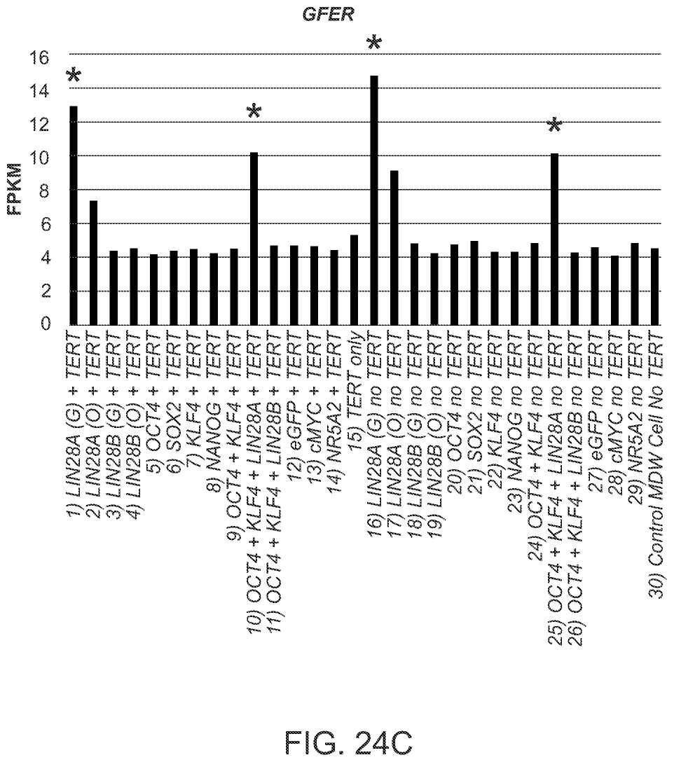

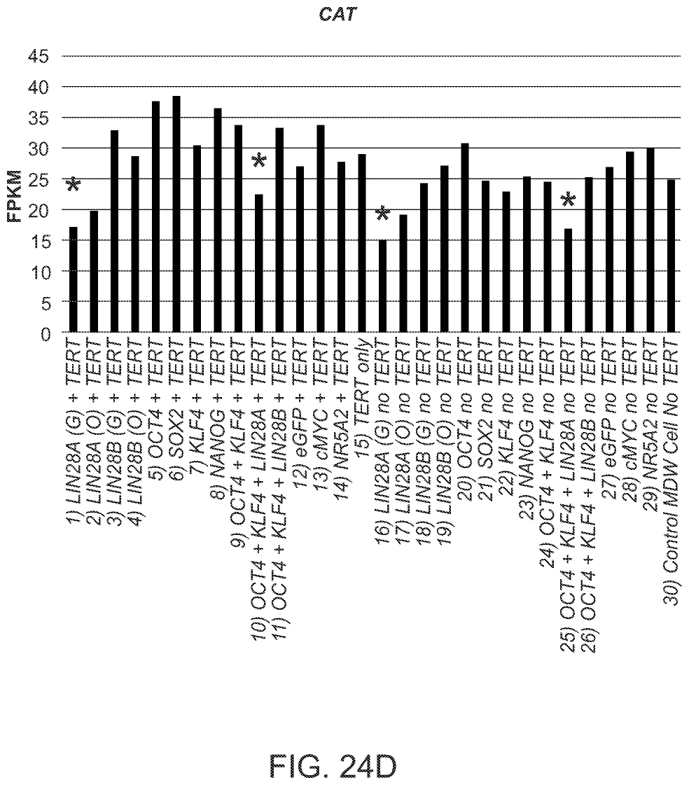

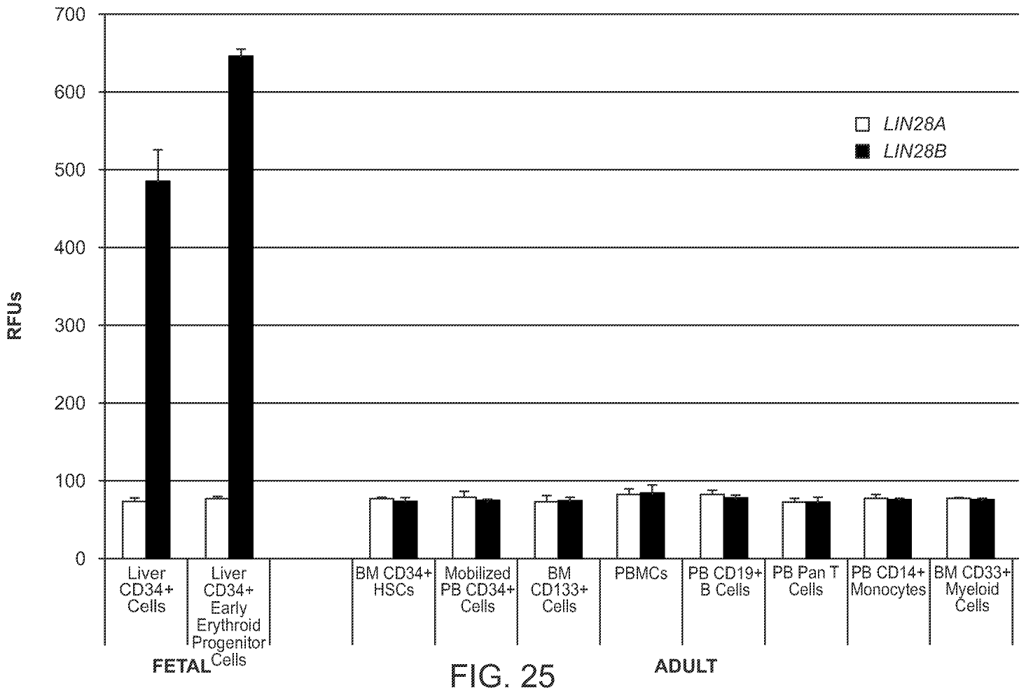

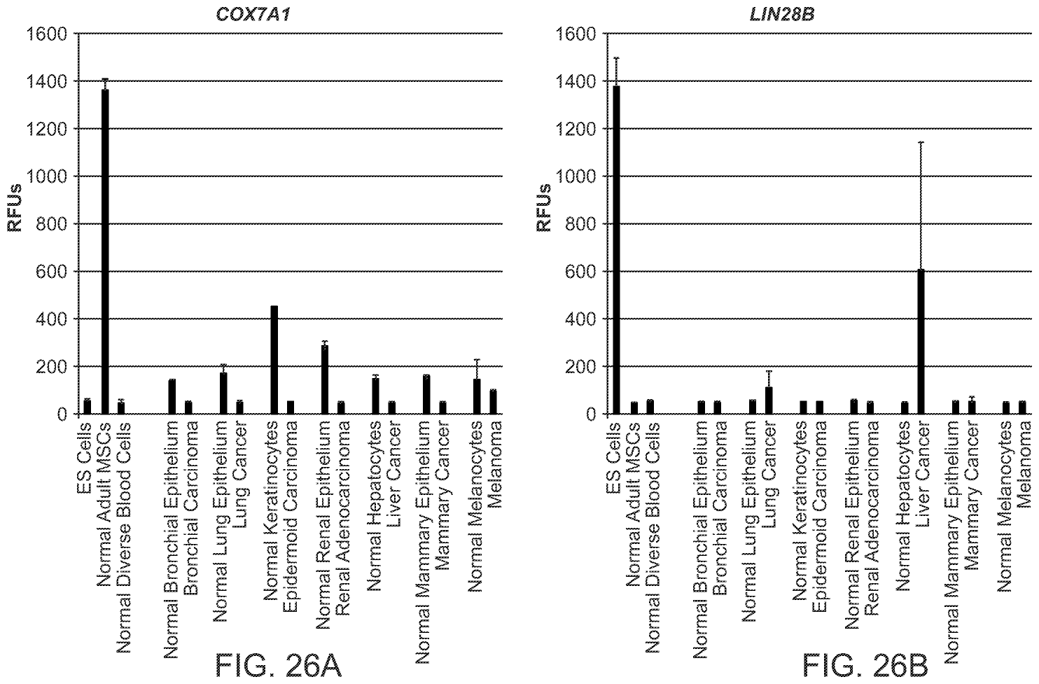

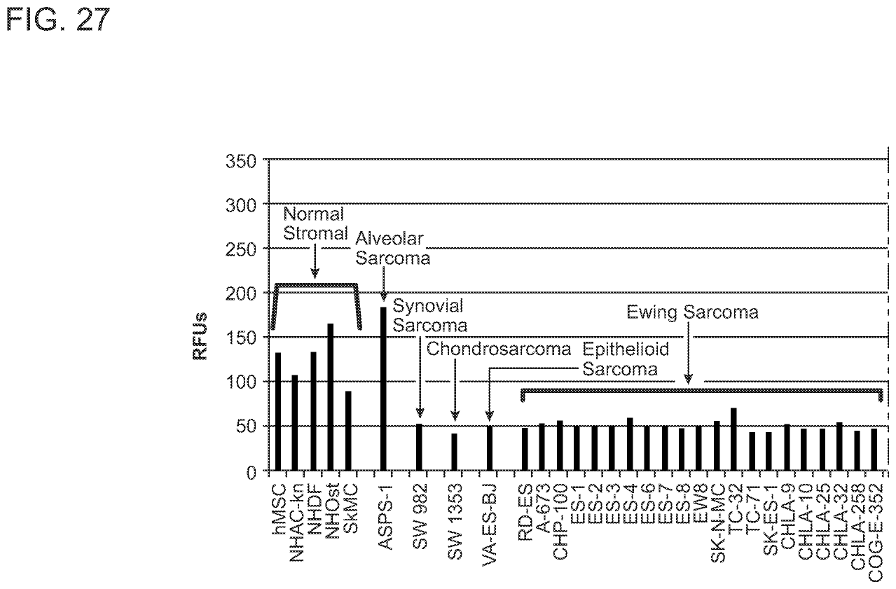

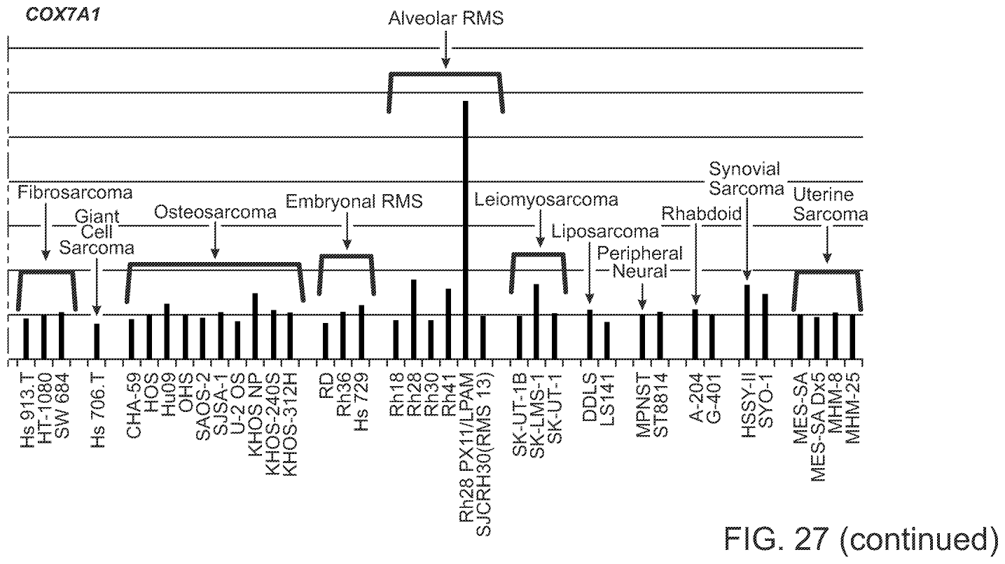

As shown in FIG. 24, LIN28A from two vendors, Genecopia (G) and other (O) were used with the Genecopoeia vector resulting in the highest expression levels of LIN28A (marked with “*”). Correlating with higher levels of LIN28A expression were lower levels of COX7A1, higher levels of GFER, and lower levels of CAT indicating a shift toward iTR. There was no detectable pluripotency markers such as HELLS or DNMT3B in these conditions. Therefore, LIN28A alone, or LIN28A in combination with TERT, or OCT4, KLF4, LIN28A, or OCT4, KLF4, LIN28A, and TERT were capable of inducing iTR.
Example 8. The use of LIN28B in conferring a fetal liver phenotype to adult blood cell stem and progenitor cell types.

As shown in FIG. 24, the gene LIN28B is normally expressed in most tissues only in early stages of embryonic development (minimal expression in early fetal skin cells shown in FIG. 7d), is expressed at relatively high levels in fetal liver-derived CD34+hematopoietic stem cells and CD36+ erythroid progenitors compared to adult-derived bone marrow (BM) and peripheral blood (PB) blood cells of diverse types as assayed by Illumina gene expression bead array. The cDNA for LIN28B is expressed in mouse and human CD34+ candidate hematopoietic stem cells and the relative proliferation of the cells in vitro compared to mock infected cells and the relative engraftment of the LIN28B expressing cells compared to mock transfected cells is compared to assay the extent of the benefit provided the cells in regard to proliferation and engraftment when LIN28B is expressed.

Sarcoma lines screened for sensitivity to chemotherapeutic agents show that markers of an fetal or adult state, such as COX7A1 expression (lines ASPS-1 and Rh28 PX11/LPAM, are uniquely resistant (typically 1-2 orders of magnitude higher IC50) to apoptosis in response to such chemotherapeutic agents as Teniposide, Paclitaxel, Etoposide, Valrubicin, Mitomycin C, Floxuridine, Sulfate, Clofarabine, Vinorelbine, Tartrate, Daunorubicin HC1. Expression of the gene or detection of its encoded protein or the gene's methylation status therefore is useful in predicting the response to diverse tumor types to chemotherapeutic agents.
Example 9. Producing mouse models of iTR

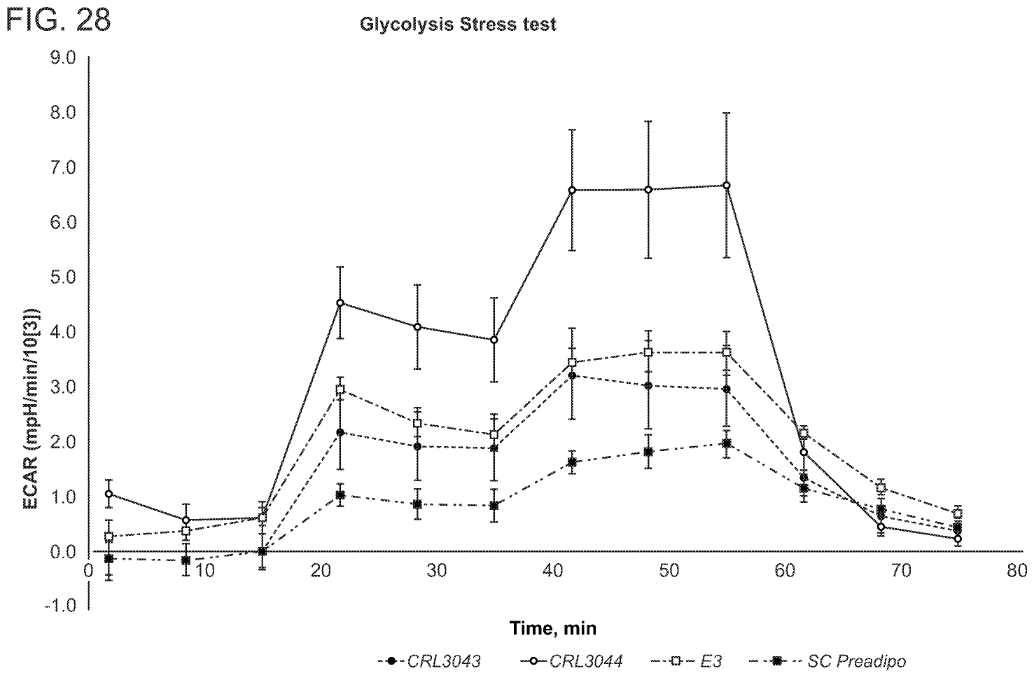

The Cox7a1 gene was knocked out in BL6 mice and the animals were bred to produce homozygous knockout animals (ko/ko). As shown in FIG. 28, human undifferentiated lipogenic cells from FIG. 8; namely, the clonal EP to white adipocytes designated E3, the adult preadipocytes to subcutaneous adipose tissue (SAT), and the liposarcoma cell lines CRL3043 and CRL 3044 were exposed to a glycolytic stress test in parallel. The highest extracellular acidification rate (ECAR) observed were in the cell types not expressing COX7A1 (i.e. E3, CRL 3043 and CRL 3044), and the lowest glycolytic shift was observed in the COX7A1 (adult-derived) SAT cells consistent with the highest Warburg shift occurring in the cells not expressing COX7A1. Next, the ko/ko mouse cells from the heart were compared to wt heart cells in the same glycolytic stress test measuring ECAR. As shown in FIG. 28, homozygous Cox7a1 ko/ko cells show a shift toward glycolysis with higher levels of extracellular acidification than wt cells indicative of a Warburg shift in metabolism. When the ko/ko mice were subjected to an ear puch assay, the ko/ko mouse ears showed accelerated wound healing compared to that of the wt mouse ears.

The mice with homozygous ko/ko of Cox7a1 are then bred with mice expressing other iTR genes including Lin28a to increase the robustness of TR in the animal model.
Example 10. Modulation of DNA methylation as a modality to impact iTR and iCM.

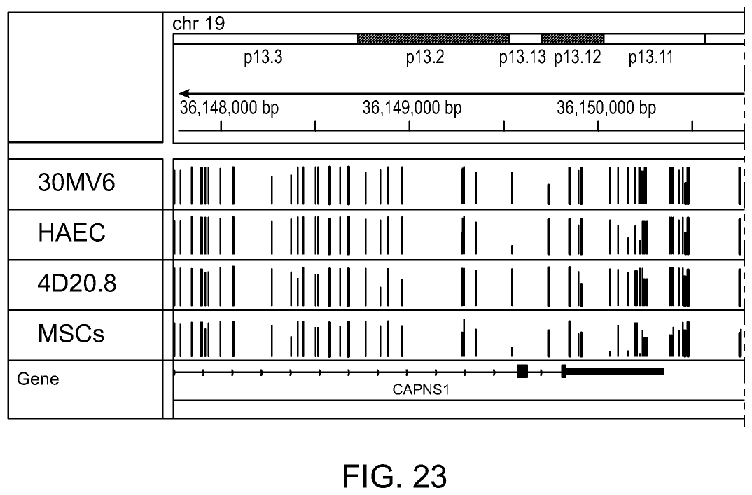

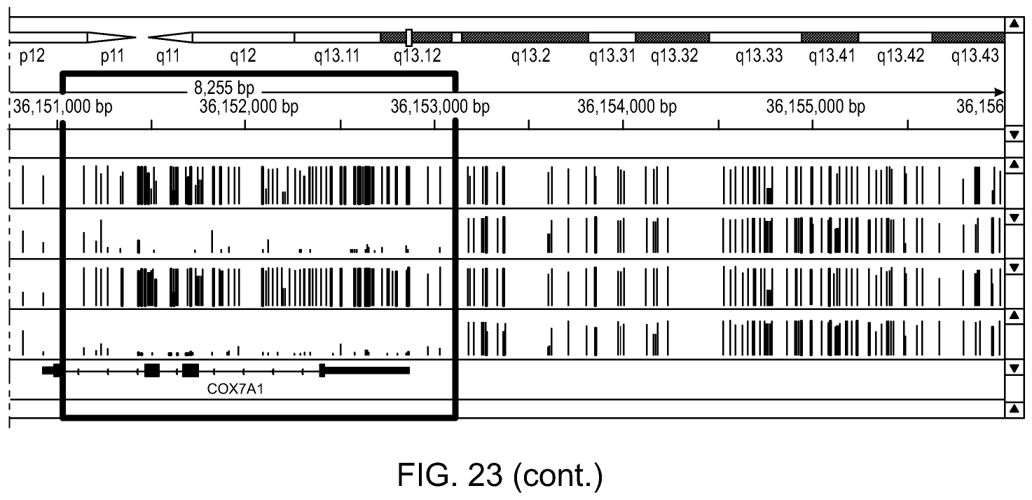

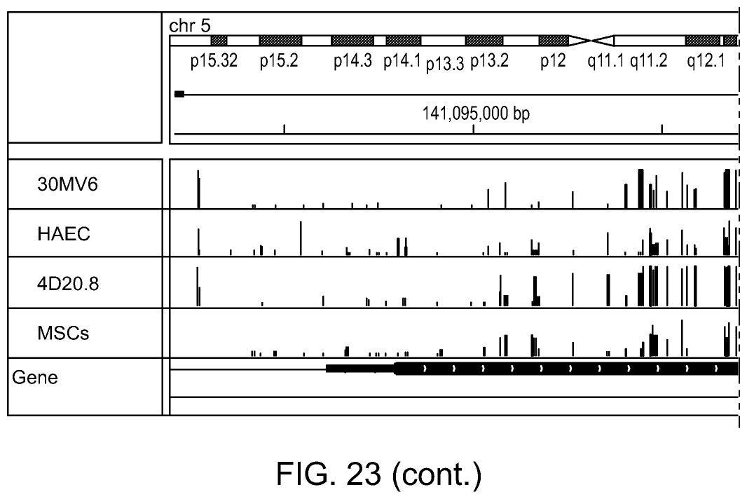

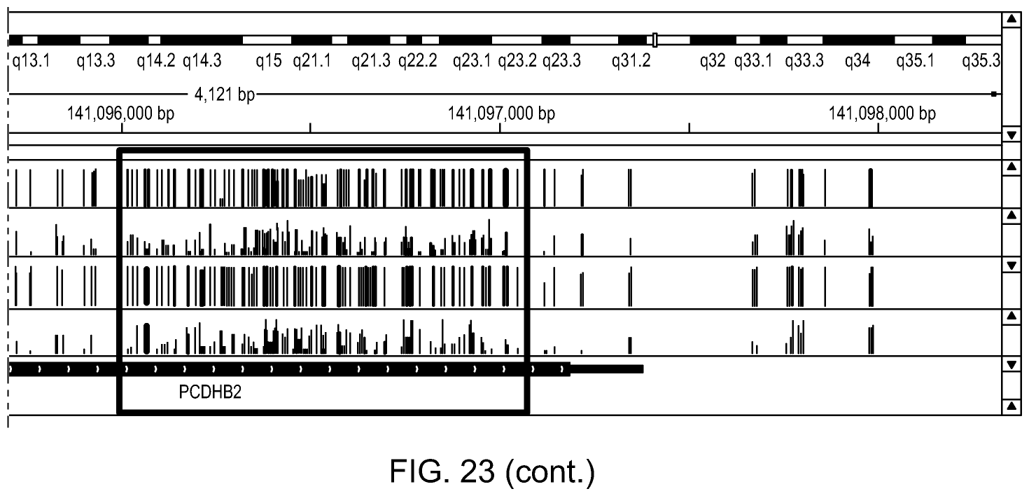

Global methylation patterns in genomic DNA from a human ES cell-derived clonal embryonic progenitor to vascular endothelium (30MV2) was compared to that of adult-derived human aortic endothelial cells (HAEC) and in parallel, global methylation patterns in genomic DNA from human ES cell-derived clonal embryonic progenitor to osteochondral cells (4D20.8) was compared to adult-derived one marrow mesenchymal stem cells (MSCs). As shown in FIG. 23, where the height of the bars in the histogram correspond to the percent of reads wherein the cytosine of CpGs were methylated, the COX7A1 gene is heavily methylated in both progenitor cell lines where COX7A1 expression could not be detected, and relatively demethylated in the corresponding adult-derived cell types where COX7A1 could be detected. Similar results were measured in the genes COMT, TRIM4, NAALADL1, TSPYL5, and PLPP7 providing novel evidence that inhibitors of DNA methyltransferase including but not limited to RG108; 5-aza-2′deoxycytidine (trade name Vidaza and Azadine), vitamin C (which can inhibit DNA methylation and increase Tet1 which increases 5 hmC which is a first step of demethylation) are useful global activators of iTR and that agents that increase methylation in these regions of the genome are effective at inducing iCM.
Example 11. Use of the GFER protein, AMH protein, and valproic acid to induce iTR.

The identification of the secreted protein iTR factors GFER and AMH as well as the agent valproic acid, provides a novel cocktail of factors to generate iTR in vitro as well as in vivo. To determine the effects of the factors, cultured adult-derived MDW fibroblasts as well as umbilical cord-derived MSCs (MSCwj) were treated with varying concentrations of the factors and the rate of growth and regrowth following a scratch test are performed.

The fibroblast line MDW (passage 5) is seeded in multiple wells of a 6 well plate and cultured to confluence. A 1.5 mm “scratch” was introduced onto the monolayer using a 200 ul pipette tip, thereby denuding the fibroblasts from the culture surface. Factors including 0.5 mM valproic acid, 10 ng/mL AMH, and 20 ng/mL GFER are added to the growth medium which as DMEM medium supplemented with 10% FBS. 24 hours later, the % of the denuded surface was determined by phase contrast microscopy. Control MDW cells in growth medium alone showed 20% coverage of the “wounded” zone. Cells treated with valproic acid alone showed improved regeneration of the wound with 25% regeneration, Cells treated with supplemented AMH alone showed 68% regeneration. Cells treated with GFER supplementation showed 28% regeneration. Cells treated with both valproic acid and AMH showed 50% regeneration. Cells treated with valproic acid, AMH, and GFER showed 50% regeneration. Due to the lability and diffusion of the factors when administered in vivo, the formulation of the factors alone or in combination in a hydrogel such as crosslinked hyaluronic acid or crosslinked hyaluronic acid and collagen provides a preferred method of delivering the factors to increase tissue regeneration in animals. Alternatively, umbilical cord-derived MSCs (MSCwj) and adult skin fibroblasts (MDW) each of which express fetal/adult markers such as COX7A1 are treated with the secreted form of GFER alone at concentrations of 0, 10.0 and 100 ng/mL in DMEM medium supplemented with 10% FBS an AMH

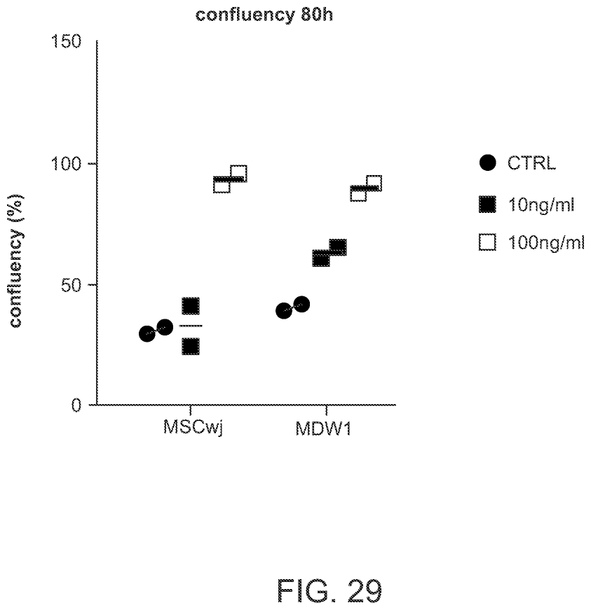

d cultured for 80 hours and the % confluency is determined. As shown in FIG. 29, there is a dose-dependent increase in confluency achieved in the presence of escalating doses of GFER, providing evidence of its utility in generating iTR in fetal and adult-derived stromal cells.

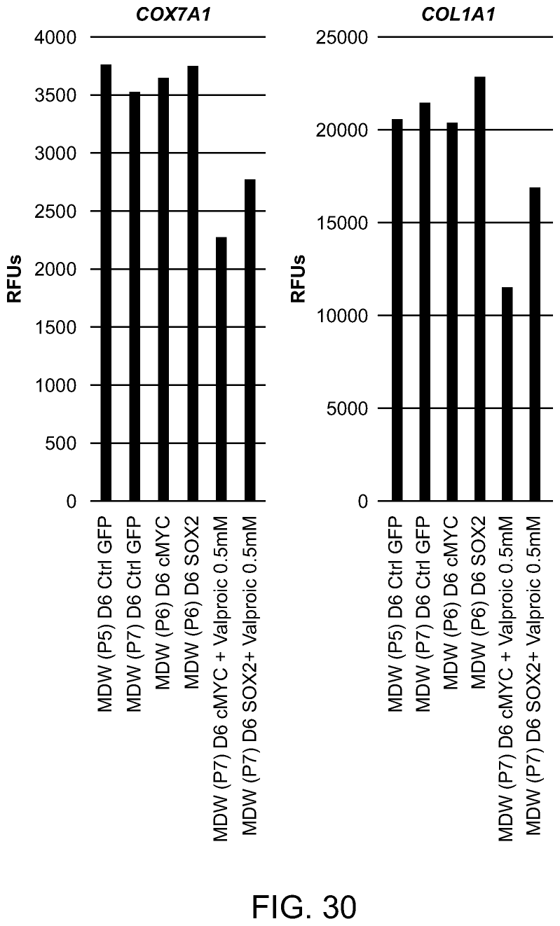

Adult skin fibroblasts (MDW (passage 6-7) were plated and incubated for 6 days in the presence of mRNA for GFP, cMYC, SOX2, or cMYC+0.5 mM vaproic acid, or SOX2+0.5 mM vaproic acid. As shown in FIG. 30, the addition of 0.5 mM valproic acid showed evidence of iTR as determined by the reduced expression of COX7A1, and decreased expression of the fibrosis marker COL1A1.

Induction of iTR can be achieved through a relative shifting of fetal or adult cells from a state of oxidative phosphorylation to anaerobic glycolysis through modification of the components of the MIA pathway, also known as the disulfide relay system of the mitochondrial intermembranous space. More specifically, this shift toward iTR can be induced in fetal or adult somatic cell types by increasing GFER protein levels in cells or decreasing COX7A1 protein levels in cells, or more preferably, by increasing GFER levels in cells and also decreasing COX7A1 levels. This modification can be accomplished by the expression of LIN28A, or by the exogenous administration of GFER or agents that increase levels of GFER in cells, along with agents that decrease COX7A1 protein levels. Even more preferably in humans is the induction of iTR in cells with increased levels of telomerase expression such as by the administration of the TERT gene, which can be in combination with, for example the induced expression of LIN28A.

The present disclosure also describes a means of intervention in mammalian aging whereby iTR is utilized to restore function in tissues afflicted with age-related degenerative disease. Preferably, the iTR is performed in combination with a lengthening of telomeres such as through the re-expression of telomerase activity.
Example 12. Use of fetal or adult cell-derived exosomes to generated iTM and iCM.

Exosomes from adult cells were tested for their ability to cause iTM using a hES cell-derived clonal embryonic vascular endothelial cell line 30-MV2-6. (See for example International Patent Application No. PCT/2012/054525, published as WO 2013/036969, and U.S. patent application Ser. No. 14/238,160, published as US 2014-0349396, all of which are incorporated herein by reference in their entirety.) Total RNA expression profile using 11lumina microarray analysis of a series of 15 hESC derived clonal embryonic progenitor cell lines and compared these to 18 primary endothelial cell lines (newborn to adult) obtained from various anatomical sites (not shown). Differentially expressed genes were retested using qPCR to assess the fold difference in RNA levels between an embryonic progenitor cell line, 30-MV2-6, and an umbilical cord derived cell line, HUVEC. COX7A1 showed the highest fold difference in gene expression between the two cell lines having markedly higher levels in HUVECs compared to the embryonic endothelial cell line 30-MV2-6. CAT and TRIM4 for were induced to a lesser extent. We incubated the 30-MV2-6 cell line in exosome-depleted medium to which HUVEC derived exosomes were added at a concentration of 1×10⁹ particles/ml for 24 h. The negative control 30-MV2-6 cells were incubated in exosome-depleted medium to which an equivalent volume of PBS was added. The HUVEC exosome treated 30-MV2-6 cells showed detectable expression of COX7A1 compared compared to undetectable expression in the PBS control. The embryonic genes, ACPS and LIN28B, appeared to be down-regulated after treatment of the embryonic progenitor cells with HUVEC exosomes. The results are indicative of the ability of exosomes from one developmental state to reprogram target cells to a different developmental state. In this case, treatment with adult cell exosomes results in a gene expression pattern that is similar to the adult pattern in the recipient embryonic cells. Significantly, COX7A1 is the most tightly regulated gene that we identified. It represents a marker of the embryonic to fetal transition being present in fetal to adult cells but absent in a wide variety of embryonic cell lines. The use of exosomes from fetal or adult-derived somatic cell types to iTM and iCM has practical application in introducing the EFT in vitro as well as in vivo and to mature cancer cell types, thereby increasing the availability of p53 to traffic to the nucleus and induce the expression of genes such as p21.

From the description herein, it will be appreciated that that the present disclosure encompasses multiple embodiments which include, but are not limited to, the following:

A method for regenerating damaged or aging tissue in a subject by contacting one or more cells of the subject with one or more induced tissue regeneration (iTR) factors.

The method of any previous embodiment, wherein the one or more iTR factors comprises a nucleic acid.

The method of any previous embodiment, wherein the nucleic acid comprises RNA.

The method of any previous embodiment, wherein the one or more iTR factors comprises an anti-Mullerian hormone (AMH).

The method of any previous embodiment, wherein one or more cells of the subject are contacted with the anti-Mullerian hormone (AMH) at a concentration of between 0.05 mM and 5 mM.

The method of any previous embodiment, wherein the one or more iTR factors comprises a protein encoded by the GFER gene.

The method of any previous embodiment, wherein one or more cells of the subject are contacted with the protein encoded by the GFER gene at a concentration of between 2 ng/mL and 200 ng/mL.

The method of any previous embodiment, wherein the one or more iTR factors comprises valproic acid.

The method of any previous embodiment, wherein one or more cells of the subject are contacted with valproic acid at a concentration of between 0.05 mM and 5 mM.

The method of any previous embodiment, wherein the one or more iTR factors are combined with a hydrogel.

The method of any previous embodiment, wherein the iTR factors increase GFER protein levels and decrease COX7A1 protein levels.

The method of any previous embodiment, wherein the iTR factors increase expression of LIN28A.

The method of any previous embodiment, further comprising increasing the expression of telomerase in the one or more cells of the subject.

The method of any previous embodiment, wherein administration of the TERT gene to the one or more cells of the subject increases expression of telomerase.

The method of any previous embodiment, wherein the iTR factors increase expression of LIN28A and increase expression of telomerase.

The method of any previous embodiment, wherein the subject is a human.

A method for repairing damaged or aging tissue in a subject by inducing an embryonic pattern of gene expression in one or more cells of the subject.

A kit for regenerating damaged or aging tissue in a subject, the kit comprising one or more of AMH, GFER protein and valproic acid iTF factors.

The kit of any previous embodiment, wherein the iTR factors are combined with a hydrogel.

A method for regenerating tissue in a subject by contacting one or more cells of the subject with an agent capable of inducing pluripotency, wherein pluripotency itself is not induced.

1.-20. (canceled)

21. A method for treating diseases or disorders of the eye in a subject in need thereof, the method comprising contacting one or more cells of the eye of the subject in vivo with one or more induced tissue regeneration (iTR) factors that comprise one or more nucleic acids encoding OCT4, SOX2, KLF4, NANOG, ESRRB, NR5A2, CEBPA, MYC, TERT, LIN28A, LIN28B, or any combination thereof, wherein the one or more cells of said damaged tissue do not revert to pluripotent stem cells, and wherein contacting the cells with one or more iTR factors is for 4 or 7 days.

22. A method for treating diseases or disorders of the eye in a subject in need thereof, the method comprising contacting one or more cells of the eye of the subject with one or more induced tissue regeneration (iTR) factors that comprise one or more nucleic acids encoding OCT4, SOX2, KFL4, TERT, or a combination thereof, wherein the one or more cells of said damaged tissue do not revert to pluripotent stem cells, and wherein contacting the cells with one or more iTR factors is for 4 or 7 days.

23. The method of claim 21, wherein the one or more iTR factors comprise nucleic acids encoding OCT4, SOX2, and KFL4.

24. The method of claim 21, wherein the one or more iTR factors comprise nucleic acids encoding OCT4, SOX2, KFL4, and TERT.

25. The method of claim 21, wherein the one or more iTR factors comprise RNA.

26. The method of claim 25, wherein the RNA is mRNA.

27. The method of claim 21, wherein the one or more iTR factors increase GFER protein levels and decrease COX7A1 protein levels in the one or more cells of the subject when compared to a control.

28. The method of claim 21, wherein the one or more iTR factors decrease expression of PLPP7 in the one or more cells of the subject when compared to a control.

29. The method of claim 21, wherein following administration of the one or more iTR factors to the subject, at least one regenerated cell does not express at least one pluripotent stem cell marker.

30. The method of claim 28, wherein the pluripotent stem cell marker is selected from HELLS and DNMT3B.

31. The method of claim 21, wherein contacting the cells with one or more iTR factors is for 4 days.

32. The method of claim 21, wherein contacting the cells with one or more iTR factors is 7 days.

33. The method of claim 21, wherein the subject is a human or a mouse.

34. The method of claim 21, wherein the disease or disorder of the eye is age-related macular degeneration, vision loss following eye injury, retinitis pigmentosa, or a neural retinal degeneration disorder.

35. The method of claim 21, wherein the one or more iTR factors are encoded by a viral vector.

36. The method of claim 35, wherein the viral vector is an adeno-associated virus vector.

37. The method of claim 21, wherein the one or more iTR factors are formulated for controlled release.

38. The method of claim 21, wherein the one or more iTR factors are combined with hydrogel.

39. The method of claim 21, wherein the one or more iTR factors are combined with cross-linked hyaluronic acid.

40. The method of claim 21, wherein the one or more iTR factors are administered topically, ocularly, or intra-ocularly.
